SEQUENCE LISTING (171 sequences)

SEQ ID NO: 1 - protein, 12 aa, artificial
  non-repeat portion of RAN-tranlsated polypeptide
LPHTAYLLLKNL

SEQ ID NO: 2 - protein, 7 aa, artificial
  non-repeat portion of RAN-tranlsated polypeptide
VKPGFLT

SEQ ID NO: 3 - protein, 16 aa, artificial
  non-repeat portion of RAN-tranlsated polypeptide
RVNLSVEAGSQKRQSE

SEQ ID NO: 4 - protein, 13 aa, artificial
  non-repeat portion of RAN-tranlsated polypeptide
ATRTLRAPFAGRG

SEQ ID NO: 5 - protein, 15 aa, artificial
  non-repeat portion of RAN-tranlsated polypeptide
SPAARGRARITGLEL

SEQ ID NO: 6 - protein, 18 aa, artificial
  non-repeat portion of RAN-tranlsated polypeptide
AVPRALSLPTGPRSRRQF

SEQ ID NO: 7 - protein, 12 aa, artificial
  non-repeat portion of RAN-tranlsated polypeptide
ITDHFFLSARLR

SEQ ID NO: 8 - protein, 11 aa, artificial
  non-repeat portion of RAN-tranlsated polypeptide
GSQTISFFRPG

SEQ ID NO: 9 - protein, 13 aa, artificial
  non-repeat portion of RAN-tranlsated polypeptide
GKLQAWEGSKPGR

SEQ ID NO: 10 - protein, 13 aa, artificial
  non-repeat portion of RAN-tranlsated polypeptide
LKGEFQHTGGRSL

SEQ ID NO: 11 - protein, 13 aa, artificial
  non-repeat portion of RAN-tranlsated polypeptide
SDLIKRQDEDRFA

SEQ ID NO: 12 - protein, 16 aa, artificial
  non-repeat portion of RAN-tranlsated polypeptide
LPACLPACLPACLPAC

SEQ ID NO: 13 - protein, 16 aa, artificial
  non-repeat portion of RAN-tranlsated polypeptide
QAGRQAGRQAGRQAGR

SEQ ID NO: 14 - protein, 39 aa, artificial
  RAN-tranlsated polypeptide
DNIFLKNAAAAAAAAAAAAAAAVVVVVVVVVVVKPGFLT

SEQ ID NO: 15 - protein, 15 aa, artificial
  RAN-tranlsated polypeptide
QQQQQQQQQQQQQQQ

SEQ ID NO: 16 - protein, 74 aa, artificial
  RAN-tranlsated polypeptide
YIFKKCSSSSSSSSSSSSSSSSSSSSSSSSSKARFSNMKDPGSSQGIGNRASANRVNLSVEAGSQKRQSECKDK

SEQ ID NO: 17 - protein, 32 aa, artificial
  RAN-tranlsated polypeptide
KTWLYYYYYYYYYYYCCCCCCCCCCCCCCCIF

SEQ ID NO: 18 - protein, 57 aa, artificial
  RAN-tranlsated polypeptide
SPIPNSLARPWVLHVRKPGFTTTTTTTTTTTAAAAAAAAAAAAAAAFFKNILSYFTI

SEQ ID NO: 19 - protein, 65 aa, artificial
  RAN-tranlsated polypeptide
LENLALLLLLLLLLLLLLLLLLLLLLLLLLLHFLKIYYLILLFDVIIVIYFSTLPHTAYLLLKNL

SEQ ID NO: 20 - protein, 43 aa, artificial
  RAN-tranlsated polypeptide
RPGREGPGPRPANGARRVLVAGNAAAAAGGITDHFFLSARLRP

SEQ ID NO: 21 - protein, 17 aa, artificial
  RAN-tranlsated polypeptide
LLLLLGGSQTISFFRPG

SEQ ID NO: 22 - protein, 118 aa, artificial
  RAN-tranlsated polypeptide
VPGARHRSRAHRLPVHNRSERGSPPSSSPVIRARPLAAGEGGAGSAAGERGSKGPCSRECCCCCWGDHRPFLSFGQAEALTWMGKLQAWEGSKPGRPCSILHAPPPIVGSQSAKLSCA

SEQ ID NO: 23 - protein, 82 aa, artificial
  RAN-tranlsated polypeptide
VCDPPSSSSSIPGYKDPSSPVRRPRTRPLPPRPLGGGPGSQDWSWAETHARSGCELAGGGRGFCAVPRALSLPTGPRSRRQF

SEQ ID NO: 24 - protein, 116 aa, artificial
  RAN-tranlsated polypeptide
GGGRGIPEKAGLAKANFPSKQAEIAPDAPQSRASCTRKLCTLRTNDRWGCVEDGTRTARLAAFPGLQFAHPRQGLSLAERKKWSVIPPAAAAAFPATRTLRAPFAGRGPGPSLPGR

SEQ ID NO: 25 - protein, 51 aa, artificial
  RAN-tranlsated polypeptide
SPQQQQQHSRLQGPFEPRSPAADPAPPSPAARGRARITGLELGGDPRSERL

SEQ ID NO: 26 - protein, 90 aa, artificial
  RAN-tranlsated polypeptide
VLLPVCVCVCVCVCVCVCLSVCLSVCLSVCLPACLPACLPGCLSACLPACLPACLPVCLTLSPRLECSGMISAHCNLHPPGSSDSSASAS

SEQ ID NO: 27 - protein, 72 aa, artificial
  RAN-tranlsated polypeptide
VNEYYCQCVCVCVCVCVCVSVCLSVCLSVCLSACLPACLPACLAACLPACLPACLPACLSVSLCPLGWSAVV

SEQ ID NO: 28 - protein, 63 aa, artificial
  RAN-tranlsated polypeptide
SITASVCVCVCVCVCVCLSVCLSVCLSVCLPACLPACLPAWLPVCLPACLPACLPACLSHFVP

SEQ ID NO: 29 - protein, 81 aa, artificial
  RAN-tranlsated polypeptide
AEIIPLHSSLGDKVRQTGRQAGRQAGRQADRQPGRQAGRQAGRQTDRQTDRQTDRQTHTHTHTHTHTHTGSNTHSLIPSPT

SEQ ID NO: 30 - protein, 77 aa, artificial
  RAN-tranlsated polypeptide
DRQAGRQAGRQAGRQTGSQAGRQAGRQAGRQTDRQTDRQTDRHTHTHTHTHTHTLAVILIHSFQVQLNGHICMVIRP

SEQ ID NO: 31 - protein, 79 aa, artificial
  RAN-tranlsated polypeptide
TRGVEVAVSRDHTTALQPRGQSETDRQAGRQAGRQAGRQAARQAGRQAGRQADRQTDRQTDRQTDTHTHTHTHTHTHWQ

SEQ ID NO: 32 - protein, 131 aa, artificial
  RAN-tranlsated polypeptide
AAGTGPRWTAAQVLLLPAAQSPIHCPGAERRRESARGLRGLPCRAGDRHGDPGKADEGLRVPQVLPAAAAAAAAAAAAAAAAAAAAATAATAAAAAAASSASSAAAAGTAAAASAAAAPAAAPAATRPGCG

SEQ ID NO: 33 - protein, 93 aa, artificial
  RAN-tranlsated polypeptide
RPSSPSSPSSSSSSSSSSSSSSSSSSSSSSNSRHRRRRRRRLLSFLSRRRRHSRCCLSRSRPRRRPRRHPARLWLRSRCTDQRKNFQLPRKTV

SEQ ID NO: 34 - protein, 83 aa, artificial
  RAN-tranlsated polypeptide
GGGGGGGGGGGCCCCCCCCCCCCCCCCCCCCCCWKDLRDSKAFISFSRVAMAVSRPARQSPEASGRLAAPLSTGAMNGALGRR

SEQ ID NO: 35 - protein, 139 aa, artificial
  RAN-tranlsated polypeptide
GSGAEVGEGLAPGGGGCPSWALGCWVTLSLRGRGFVSPARRLQGYRHPRRSLGPAGTGSCSGPKLTVGAAAPQPQPGRVAAGAAAGAAAAEAAAAVPAAAAEEAEEAAAAAAAVAAVAAAAAAAAAAAAAAAAAAAGRT

SEQ ID NO: 36 - protein, 220 aa, artificial
  RAN-tranlsated polypeptide
SVQRLLSHSRAGWRRGRRRGRLRLRQQRLCLRRRLRKLRRRRRRRRRWRLLLLLLLLLLLLLLLLLLLLLLLEGLEGLEGLHQLFQGRHGGLPPGTAVPGGLGPTRGAAQHRGNEWGSGPQVKAEPERPSILDPSRQPPRRLASQTLRRRRRGRAGGGGATPASMIDSPSLRTLPMAGQGTSPPLPPQVLPHTARPLTAQRPTRAKARGSTERGRGVVRL

SEQ ID NO: 37 - protein, 103 aa, artificial
  RAN-tranlsated polypeptide
RVRCTEEWISESPGRRAAAEPAKVPCTETILQQQQQQQQQQQQQQQQAAAAAAAAAAAAAAAAAAAAAAAAAAAAAAAAAAAAAAAGSSLASGPGSAPNAVAS

SEQ ID NO: 38 - protein, 139 aa, artificial
  RAN-tranlsated polypeptide
APTWEWTGRGSQFVWGLASRKGPAPCVSGALRSGYRRVQAGGQLQSRPRFPAQKPSYSSSSSSSSSSSSSSSRQQQQQQQQQQQQQQQQQQQQQQQQQQQQQQQQQQQQQQQAAPWLPAPALPRMRWHLRMKAQIDSLN

SEQ ID NO: 39 - protein, 99 aa, artificial
  RAN-tranlsated polypeptide
GVDIGESRQAGSCRAGQGSLHRNHLTAAAAAAAAAAAAAAAGSSSSSSSSSSSSSSSSSSSSSSSSSSSSSSSSSSSSSSRQLPGFRPRLCPECGGILE

SEQ ID NO: 40 - protein, 82 aa, artificial
  RAN-tranlsated polypeptide
LRESICAFILRCHRIRGRAGAGSQGAACCCCCCCCCCCCCCCCCCCCCCCCCCCCCCCCCCCCCCCLLLLLLLLLLLLLLLL

SEQ ID NO: 41 - protein, 154 aa, artificial
  RAN-tranlsated polypeptide
DATAFGAEPGPEARELPAAAAAAAAAAAAAAAAAAAAAAAAAAAAAAAAAAAAAAACCCCCCCCCCCCCCCCKMVSVQGTLAGSAAARLPGLSDIHSSVHLTRMEPVLSWKPDPKQTGFPDQSTPMWELILRGTGSSASLGRLACFRHGCLGRE

SEQ ID NO: 42 - protein, 99 aa, artificial
  RAN-tranlsated polypeptide
PPHSGQSRGRKPGSCLLLLLLLLLLLLLLLLLLLLLLLLLLLLLLLLLLLLLLLPAAAAAAAAAAAAAAAVRWFLCREPWPALQLPACLDSPISTPQCT

SEQ ID NO: 43 - protein, 58 aa, artificial
  RAN-tranlsated polypeptide
SSSSSSSSSSCECARVGVRVSALAPAAAPCPAPRQLPYPRLPEPPSRGTSTLIPARQA

SEQ ID NO: 44 - protein, 26 aa, artificial
  RAN-tranlsated polypeptide
CTSRLQPPAAAAAAAAAAAASARVWV

SEQ ID NO: 45 - protein, 209 aa, artificial
  RAN-tranlsated polypeptide
TCKLVACCPGADSRLHAPHCSKEQPQPLPRPPLLLGKSRGAADAVGRSLAFNAPAASSLLQQQQQQQQQQLRVRACGCEGECAGAGCSALPSSPPASLPPPAGAALPWDQHPHSGQASLNPVPAISPLQLGSRKAPFCRGCPLSQGEEGSFLHFGAAAKEGNLLGISPDPLVSASGAGGRDQTPRGTAYLGTRRQRRQAQHPRWELELE

SEQ ID NO: 46 - protein, 84 aa, artificial
  RAN-tranlsated polypeptide
VPFWTRAAVARWQGQDSGLPGRNEGAGPTGGRLRQAGVGKLAGSWAGRCSRRQRTHPHTHTRALAAAAAAAAAAAAGGWRRLVH

SEQ ID NO: 47 - protein, 93 aa, artificial
  RAN-tranlsated polypeptide
GSWRGAGQGAAAGASALTLTPTRAHSQLLLLLLLLLLQEAGGGWCIKGEAPPNRVSSPTTLSQQQWWPRQRLRLLFAAVGRMQPRIGTWAASD

SEQ ID NO: 48 - protein, 142 aa, artificial
  RAN-tranlsated polypeptide
GCWSHGRAAPAGGGREAGGELGRALQPAPAHSPSHPHARTRSCCCCCCCCCCRRLEAAGALKARLLPTASAAPRLFPSSSGGRGRGCGCSLLQWGACSRESAPGQQATSLQVQVERIDALFQPPPLSRHSAKGHVYASTQSP

SEQ ID NO: 49 - protein, 63 aa, artificial
  RAN-tranlsated polypeptide
TASAGGGGDGGAAARGRAAARRRRRRRRRRRRRRRRRRRRLGLERPQPTSRGRAPGASRAEEK

SEQ ID NO: 50 - protein, 82 aa, artificial
  RAN-tranlsated polypeptide
RRARAAAVTEAPLPGGVRQRGGGGGGGGGGGGGGGGGGGGWASSARSPPLGGGLPALAGLKRRWRSWWWKCGAPMALSTRHL

SEQ ID NO: 51 - protein, 48 aa, artificial
  RAN-tranlsated polypeptide
RRRRCQGACGSAAAAAAAAAAEAAAAAAAAAAGPRAPAAHLSGAGSRR

SEQ ID NO: 52 - protein, 56 aa, artificial
  RAN-tranlsated polypeptide
RREPAPERWAAGARGPAAAAAAAAAASAAAAAAAAAALPHAPWQRRLRHRRRPRSP

SEQ ID NO: 53 - protein, 40 aa, artificial
  RAN-tranlsated polypeptide
PPPPPPPPPPPPPPPPPPPPRCRTPPGSGASVTAAARARR

SEQ ID NO: 54 - protein, 77 aa, artificial
  RAN-tranlsated polypeptide
KAPLEPRTSTTSSSIFSSALLAPGARPREVGCGRSRPSRRRRRRRRRLRRRRRRRRRRAAARPLAAAPPSPPPPALA

SEQ ID NO: 55 - protein, 90 aa, artificial
  RAN-tranlsated polypeptide
VEDSAKLKDGSAVRAGKGLPSAAVQDLPRSFPESVPERARSDPEPGPQAPRGRERSTSRRQFAAAAAAAAAAAAAAAAAAAAAAAAAARD

SEQ ID NO: 56 - protein, 140 aa, artificial
  RAN-tranlsated polypeptide
SRTRAPGTQRPRAQHLPAPVCCCCSSSSSSSSSSSSSSSSSSSSSSKRLAPGSSSSSRVRMVLPKPIVEAPQATWSWMRNSNLHSRSRPWSATPREVASQSLEPPWPPARGCRSSCQHLRTRMTQLPHPRCPCWAPLSPA

SEQ ID NO: 57 - protein, 168 aa, artificial
  RAN-tranlsated polypeptide
SLAAAAAAAAAAAAAAAAAAAAAAAAAANWRREVLRSRPLGAWGPGSGSLRARSGTDSGKLLGRSWTAAEGRPFPALTALPSLSLAESSTYFPYPASPSLAQKSSTGCDDAVVAAASCPPAGSSREGNLREQPRKKRSDSLKVSCRRRHTVDKICPARLTVCLLILEG

SEQ ID NO: 58 - protein, 94 aa, artificial
  RAN-tranlsated polypeptide
GLGRTILTLLLLLLPGASLLLLLLLLLLLLLLLLLLLLLLLLQQQQTGAGRCCARGLWVPGARVLDHFAHALEQILESSSVGLGRRPRVDPSQP

SEQ ID NO: 59 - protein, 87 aa, artificial
  RAN-tranlsated polypeptide
PVGPLRWAWGEPSSPCCCCCCLGLVSCCCCCCCCCCCCCCCCCCCCCCCSSSKLAPGGAALAASGCLGPGFWITSRTLWNRFWKAPR

SEQ ID NO: 60 - protein, 51 aa, artificial
  RAN-tranlsated polypeptide
SSSSSSSSSSSSSSSITETLGPLLLEHFPTHWRAVAPTTHTLTPCLPPWGL

SEQ ID NO: 61 - protein, 34 aa, artificial
  RAN-tranlsated polypeptide
AAAAAAAAAAAAAAASRKLWAPSSWSISPPTGGR

SEQ ID NO: 62 - protein, 21 aa, artificial
  RAN-tranlsated polypeptide
CCCCCCCCCCCCCCCCCCCWW

SEQ ID NO: 63 - protein, 37 aa, artificial
  RAN-tranlsated polypeptide
AAAAAAAAAAAAAAAVAVAGGDGDVLRLVGGRWTGPQ

SEQ ID NO: 64 - protein, 25 aa, artificial
  RAN-tranlsated polypeptide
LLLLLLLLLLLLLLLLLLLVVMVMC

SEQ ID NO: 65 - protein, 113 aa, artificial
  RAN-tranlsated polypeptide
AAAAAAAAAAAAASASAAAAAAAAAAAAAAPQQGSGAHHPGVPPTSPAEPVRPHFQFSAEHRPHRLSSGHPRPPPPPPDDDPTHAHPGAPLPGRHAIRRLRQPLCPSGGHQES

SEQ ID NO: 66 - protein, 88 aa, artificial
  RAN-tranlsated polypeptide
ARRRDTRLSSSSSSSSSSSSSISISSSSSSSSSSSSSSTSAGLRGSSPRGPPHQPSRTSTSTFPVLRRTPAAPPLLRPSPSTSTPTRR

SEQ ID NO: 67 - protein, 32 aa, artificial
  RAN-tranlsated polypeptide
CCCCCCCCCCCCCSALCPGVWLRLPMLASRVE

SEQ ID NO: 68 - protein, 71 aa, artificial
  RAN-tranlsated polypeptide
GAAAAAAAAAAAAAADADAAAAAAAAAAAAAQPCVPASGSDCPCWPAEWNRPPAGSAGMEWWPLRPRPLHW

SEQ ID NO: 69 - protein, 193 aa, artificial
  RAN-tranlsated polypeptide
GRGPVPPALLHLTVQDLLGLDGLLQPAALSFLGGLPRDKVAAGVGVLHDDLGGGPQGERVWDHRLVGVEVDGDGRRRGGAAGVLRRTGNVDVLVLLGWWGGPRGDEPRSPAEVLLLLLLLLLLLLLLMLMLLLLLLLLLLLLLSLVSRRLAQTAHVGQQSGIGLQLGALGWSGGPCGRGHCTGDGVGGWGDQL

SEQ ID NO: 70 - protein, 41 aa, artificial
  RAN-tranlsated polypeptide
SPSSSSSSSSSSSSSNSSSSSSSSSRRPRLPMSASPAAAAF

SEQ ID NO: 71 - protein, 169 aa, artificial
  RAN-tranlsated polypeptide
QRQRRRRVSARLPAAPWSRRASPPLRRPPSPPRQPGRPSGRANPRLPARRPRVPAAFRRLLGAPGSRLSPPGVRAGVWAPHHVAEAPAAAAAAAAAAAAATAAAAAAAAAAARGCQCPQARRQRPSSVARRRAFAVLVLGLLVLGHGSLLGGRGDLRRREARPGQRSKQ

SEQ ID NO: 72 - protein, 130 aa, artificial
  RAN-tranlsated polypeptide
KAAAAGLADIGSRGRRLLLLLLLLLLLLLLLLLLLLLLLGLQRHGEGPIHRLARRAGTAGSRARQGDAGTRRGRAGAERGGAGWRGRRGARAGEGEKEDDEGAGRPAETKEPPGAGPKRAAAVAVATKTV

SEQ ID NO: 73 - protein, 268 aa, artificial
  RAN-tranlsated polypeptide
GSPLLLFRPLPRPGLPPPEVAATTEEGAVAEDEETEDEDGEGAAAGDARRPLPPGLRTLAAAGGGCCCCCCCCCCCCCCCCCCCCCCWGFSDMVRGPYTGSHAGRGQPGAGRAKETPERGGDARAPSGEARVGAAGGAPGLARGRRRTTKGRGGPPRPRSRREPGRNAPPPLPLLPKQSEAEGGELCREGGGPGPGGGGAAEGYGPGAAPPPPRPLRRAGRWSERHPGHLAAAKRRDSVATAGLRGAAAAERIGGRARRGAGWERRCG

SEQ ID NO: 74 - protein, 95 aa, artificial
  RAN-tranlsated polypeptide
TEAVLCYCFDLCPGRASRRRRSPRPPRREPWPRTRRPRTRTAKARRRATLEGRCRRACGHWQPRAAAAAAAAAAAVAAAAAAAAAAAAAGASATW

SEQ ID NO: 75 - protein, 61 aa, artificial
  RAN-tranlsated polypeptide
HRHQVQILLQKSFGRDEKPTLKNSSKSSNSSSSSSSRGTYQDRVHIHVKGQPPVQEHLGVI

SEQ ID NO: 76 - protein, 26 aa, artificial
  RAN-tranlsated polypeptide
KTAAKAATAAAAAAAGGPIRTEFTSM

SEQ ID NO: 77 - protein, 29 aa, artificial
  RAN-tranlsated polypeptide
VPLLLLLLLLLLLLLLFFKVGFSSLPKLF

SEQ ID NO: 78 - protein, 17 aa, artificial
  RAN-tranlsated polypeptide
CCCCCCCCCCFCCCFSK

SEQ ID NO: 79 - protein, 28 aa, artificial
  RAN-tranlsated polypeptide
AAAAAAAVAAFAAVFQSRLLVSSEALLK

SEQ ID NO: 80 - protein, 304 aa, artificial
  RAN-tranlsated polypeptide
SVRPAARGPRSSSSSSSSSSSSRRWPGRAGRPPAALGGTQAPRPSLWPEIGRPRGATAAAARPGWRGGSQARPGASPPGPVDTAGPGGRHLARTCPRGPRVPGTMATTGAPTTTRPMARAAGAARRPWPGPTTRHPPYDTRPRAPPGARPGLPGPRARPAPRLLGTAGDSPTATTRRTDWPGPAGRAPGRACTNPTARVTMIGAKPGRGGARPAPHAPHAHTPPEEPRRGRGGPAQRARERASRETPDSGEARAGPQGCPAETLGQKRPSWAATAPPNQPRSPHPRQGLSGGRQGADKPHSQGI

SEQ ID NO: 81 - protein, 196 aa, artificial
  RAN-tranlsated polypeptide
GRRLGAPAAAAAAAAAAAAAGGGQAGPGGHQRPSEVPRPHGRASGRRSAAHGGPQQRPLAQDGEAGPRPGPERVPQGLSTRRGPVAGIWPARVRGAPGSPAPWLLPGLRLRRGRWPGQRGRRGGHGRGLRRATPRTTRVLGRHRALAQDSPGLGPGLRLAFSARPATPQRLLPGARTGQAPRAGLQEGPARTLQRE

SEQ ID NO: 82 - protein, 214 aa, artificial
  RAN-tranlsated polypeptide
PPAAAAAAAAAAAAAGAPSRRPYGSQGNRTRVAGGWRGSGGAGGGPAAECWYQMLRGLGFHLRNYCPAGAPCALGPRWASGTSAGPEPVPGRGPAVPGHSGPCRGAGDGGGGGGGGGAVDASDPWAGPAPGPAPSTAGPRIGWSCSGLSPSLCPHRCSGPGSAQRRGGCGRGAAGGAAGSPRAGPAPASNRPGVGPWGPARRLNASWGWCLRWY

SEQ ID NO: 83 - protein, 53 aa, artificial
  RAN-tranlsated polypeptide
LLLLLLLLLLLLLRGPRAAGLTDHRGIGHVWPGGGGGLGELAAAPPRSAGTRC

SEQ ID NO: 84 - protein, 26 aa, artificial
  RAN-tranlsated polypeptide
CCCCCCCCCCCCCGGPEPPALRITGE

SEQ ID NO: 85 - protein, 58 aa, artificial
  RAN-tranlsated polypeptide
AAAAAAAAAAAAASAAPAAAAPATAATAHTAGGRRARRRLHLGRRNGDGRGAQASAQS

SEQ ID NO: 86 - protein, 59 aa, artificial
  RAN-tranlsated polypeptide
SSSSSSSSSSRRLRSPSGSSTRHRRHGAHGRRTAGPAPPPPRPPQWRRSGSAGLCPVLK

SEQ ID NO: 87 - protein, 68 aa, artificial
  RAN-tranlsated polypeptide
GGGGGCCCRWGCGGGGCCCCCCCCCCRAAAAAAPPAAAAARRGSPLTSSAARSDILSAPFLWRVGQKS

SEQ ID NO: 88 - protein, 106 aa, artificial
  RAN-tranlsated polypeptide
NFRPILSRPSQEVWKPQPTDSTTVPASLQDWAEACAPRPSPLRRPRWRRRRARRPPAVCAVAAVAGAAAAGAAEAAAAAAAAAAAAAGRPRPLLRPPPPPRGAAPP

SEQ ID NO: 89 - protein, 65 aa, artificial
  RAN-tranlsated polypeptide
LLLLLLLLLLPGGRGRCSARRRRRAARLPPDVIRGPLRHSFRSFSLEGRPKILINLPMDLHLLQL

SEQ ID NO: 90 - protein, 104 aa, artificial
  RAN-tranlsated polypeptide
PHSLFRTPIVCLFWKSNKGSSSNNNSSSSSSSSNSNSSSSSSSSSSSSSSSSSSSNRQWQLQPFSSQRPSRQHREPQARHHSSSTHRLSQLHPCRAPLHCIPPP

SEQ ID NO: 91 - protein, 128 aa, artificial
  RAN-tranlsated polypeptide
SVYFGRATKAAAATTTAAAAAAAATATAAAAAAAAAAAAAAAAAAATGSGSCSRSAVNVPAGNTGNLRPGTTALPLTDSHNCTLAGHHSTVSLPHDSHDPHHSCHASFGEFWDCTAAAKYCIHSESWL

SEQ ID NO: 92 - protein, 197 aa, artificial
  RAN-tranlsated polypeptide
LLNGCSCHCLLLLLLLLLLLLLLLLLLLLLLLLLLLLLLLLLLLLLLPLLLFQNRQTIGVLNRLWGQSSAIRHHWTKDRDSGSHGTLRGGQALSVRWQAVVLIHDVHFLLGKPETLALELVSLFNFFLEHLQHTLLSNFLNSLGYLHTPRNSDAGSLQRSLWASGSEVKQPAAQAPATANLPDLTEPLARVDNVTSA

SEQ ID NO: 93 - protein, 58 aa, artificial
  RAN-tranlsated polypeptide
TAAAATACCCCCCCCCCCCCCCCCCCCCCCCCCCCCCCCCCCCCCLCCSSKIDRLLVF

SEQ ID NO: 94 - protein, 76 aa, artificial
  RAN-tranlsated polypeptide
ESVSGRAVVPGLRFPVLPAGTLTAERLQLPLPVAAAAAAAAAAAAAAAAAAAVAVAAAAAAAAVVVAAAAFVALPK

SEQ ID NO: 95 - protein, 115 aa, artificial
  RAN-tranlsated polypeptide
NPNRLPSGALSCCCCCCCCCCCCCCCCCCCCCCCCCSSSSSSFSSSSSSSRPSFGEMAFGSFARKRSPRQAALQPPFCLLHFLHSFLCFLQALTQGRCALSTRYVEEEGNQLGSK

SEQ ID NO: 96 - protein, 101 aa, artificial
  RAN-tranlsated polypeptide
KESTKHTNKIQTAFQVGLFHAAAAAAAAAAAAAAAAAAAAAAAAAPPPPPPSPPPPPLLDLLLEKWLSEVLPGNVALGRQLCSPLSACCTFSIRSFAFCRL

SEQ ID NO: 97 - protein, 73 aa, artificial
  RAN-tranlsated polypeptide
KRRRRRRRRRRRRSSSSSSSSSSSSSSSSSSSSSSSSSMKEPHLEGGLDFICVFCGFFLFCFTNASYTKLIWH

SEQ ID NO: 98 - DNA, 35 bp, artificial
  oligonucleotide primer
cgaaccaagcttatcccaattccttggctagaccc

SEQ ID NO: 99 - DNA, 36 bp, artificial
  oligonucleotide primer
acctgctctagataaattcttaagtaagagataagc

SEQ ID NO: 100 - DNA, 10 bp, artificial
  sequence between the stop codon cassette and the CAG repeat tract
tagaattcag

SEQ ID NO: 101 - DNA, 42 bp, artificial
  oligonucleotide primer
agttaagctagcttagctaggtaactaagtaactagaattaa

SEQ ID NO: 102 - DNA, 33 bp, artificial
  oligonucleotide primer
tagaaggcacagtcgaggctgatcagcgggttt

SEQ ID NO: 103 - DNA, 45 bp, artificial
  oligonucleotide primer
agttaagctagcttagctaggtaactaagtaactagaatagagca

SEQ ID NO: 104 - DNA, 33 bp, artificial; identical to SEQ ID NO: 102
  oligonucleotide primer

SEQ ID NO: 105 - DNA, 53 bp, artificial
  oligonucleotide primer
gaattatgggtaagcctatccctaaccctctcctcggtctcgattctacggga

SEQ ID NO: 106 - DNA, 51 bp, artificial
  oligonucleotide primer
aattcccgtagaatcgagaccgaggagagggttagggataggcttacccat

SEQ ID NO: 107 - DNA, 25 bp, artificial
  oligonucleotide primer
ctcgaggctacaaggacccttcgag

SEQ ID NO: 108 - DNA, 26 bp, artificial
  oligonucleotide primer
cctgaaccctagaactgtcttcgact

SEQ ID NO: 109 - DNA, 33 bp, artificial; identical to SEQ ID NO: 102
  oligonucleotide primer

SEQ ID NO: 110 - DNA, 41 bp, artificial
  oligonucleotide primer
agttaagcttagctaggtaactaagtaactagaactcagca

SEQ ID NO: 111 - DNA, 56 bp, artificial
  oligonucleotide primer
agttaagcttagctaggtaactaagtaactagaatttcctgcacagaaaccacctt

SEQ ID NO: 112 - DNA, 40 bp, artificial
  oligonucleotide primer
agttaagcttagctaggtaactaagtaactagaacttcct

SEQ ID NO: 113 - DNA, 56 bp, artificial
  oligonucleotide primer
agttaagcttagctaggtaactaagtaactagaacttcctgcacagaaaccacctt

SEQ ID NO: 114 - DNA, 40 bp, artificial
  oligonucleotide primer
agttaagcttagctaggtaactaagtaactagaactaaca

SEQ ID NO: 115 - DNA, 40 bp, artificial
  oligonucleotide primer
agttaagcttagctaggtaactaagtaactagaacttcga

SEQ ID NO: 116 - DNA, 42 bp, artificial
  oligonucleotide probe
tagaaggcacagtcgaggctgatcagcgggtttaaactcaat

SEQ ID NO: 117 - DNA, 30 bp, artificial
  oligonucleotide primer
cagatcctcttctgagatgagtttttgttc

SEQ ID NO: 118 - DNA, 21 bp, artificial
  oligonucleotide primer
acccaagctggctagttaagc

SEQ ID NO: 119 - DNA, 20 bp, artificial
  oligonucleotide primer
tgtcgtcgtcgtccttgtaa

SEQ ID NO: 120 - DNA, 20 bp, artificial
  oligonucleotide primer
tcgtgcgtgacattaaggag

SEQ ID NO: 121 - DNA, 20 bp, artificial
  oligonucleotide primer
gatcttcattgtgctgggtg

SEQ ID NO: 122 - DNA, 23 bp, artificial
  oligonucleotide primer
cgactggagcacgaggacactga

SEQ ID NO: 123 - DNA, 30 bp, artificial
  oligonucleotide primer
cgcctgccagttcacaaccgctccgagcgt

SEQ ID NO: 124 - DNA, 31 bp, artificial
  oligonucleotide primer
gaccatttctttctttcggccaggctgaggc

SEQ ID NO: 125 - DNA, 28 bp, artificial
  oligonucleotide primer
gcagcattcccggctacaaggacccttc

SEQ ID NO: 126 - DNA, 21 bp, artificial
  oligonucleotide primer
gagcagggcgtcatgcacaag

SEQ ID NO: 127 - DNA, 18 bp, artificial
  oligonucleotide primer
taggtggggacagacaat

SEQ ID NO: 128 - DNA, 21 bp, artificial
  oligonucleotide primer
aggtcggtgtgaacggatttg

SEQ ID NO: 129 - DNA, 23 bp, artificial
  oligonucleotide primer
tgtagaccatgtagttgaggtca

SEQ ID NO: 130 - protein, 12 aa, artificial
  tetra-amino acid repeat block
LAPCLAPCLAPC

SEQ ID NO: 131 - protein, 12 aa, artificial
  tetra-amino acid repeat block
APCLAPCLAPCL

SEQ ID NO: 132 - protein, 12 aa, artificial
  tetra-amino acid repeat block
PCLAPCLAPCLA

SEQ ID NO: 133 - protein, 12 aa, artificial
  tetra-amino acid repeat block
CLAPCLAPCLAP

SEQ ID NO: 134 - protein, 15 aa, artificial
  C-terminal digestion fragment
TTTTSSYPYDVPDYA

SEQ ID NO: 135 - protein, 14 aa, artificial
  polyA N-terminal peptide
AAAAAAAAAAAAAR

SEQ ID NO: 136 - protein, 4 aa, artificial
  tetra-amino acid repeat block
LAPC

SEQ ID NO: 137 - protein, 4 aa, artificial
  tetra-amino acid repeat block
QAGR

SEQ ID NO: 138 - DNA, 21 bp, artificial
  ATXN8 repeat portion of construct
aaaaagcagcggcagcggcag

SEQ ID NO: 139 - DNA, 21 bp, artificial
  ATXN8 repeat portion of construct
aaaatgcagcggcagcggcag

SEQ ID NO: 140 - DNA, 12 bp, artificial
  ATXN8 repeat portion of construct
taaaaaaagcag

SEQ ID NO: 141 - DNA, 30 bp, artificial
  repeat tract portion of construct
tagctaggtaactaagtaactagaatgcag

SEQ ID NO: 142 - DNA, 30 bp, artificial
  repeat tract portion of construct
tagctaggtaactaagtaactagaattcag

SEQ ID NO: 143 - DNA, 26 bp, artificial
  repeat tract portion of construct
tagctaggtaactaagtaactagcag

SEQ ID NO: 144 - DNA, 33 bp, artificial
  repeat tract portion of construct
tagctaggtaacttagtaactagaaattaagca

SEQ ID NO: 145 - DNA, 31 bp, artificial
  repeat tract portion of construct
tagctaggtaactaagtaactagaatagagc

SEQ ID NO: 146 - DNA, 30 bp, artificial
  repeat tract portion of construct
tagctaggtaactaagtaactagaatgcaa

SEQ ID NO: 147 - DNA, 30 bp, artificial
  repeat tract portion of construct
tagctaggtaactaagtaactagaattcaa

SEQ ID NO: 148 - protein, 6 aa, artificial
  repeat region of polypeptide
MQRQRQ

SEQ ID NO: 149 - protein, 11 aa, artificial
  repeat region of polypeptide
AVVVVKPGFLT

SEQ ID NO: 150 - DNA, 33 bp, artificial
  repeat region of construct
tagctaggtaactaagtaactagaatagagcag

SEQ ID NO: 151 - DNA, 23 bp, artificial
  repeat region of construct
taatatattttaaaaaaatgcag

SEQ ID NO: 152 - DNA, 23 bp, artificial
  repeat region of construct
taatatattttaaaaaaaagcag

SEQ ID NO: 153 - DNA, 23 bp, artificial
  repeat region of construct
tcgagtccctcaagtccttccag

SEQ ID NO: 154 - DNA, 23 bp, artificial
  repeat region of construct
cctgcacagaaaccatcttacag

SEQ ID NO: 155 - DNA, 23 bp, artificial
  repeat region of construct
aacagcagcaaaagcagcaacag

SEQ ID NO: 156 - DNA, 23 bp, artificial
  repeat region of construct
gaaatggtctgtgatccccccag

SEQ ID NO: 157 - DNA, 29 bp, artificial
  repeat portion of construct
agctaggtaactaagtaactagaattcag

SEQ ID NO: 158 - DNA, 29 bp, artificial
  repeat portion of construct
agctaggtaactaagtaactagaactcag

SEQ ID NO: 159 - DNA, 30 bp, artificial
  repeat portion of construct
agaatttcctgcacagaaaccatcttacag

SEQ ID NO: 160 - DNA, 30 bp, artificial
  repeat portion of construct
agaatttcctgcacagaaaccaccttacag

SEQ ID NO: 161 - DNA, 30 bp, artificial
  repeat portion of construct
agaacttcctgcacagaaaccatcttacag

SEQ ID NO: 162 - DNA, 30 bp, artificial
  repeat portion of construct
agaacttcctgcacagaaaccaccttacag

SEQ ID NO: 163 - DNA, 29 bp, artificial
  repeat portion of construct
agaatttcgagtccctcaagtccttccag

SEQ ID NO: 164 - DNA, 29 bp, artificial
  repeat portion of construct
agaacttcgagtccctcaagtccttccag

SEQ ID NO: 165 - DNA, 29 bp, artificial
  repeat portion of construct
agaattaacagcagcaaaagcagcaacag

SEQ ID NO: 166 - DNA, 29 bp, artificial
  repeat portion of construct
agaactaacagcagcaaaagcagcaacag

SEQ ID NO: 167 - DNA, 29 bp, artificial
  repeat portion of construct
agaattgaaatggtctgtgatccccccag

SEQ ID NO: 168 - DNA, 17 bp, artificial
  repeat portion of construct
aatttcagcgcgcgcag

SEQ ID NO: 169 - DNA, 23 bp, artificial
  repeat portion of construct
gtaactaagtaactagaattcag

SEQ ID NO: 170 - DNA, 23 bp, artificial
  repeat region of construct
cctgcacagaaaccatcttacag

SEQ ID NO: 171 - DNA, 23 bp, artificial
  repeat region of construct
cctgcacagaaaccatcttacag

(19) United States
(12) Patent Application Publication
(10) Pub. No.: US 2013/0115603 A9
(43) Pub. Date: May 9, 2013

(54) NUCLEOTIDE REPEAT EXPANSION-ASSOCIATED POLYPEPTIDES AND USES THEREOF

(71) Applicants: Laura P.W. Ranum, St. Paul, MN (US); Tao Zu, Shoreview, MN (US)

(72) Inventors: Laura P.W. Ranum, St. Paul, MN (US); Tao Zu, Shoreview, MN (US)

(73) Assignee: Regents of the University of Minnesota, Saint Paul, MN (US)

(21) Appl. No.: 13/262,477

(22) PCT Filed: Apr. 1, 2010
(86) PCT No.: PCT/US10/29673

§ 371 Date: Dec. 21, 2011

Related U.S. Application Data
(60) Provisional application No. 61/165,967, filed on Apr. 2, 2009.

(51) Int. Cl. G01N 33/566; C12Q 1/68; C07H 21/04; G01N 21/64; C07K 14/00; C07K 16/00

(52) U.S. Cl. 435/6.12; 530/350; 530/387.9; 536/23.1; 436/501; 435/7.1

(57) ABSTRACT

Isolated polypeptides that are endogenously expressed from nucleotide repeat expansions are disclosed. In some cases, the polypeptides include polypeptide repeats. In some cases, the polypeptide repeats include at least five contiguous repeats of a single amino acid. In other cases, the repeats include at least six contiguous amino acids of a tetra- or penta-amino acid repeat block.

### CROSS-REFERENCE TO RELATED APPLICATION

This application claims priority to U.S. Provisional Patent Application Ser. No. 61/165,967, filed Apr. 2, 2009.

### GOVERNMENT FUNDING

The present invention was made with government support under Grant Nos. P01NS058901 and R01NS040389, awarded by the National Institutes of Health. The Government has certain rights in this invention.

### BACKGROUND

A variety of neurodegenerative diseases are caused by microsatellite repeat expansions. Repeat expansions located within or outside ATG-initiated open reading frames (ORFs) are thought to cause disease by protein gain- or loss-of-function mechanisms or by RNA gain-of-function effects.

The polyglutamine (polyQ)-expansion diseases include Huntington disease (HD), dentatorubral-pallidoluysian atrophy (DRPLA), spinal and bulbar muscular atrophy (SBMA), and spinocerebellar ataxia types 1, 2, 3, 6, 7, and 17. Since these CAG.CTG expansion mutations were discovered, efforts to understand disease mechanisms have focused on elucidating the molecular effects of these proteins. While these polyQ-expansion proteins bear no homology to each other apart from the polyQ tract, a hallmark of these diseases is protein accumulation and aggregation in nuclear or cytoplasmic inclusions. Although the polyQ-expansion proteins are widely expressed in the CNS and other tissues, only certain populations of neurons are vulnerable in each disease.

The myotonic dystrophies (DM1 and DM2) are the best characterized examples of RNA-mediated expansion disorders. The mutation causing DM1 is a CTG repeat expansion in the 3′ untranslated region (UTR) of the dystrophia myotonica-protein kinase (DMPK) gene. Although DM1 can be clinically more severe than DM2, the discovery of the DM2 mutation and several mouse models provide strong support that many features of these diseases result from RNA gain-of-function effects in which the dysregulation of RNA-binding proteins is mediated by the expression of CUG and CCUG expansion transcripts. Additionally, RNA gain-of-function effects have recently been reported for COG and CAG expansion RNAs.

SCA8 is a dominantly inherited spinocerebellar ataxia caused by a CTG.CAG expansion. The mutation is bidirectionally transcribed in the CUG (AXN8OS) and CAG (ATXN8) directions and the CAG expansion transcripts express a nearly pure polyQ-expansion protein. These data suggest that both RNA and protein gain-of-function effects may be involved in SCA8. These results and additional reports of bidirectional expression across CTG.CAG and CCG.GCC repeat expansions at the DM1 and FMR1 loci, and throughout much of the genome, suggest that there are additional fundamental lessons to learn about how microsatellite expansion mutations are expressed and how these mutations cause disease.

### SUMMARY OF THE INVENTION

In one aspect, the invention provides an isolated polypeptide. Generally, the isolated polypeptide includes at least six contiguous amino acids of a RAN-translated polypeptide, wherein the six contiguous amino acids include at least six contiguous amino acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ NO:9, SEQ ID NO:10, SEQ ID NO:11; at least six contiguous amino acids of the N-terminal sequence of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96; or at least six contiguous amino acids of the C-terminal sequence of any one or more of SEQ ID NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ ID NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97.

In another aspect, the invention provides an isolated polypeptide that generally includes a repeat portion comprising at least five contiguous amino acids; and a non-repeat portion that includes at least six contiguous amino acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11; at least six contiguous amino acids of an N-terminal sequence of a RAN-translated polypeptide; and/or at least six contiguous amino acids of an C-terminal sequence of a RAN-translated polypeptide.

If the repeat portion comprises at least five contiguous repeated leucine residues, the second portion can include at least at least six contiguous amino acids of an amino acid sequence selected from SEQ ID NO:1 and SEQ ID NO:8.

If the repeat portion comprises at least five contiguous repeated alanine residues, the second portion can include at least six contiguous amino acids of an amino acid sequence selected from SEQ ID NO:2, SEQ ID NO:4, and SEQ ID NO:7.

If the repeat portion comprises at least five contiguous repeated serine residues, the second portion can include at least six contiguous amino acids of an amino acid sequence selected from SEQ ID NO:3 and SEQ ID NO:6.

If the repeat portion comprises at least five contiguous repeated glutamine residues, the second portion can include at least six contiguous amino acids of SEQ ID NO:5.

If the repeat portion comprises at least five contiguous repeated cysteine residues, the second portion can include at least six contiguous amino acids of SEQ ID NO:9.

If the repeat portion comprises at least five contiguous amino acids of SEQ ID NO:12 or at least six contiguous amino acids of SEQ ID NO:12, the second portion can include at least six contiguous amino acids of SEQ ID NO:10 or at least six contiguous amino acids of SEQ ID NO:11.

In another aspect, the invention includes an isolated polypeptide that includes at least six contiguous amino acids of the amino acid sequence depicted in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13.

In another aspect, the invention provides an isolated polynucleotide encoding an isolated polypeptide described herein.

In another aspect, the invention provides an antibody composition that specifically binds to a polypeptide described herein.

In another aspect, the invention provides a method of identifying a subject at risk for a condition characterized by a repeat expansion. Generally, the method includes receiving a biological sample from a subject, detecting whether the biological sample comprises a RAN-translated polypeptide associated with a condition characterized at least in part by a nucleotide repeat expansion, and identifying the subject as at risk for a condition characterized by a repeat expansion if the biological sample includes the RAN-translated polypeptide.

In some embodiments, detecting whether the biological sample comprises a RAN-translated polypeptide associated with a condition characterized at least in part by a nucleotide repeat expansion comprises contacting at least a portion of the biological sample with an antibody that specifically binds to a RAN-translated polypeptide and determining whether the antibody specifically binds to a component of the biological sample.

In another aspect, the invention provides a method of monitoring the presence and/or amount of a biomarker of a condition characterized by a repeat expansion. Generally, the method includes receiving a biological sample from a subject being treated for a condition characterized at least in part by a repeat expansion, measuring the amount of at least one biomarker indicative of a repeat expansion in the biological sample, and quantifying any change in the amount of biomarker in the sample with respect to a reference value of the amount of biomarker in a sample obtained prior to the subject being treated for the condition.

In some embodiments, the method further includes modifying the treatment if the change in the biomarker is less than a standard value indicative of efficacious treatment.

In another aspect, the invention provides a method for analyzing a subject's risk for developing a condition characterized at least in part by a nucleotide repeat expansion. Generally, the method includes receiving at least a first biological sample and a second biological sample from a subject, wherein at least one of the following is true: the first biological sample and the second biological sample were obtained from the subject at different times, or the first biological sample and the second biological sample were obtained from different tissues; measuring the amount of at least one biomarker indicative of a repeat expansion in each of the biological samples; and identifying any difference in the biomarker between the first biological sample and the second biological sample.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1: Non-ATG translation of ATXN8-CAG_{EXP} constructs (SEQ ID NO:138, SEQ ID NO:139, SEQ ID NO:140) generates polyQ, polyA, and polyS proteins in HEK293 cells. A) Immunoblot of protein lysates (right) from cells transfected with A8 minigenes (left) with endogenous 3′ sequence (A8-endo) and without an ATG start codon shows expression of ataxin-8 polyQ protein as a dark band at ˜40 kDa. The faint 40 kDa background band recognized by the 1C2 antibody in HEK293 cells transfected with empty vector (pcDNA3.1) results from reaction of antibody with the endogenous human TATA-binding protein (TBP), which contains ˜40 glutamines. *=stop codon, K=lysine, Q=glutamine, M=methionine. B) Modified A8 constructs with upstream 6× STOP codon cassette and with 3′ epitope tags in each frame [A8(*KKQ_{EXP})-3Tf1] and in staggered frames for A8(*KKQ_{EXP})-3Tf2 and A8(*KKQ_{EXP})-3Tf3 to allow detection of polyA, polyQ and polyS with the HA tag. Right, immunoblots of A8(*KKQEXP)-3Tf1 lysates probed with 1C2, α-His, α-myc, α-HA and α-Flag antibodies before and after treatment with Proteinase-K, DNase I and RNase I. Immunoblots of A8(*KKQEXP)-3Tf1, A8(*KKQ_{EXP})-3Tf2 and A8(*KKQ_{EXP})-3Tf3 lysates probed with α-HA show relative levels of polyS, polyQ and polyA proteins. The “f1”, “f2” and “f3” designations indicate 3′ tags have been shifted in the A8(*KKQ_{EXP})-3T constructs so that the HA tag is in the polyA, polyQ or polyS frame, respectively. C) Immunoblots of A8(*KKQ_{EXP})-3Tf1 lysates probed with 1C2, α-HA and α-Flag antibodies in cells treated with or without cycloheximide. The presence of an ATG start codon in the polyQ frame results in the generation of an additional polyQ band. Additionally, this sequence change also affects the migration pattern of the polyA protein and the relative levels of the polyS protein.

FIG. 2: RAN-translation depends on repeat length and hairpin structure. A)

Immunoblot detection of polyQ, polyA, and polyS proteins in HEK293 cells transfected with A8(*KKQ_{EXP})-3Tf1 or A8(*KMQ_{EXP})-3Tf1 constructs containing varying CAG repeat lengths (SEQ ID NO:141, SEQ ID NO:142, SEQ ID NO:143, SEQ ID NO:144, SEQ ID NO:145, SEQ ID NO:146, SEQ ID NO:147). B) Immunoblot detection of polyQ protein from cells transfected with ATT(CAG_{EXP})-3T constructs containing 105 or 52 but not 15 CAG repeats. C) Schematic diagram and protein blots showing protein expression from constructs with and without stop codons immediately preceding pure CAG, GCA, and AGC repeats. All four constructs contain 3′ epitope tags: myc-His (polyQ), HA (polyA), and Flag (polyS). Protein blots from transfected cells probed with 1C2, α-HA or α-FLAG antibodies. D) Triply-tagged constructs containing a CAA or CAG repeat tract with or without an ATG start codon in glutamine frame and immunoblot detection of polyQ proteins from transfected cells.

FIG. 3: RAN-translation in ATG-initiated ORF can occur in the absence of frame shifting. A) Diagram of constructs containing 5′ V5 epitope in the glutamine frame and 3′ Flag (Ser), HA (Ala), myc-His (Gln) epitope tags. B) Protein blots of cells transfected with +ATG construct in (A) probed with 1C2 and epitope antibodies. C) Protein blots of cells transfected with + and − ATG constructs probed with 1C2, α-V5 and α-myc antibodies.

FIG. 4: RAN translation across CUG expansion transcripts. A) Diagram of CTG containing constructs containing a myc/His tag in the polyC, polyA, or polyL frame. B) Immunoblot showing that polyC, polyA, and polyL can be made via RAN translation. Note that all of these homopolymeric proteins run as high molecular-weight smears.

FIG. 5: In vivo evidence for RAN-translated polyA protein (SCA8_{GCA-Ala}) in SCA8 mice and human samples. A) Diagram showing ATXN8 CAG transcript, ATG-initiated polyQ ORF, and putative non-ATG SCA8_{GCA-Ala} protein; *=stop codon (SEQ ID NO:148). The predicted gene-specific C-terminal protein sequence underlined in the alanine frame was used to generate SCA8_{GCA-Ala} peptide and α-SCA8_{GCA-Ala} polyclonal antibody (SEQ ID NO:149). B)α-SCA8_{GCA-Ala} antibody detects recombinant protein expressed in HEK293 cells transfected with the A8(*KMQ_{EXP})-endo minigene but not empty vector by protein blot and immunofluorescence. C) Top and Middle Panels: Immunohistochemical staining of cerebellar tissue using α-SCA8_{GCA-Ala} polyclonal antibody shows consistent staining of Purkinje cell bodies and dendrites in BAC SCA8 mice, but not non-transgenic littermates.

Lower Panels: Immunofluorescence staining of cerebellar tissue using α-SCA8_{GCA-Ala} polyclonal antibody shows staining (red-cy3) in Purkinje cells of BAC SCA8 mice, but not non-transgenic littermates. D) α-SCA8_{GCA-Ala} antibody shows specific staining (red-cy3) of human SCA8 but not control Purkinje cell which is distinct from occasional punctate background autofluorescence (positive in red, blue and open green channels). Co-labeling with α-PKCγ antibody (yellow-cy5) independently stains Purkinje cell bodies and confirms their presence in both the SCA8 and control sample.

FIG. 6: In vivo evidence for RAN-translated polyQ protein (DM1_{CAG-Gln}) in DM1. A) Diagram showing the antisense transcript of the DM1 CAG expansion and the predicted non-ATG initiated polyQ protein, *=stop codon. Predicted gene-specific C-terminal sequence in glutamine frame used to generate a DM1_{CAG-Gln} peptide and polyclonal antibody is underlined. B) α-DM1_{CAG-Gln} antibody detects recombinant fusion protein in HEK293 cells transfected with a construct designed to express the C-terminal portion of the endogenous DM1 polyQ protein (CAG_{EXP}-DM1-3′) by protein blot and immunofluorescence. Immunofluorescence staining of cardiomyocytes (C, D) and leukocytes (E) using α-DM1_{CAG-Gln} (cy3-red) in DM1 mice containing 55, 328 and >1000 CTG repeats but not in control mice. Round leukocytes in coagulated blood within heart chambers show positive staining with α-DM1_{CAG-Gln} for DM300 but not DM20 with comparable (non-serial) H&E sections on right. F) HRP labeled 1C2-positive cytoplasmic stain (blue) in leukocytes of DM55 but not DM20 control mouse. G) Co-localization of α-DM1_{CAG-Gln} (cy3-red) with caspase-8 (Alexa Fluor 488-green) in mouse cardiomyocytes. H) Staining with α-DM1_{CAG-Gln} (cy3-red) in human DM1 but not control leukocytes. I) Protein blots show a ˜55 kDa protein is detected from DM1 human peripheral blood with both the 1C2 and α-DM1_{CAG-Gln} antibodies.

FIG. 7: Polysome profiling, protein labeling and mass spectrometry. A) Polyribosome profiles from HEK293 cells transfected with (CAG_{EXP})-3T constructs (top) with (SEQ ID NO:141) or without (SEQ ID NO:150) an ATG initiation codon. Middle panels show the O.D. 254 with ribosomal subunit (40S and 60S), monosome (80S) and polysomal fractions indicated; corresponding RNA blots showing relative levels of CAG and GAPDH transcripts are shown in the lower panels. B) Protein blot (upper panel) and fluorograph (lower panel) proteins labeled with [³H]-Q, [³H]-A, or [³H]-S after IP with α-HA tag in HEK293 lysates transfected with A8(*KKQ_{EXP})-3Tf1, A8(*KKQ_{EXP})-3Tf2, A8(*KKQ_{EXP})-3Tf3 or empty vector. C) Representative identified spectrum of the predicted polyA N-terminal peptide AAAAAAAAAAAAAR (SEQ ID NO:135). Matched b-ions are shown in light shading and y-ions are shown in dark shading for the product ions of the associated precursor ion.

FIG. 8: Lenti-viral expansion constructs. Schematic diagram showing triply tagged lentiviral constructs used for infection of HEK293 cells mouse brains.

FIG. 9: Non-ATG translation of polyQ can be influenced by the length of CAG repeat tracts. A) Schematic diagram showing constructs in which stop codons were placed prior to pure CAG repeats and each of three frames was tagged with myc-His, HA, and Flag tags, respectively. B) Western blots showing constructs containing 105 or 52 CAG repeats, but not 15 repeats, express polyQ proteins.

FIG. 10: Cardiac histology in DM1 mice. H&E staining of cardiac tissue comparable to that used in FIG. 6C shows typical cardiac histology including large, boxy, centrally-located myocyte nuclei in both DM300 and WT samples.

FIG. 11: A) Constructs with 5′ flanking sequence from the HD, HDL2, DM1, and SCA3 loci and 3′ epitope tags (SEQ ID NO:151, SEQ ID NO:152, SEQ ID NO:153, SEQ ID NO:154, SEQ ID NO:155, SEQ ID NO:156). B) Protein blots after coupled in vitro transcription-translation of constructs in (A) using rabbit reticulocyte lysates. Blots are probed with 1C2, α-HA or α-FLAG antibodies.

FIG. 12: RAN translation in cell free RRLs is less permissive and requires alternative start codons. A) Protein blots after coupled in vitro transcription-translation of constructs in (FIG. 13B) using rabbit reticulocyte lysates (RRL). B) Schematic diagrams of repeat constructs with and without ATT or ATC alternative start codons in the Gln (Gln-f) or Ser (Ser-f) frames respectively (SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167). C) Protein blots of samples prepared using an in vitro RRL transcription/translation reaction (upper panel) or from transfected HEK293 cells (lower panel). D) Protein blots from RRL (upper panel) and HEK293 cells transfected with HD-3T, SCA3-3T and DM1-3T constructs (lower panel).

FIG. 13: RAN-translation occurs in various disease relevant sequence contexts and is sufficient to cause toxicity. A) RAN-translation in ATG-initiated ORF. Diagram of constructs containing 5′ V5 epitope in the glutamine frame and distinct 3′ epitope tags with and without a 5′ATG. Corresponding protein blots of cells transfected with (+) and without (−) ATG constructs probed with α-V5, 1C2, α-HA and α-FLAG antibodies. B) Constructs with 20 nt of 5′ flanking sequence upstream of repeat for transcripts expressed in the CAG direction at the HD, HDL2, DM1, and SCA3 loci and 3′ epitope tags and corresponding protein blots from transfected cells probed with 1C2, α-myc, α-HA or α-FLAG antibodies. C) Relative PI and annexin V positive N2a cells after transfection with ATG(CAA₉₀)-3T, ATT(CAG₁₀₅)-3T, ATG(CAG₁₀₅)-3T plasmids, relative to the negative homopolymeric protein control ATT(CAA₉₀)-3T with or without an ATG. (**): p<0.01 and (***):p<0.001. The corresponding immunoblots (right panel) show the relative levels of polyQ, polyA and polyS expressed in each transfection.

FIG. 14: Cellular expression of RAN translation products. Immunofluorescence staining of tagged polyQ (α-His/cy3), polyA (α-HA/cy5) and polyS (α-FLAG/FITC) proteins in cells transfected with A8(*KKQ_{EXP})-3Tf1. Scale bar=20 μm.

FIG. 15: Non-ATG translation in transfected and infected cells/tissues and rabbit reticulocyte lysates. A) Schematic diagram showing constructs with and without stop codons immediately preceding pure CAG, GCA, and AGC repeats and 3′ epitope tags: myc-His (Gln), HA (Ala), and Flag (Ser) (SEQ ID NO:142, SEQ ID NO:143, SEQ ID NO:144, SEQ ID NO:145). B) Protein blots from cells transfected with the constructs in (A) probed with 1C2, α-HA or α-FLAG antibodies.

FIG. 16: Semiquanitative RT-PCR of CAG and CAA transcripts. A) Schematic diagram depicting the RT-PCR strategy. The Myc RT Primer was used in a first strand synthesis reaction while the 336 F and 336 R primers were used for subsequent amplification over the repeat. B) RT-PCR results for the CAG and CAA repeat constructs and β-actin control in the presence (+) or absence (−) of reverse transcriptase (RT).

FIG. 17: Identification of N-terminal peptides of the polyA protein by tandem MS. A) Schematic diagram showing the construct containing CGCGCG interruption (upper panel) (SEQ ID NO:168) and the predicted sequence of the polyA with the inserted R and C-terminal HA tag (lower panel). B) N-terminal polyA peptides are identified containing varying numbers of alanine [(A)₉₋₁₈R].

FIG. 18: Representative identified spectrum of polyA C-terminal peptide TTTTSSYPYDVPDYA (SEQ ID NO:134). Matched b-ions are shown in red and y-ions are shown in blue for the product ions of the associated precursor ion. Below each spectrum are fragmentation tables displaying matched product ions. The precursor ion was +2 charged with a mass error of −0.32 ppm. The SEQUEST Xcorr and deltaCN values were 2.59 and 0.42. More than 100 spectra with peptide probabilities at 95% were assigned to this protein from 2 separate IP experiments which included 12 unique peptides.

FIG. 19: RAN-translation in ATG-initiated ORF. Protein blots of HEK293 cells transfected with constructs in FIG. 4A after immunoprecipitation with antibodies to 3′ epitope tags in polyQ (α-His), polyA (α-HA), and polyS (α-Flag) frames probed for the 5′ epitope tag with α-V5 (top panel) or 1C2, α-HA, α-Flag (bottom panel). Right panel shows faint polyQ background band without IP, indicating similar staining in middle panels is caused by non-specific binding of polyQ to the beads.

FIG. 20: Non-AUG translation following RNA transfection into HEK293 cells. A) Non-ATG CAG expansion constructs (SEQ ID NO:169, SEQ ID NO:153. SEQ ID NO:170, SEQ ID NO:171) used to produce capped, polyadenylated mRNAs that extend from the T7 promoter to the PvuII site (P) where the plasmid was linearized (22 bp beyond the polyadenylation site. B) Immunoblot of HEK293 lysates following RNA transfections using constructs in panel A probed with 1C2 antibody.

FIG. 21: Non-ATG translation in infected cells and tissues. A) Schematic diagram showing triply tagged lentiviral constructs used for infection of HEK293 cells and mouse brains. All lentiviral constructs are in the CSII lentiviral vector. B) Protein blots of HEK293 cells after lentiviral vector infection with Lt-GFP, Lt-A8(*KMQ_{EXP})f1, Lt-HD, Lt-HDL2, Lt-SCA3, and Lt-DM1(M_{S}). Infected HEK293 cells show robust non-ATG translation of polyQ proteins for Lt-HDL2 and Lt-DM1. PolyA but not polyS is expressed from all four constructs (Lt-HD, Lt-HDL2, Lt-SCA3, and Lt-DM1) without an ATG in the polyA frame. C) Protein blots of mouse cerebellar extracts after lentiviral vector infection and immunoprecipitation. The ˜40 kDa 1C2-positive protein was detected in cerebellar lysates injected with Lt-A8(*KMQ_{EXP}), Lt-HDL2, and Lt-DM1(M_{S}), but not Lt-HD, Lt-SCA3, and Lt-GFP. Two FVB animals were injected with each of these viruses and four weeks post-injection, tagged-polyQ protein was immunoprecipitated with anti-His antibody and probed with 1C2. As shown in Supplemental FIG. 9C, tagged polyQ protein was immunoprecipitated from tissue infected with the +ATG control virus Lt-A8(*KMQ_{EXP}) as well as from tissue infected with the Lt-DM1 and Lt-HDL2 lacking an ATG in the glutamine frame, although at a substantially lower level.

FIG. 22: Fluorograph (top panel) showing [³⁵S]-methionine incorporation and protein blot (lower panel) of the same in vitro translation products probed with the 1C2 antibody.

FIG. 23: In situ hybridization of CAG probe to detect CUG-containing RNA foci in cardiac sections from DMSXL and DM20 control (right) animals.

FIG. 24: RT-PCR analysis of CAG DMPK antisense transcripts. A) Diagrams showing DMPK 3′ UTR and location of antisense specific primers for the CAG transcript. For strand-specific priming, a linker sequence (lk) was attached to the DM1-specific primers for cDNA synthesis (lk-1 or lk-2). PCR was performed using a primer complementary to the lk sequence and reverse primers anti1B, antiN3 or antiA2. The 3′ end of the DM1 CAG RNA is unknown. B) Strand-specific RT-PCR of the human DMPK antisense strand in transgenic mice. Strand-specific reverse transcription and PCR were performed with RNA from a pool of 5 month-old mouse hearts (n=3) and with RNA from DM1 and control human heart samples. Various lines of transgenic mice have been assessed: DM20 mice with 20 CTGs, DM55 with 55 CTGs, DM300 with ˜300 CTGs, DMSXL with >1000 CTGs. M: 250 bp DNA ladder, wt ms=wild type mouse, DM1 hs heart=DM1 human heart, Ctrl hs heart=human control heart, and heterozygous and homozygous DM mice. Asterisks to the right of corresponding lanes indicate PCR products with large repeats that amplified with low efficiency. Primers used for DNA synthesis and for PCR are indicated on the left. Gapdh indicates PCR with primers for the mouse Gapdh cDNA that self primed during reverse transcription. Note that these primers also amplified endogenous human GAPDH cDNA, at lower efficiency.

FIG. 25: DM1 polyQ protein co-expressed with caspase 8 in human skeletal muscle. A) Staining with α-DM1_{CAG-Gln} (cy3-red) in human DM1 but not control skeletal muscle autopsy tissue. B) DM1 human longitudinal skeletal muscle section showing co-expression of polyQ (red) and caspase 8 (green). C) Staining with α-DM1_{CAG-Gln} (cy3-red) in DM1 but not control myoblasts.

### DETAILED DESCRIPTION OF ILLUSTRATIVE EMBODIMENTS

The present invention relates to polypeptides that have been discovered to be expressed in the absence of an AUG start codon from trinucleotide, tetranucleotide, or pentanucleotide repeats. Such repeats, and RAN-translated polypeptides encoded by such nucleotide repeats, are associated with certain neurodegenerative disorders such as, for example, myotonic dystrophy type 1 (DM1), myotonic dystrophy type 2 (DM2), spinocerebellar ataxia type 3 (SCA3), spinocerebellar ataxia type 8 (SCA8), Huntington Disease (HD), and others. Thus, detection of the polypeptides or detection of polynucleotides from which the polynucleotides are expressed may provide a method of detecting whether a subject possesses the nucleotide expansions associated with the identified and other neurodegenerative disorders.

In some embodiments, the isolated polypeptide can generally include a repeat portion comprising at least five contiguous amino acids and a second portion comprising at least six contiguous amino acids of a “non-repeat” amino acid sequence bearing a specified level of similarity and/or identity to an N-terminal sequence or a C-terminal sequence of a RAN-translated polypeptide.

The term “repeat portion” refers to a portion of a polypeptide that includes a repeating pattern of amino acids. In some cases, the repeat portion can include a homopolymeric repeat of a single amino acid (e.g, (A)ₙ, where A is alanine and n is the number of contiguously repeated amino acid residues). In other cases, the repeat portion can include the repeat of a contiguous block of amino acids such as, for example, a repeating four amino acid block—e.g., (LAPC)ₙ, where LAPC is a complete amino acid block that includes leucine, alanine, proline and serine, and n is the number of contiguous repeats of the four amino acid block.

The term “non-repeat” amino acid sequence refers to an amino acid sequence possessing a specified level of amino acid similarity and/or amino acid identity with a portion of a RAN-translated polypeptide that lacks a repeating pattern of at least five contiguous amino acids associated with RAN-translation. Repeat patterns—e.g., homopolymeric repeats and repeat blocks—associated with RAN-translation are described in more detail below.

As used herein, the term “polypeptide” refers to a polymer of amino acids linked by peptide bonds. Thus, for example, the terms peptide, oligopeptide, protein, and enzyme are encompassed within the definition of polypeptide. This term also includes post-expression modifications of the amino acid polymer such as, for example, glycosylations, acetylations, phosphorylations, and the like. The term polypeptide does not connote a specific length of a polymer of amino acids. A polypeptide may be isolatable directly from a natural source, or can be prepared with the aid of recombinant, enzymatic, or chemical techniques.

An “isolated” polypeptide is one that has been removed from its natural environment. For instance, an isolated polypeptide is a polypeptide that has been removed from the cytoplasm or from the membrane of a cell so that many of the polypeptides, nucleic acids, and other cellular material of its natural environment are no longer present. In some cases, an isolated polypeptide may be characterized by the extent to which it is removed from components with which it is naturally associated such as, for example, at least 60% free, at least 75% free, or at least 90% free from other components with which they are naturally associated. Polypeptides that are produced outside the organism in which they naturally occur, e.g., through chemical or recombinant means, are considered to be isolated by definition since they were never present in a natural environment.

The term “clinical sign” or, simply, “sign” refers to objective evidence of disease or condition.

The term “RAN-translation” refers to Repeat Associated Non-ATG translation, which refers to translation of a polypeptide initiated from an mRNA sequence other than a typical mRNA translation initiation AUG codon, which corresponds to an ATG codon in DNA.

The term “symptom” refers to subjective evidence of disease or condition experienced by the patient.

The term “and/or” means one or all of the listed elements or a combination of any two or more of the listed elements.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Polypeptides described herein can include a repeat portion and a second portion. If present, the repeat portion of the polypeptide includes an amino acid sequence that is a translation product of a nucleotide repeat such as, for example, a trinucleotide, tetranucleotide, or pentanucleotide repeat associated with a neurogenerative disease such as, for example, myotonic dystrophy type 1 (DM1), myotonic dystrophy type 2 (DM2), spinocerebellar ataxia type 3 (SCA3), spinocerebellar ataxia type 8 (SCA8), or Huntington Disease (HD). As noted above, RAN-translation of nucleotide repeats such as those just described can occur in a variety of disease-relevant sequence contexts, suggesting that this phenomenon may occur in a wide range of repeat diseases.

RAN-translation of a nucleotide repeat expansion has at least two consequences. One consequence is the expression of a polypeptide that includes a repeated amino acid block. The number of amino acids in a complete repeat block is determined by the number of nucleotides in the nucleotide repeat, as described in more detail below. Another consequence is that otherwise noncoding regions of mRNA are translated. Translation is initiated in the absence of an AUG start codon, continues through the nucleotide repeat expansion, and continues beyond the 3′ end of the nucleotide repeat expansion into otherwise untranslated sequences of the mRNA. Thus, RAN-translation can result in the translation of novel amino acid sequences encoded by the otherwise noncoding nucleotide sequences beyond the 3′ end of a nucleotide repeat expansion. In some instances, RAN-translation can be initiated upstream of the nucleotide repeat expansion so that otherwise untranslated sequences of the mRNA upstream of the 5′ end of the nucleotide repeat expansion are translated.

If the nucleotide repeat includes repetition of a trinucleotide block, the resulting translation product includes a contiguous repeat of a single amino acid. Depending upon the sequence of the specific trinucleotide repeat block and the frame in which translation initiates, as many as three different polypeptide repeats are possible from a given trinucleotide repeat block—i.e., as many as one different amino acid repeat for each of the three possible reading frames. For example, a (CAG) trinucleotide repeat block can be translated in each of three frames, each frame producing a different polypeptide repeat product: (CAG)ₙ is translated as polyglutamine (Q)ₙ, (AGC)ₙ is translated as polyserine (S)ₙ, and (GCA)ₙ is translated as polyalanine (A)ₙ.

If the nucleotide repeat includes a tetranucleotide block repeat, the resulting translation product will include a tetra-amino acid block repeat. For example, a (CAGG) nucleotide repeat block will be translated as a (QAGR) amino acid repeat block. Exemplary tetra-amino acid repeat blocks include LAPC and QAGR. Reference to an amino acid repeat block indicates the sequential order of the amino acid residues that compose a complete repeat block, but is not intended to connote a particular amino acid that must begin either the repeat block or the repeat portion of the polypeptide. Thus, reference to the tetra-amino acid repeat block LAPC can include polypeptides such as, for example, a polypeptide that begins with a leucine (e.g., H₂N-LAPCLAPCLAPC-OH) (SEQ ID NO:130), a polypeptide that begins with an alanine (e.g., H₂N-APCLAPCLAPCL-OH) (SEQ ID NO:131), a polypeptide that begins with a proline (e.g., H₂N-PCLAPCLAPCLA-OH) (SEQ ID NO:132), or a polypeptide that begins with a cysteine (e.g., H₂N-CLAPCLAPCLAP-OH) (SEQ ID NO:133). Thus, a repeat portion of a polypeptide described herein can include, for example, an amino acid sequence that includes at least five contiguous amino acids of either of SEQ ID NO:12 or SEQ ID NO:13.

In some cases, the nucleotide repeat expansion can cause a hairpin to form in transcribed mRNA and the hairpin so formed may promote initiation of RAN-translation.

When present, the repeat portion of the polypeptide can vary in length. One feature of nucleotide repeat expansions associated with the conditions described herein is that the nucleotide repeat expansions can vary in length. Consequently, the length of polypeptide produced RAN-translated from mRNA transcribed from a nucleotide repeat expansion can vary. In some cases, the length of the repeat portion is at least five amino acids such as, for example, at least six amino acids, at least seven amino acids, at least eight amino acids, at least nine amino acids, at least ten amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 40 amino acids, at least 50 amino acids, at least 100 amino acids, at least 150 amino acids, at least 200 amino acids, or at least 300 amino acids. In some cases, the length of the repeat portion is no more than 500 amino acids such as, for example, no more than 300 amino acids, no more than 150 amino acids, no more than 100 amino acids, no more than 50 amino acids, no more than 20 amino acids, no more than 15 amino acids, no more than 10 amino acids, no more than nine amino acids, no more than eight amino acids, no more than seven amino acids, no more than six amino acids, or no more than five amino acids.

In cases in which the repeat portion of the polypeptide includes contiguous repeats of a block (e.g., a tetra- or penta-amino acid block) amino acids, the repeat portion of the polypeptide need not include a whole number of complete amino acid repeat blocks. Thus, a repeat portion of a polypeptide can include, for example, a total of 11 amino acids representing two complete repeats of a tetra-amino acid repeat block and a partial—i.e., three out of four amino acids—third repeat of the block.

When present, the second, non-repeat portion of the polypeptide can be the natural product of translation upstream of the 5′ end of a nucleotide repeat expansion or the natural product of translation downstream of the 3′ end of a nucleotide repeat expansion. Thus, the non-repeat portion can include amino acids beyond the N-terminal end of the repeat portion of an endogenously expressed RAN-translated polypeptide, amino acids beyond the C-terminal end of the repeat portion of an endogenously expressed RAN-translated polypeptide, or both. Thus, the second, non-repeat portion of the polypeptide is sometimes referred to herein as an “N-terminal sequence” (e.g., amino acids 1-7 of SEQ ID NO:14), “C-terminal end” (e.g., the C-terminal end of the predicted putative ATXN8-GCA-encoded polyA shown in FIG. 5A, which includes SEQ ID NO:2), or “C-terminal sequence.” Moreover, the portion of an mRNA that encodes an N-terminal sequence or a C-terminal sequence may be separated from the nucleotide repeat expansion until the mRNA is spliced. In addition, current recombinant technology permits the design of polypeptides in which the position of amino acids sequences within the polypeptide may be rearranged such as, for example, creating a polypeptide in which an N-terminal sequence is located somewhere in the polypeptide other than the N-terminus and/or a C-terminal sequence is located somewhere in the polypeptide other than the C-terminus. Thus, reference to the second, non-repeat portion of the polypeptide as an “N-terminal end,” “N-terminal sequence,” “C-terminal end,” or “C-terminal sequence” refers only to its location relative to the repeat portion as endogenously expressed in a RAN-translated polypeptide and is not intended to require that the polypeptide necessarily includes a repeat portion, restrict the useful location of a non-repeat portion in a polypeptide of the present invention, or the precise proximity of the mRNA encoding the non-repeat portion to the nucleotide repeat expansion.

The second, non-repeat portion of the polypeptide can include at least six contiguous amino acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, the N-terminal sequence, as shown in Table 1, of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96, the C-terminal sequence, as shown in Table 1, of any one or more of SEQ ID NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ ID NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97. Moreover, a polypeptide of the invention can include any combination of two or more of the foregoing non-repeat portions.

When present, the second, non-repeat portion can vary in length. The length of an N-terminal sequence can be influenced by, for example, whether a RAN-translation site exists upstream of the nucleotide repeat expansion and, if present, its location with respect to the nucleotide repeat expansion. The length of a C-terminal sequence can be influenced by, for example, the location of a STOP codon with respect to the nucleotide repeat expansion in the RAN-translated reading frame. In some cases, the length of the second, non-repeat portion is at least six amino acids such as, for example, at least seven amino acids, at least eight amino acids, at least nine amino acids, at least ten amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 40 amino acids, at least 50 amino acids, at least 100 amino acids, at least 150 amino acids, at least 200 amino acids, or at least 300 amino acids. In some cases, the length of the repeat portion is no more than 500 amino acids such as, for example, no more than 300 amino acids, no more than 150 amino acids, no more than 100 amino acids, no more than 50 amino acids, no more than 20 amino acids, no more than 15 amino acids, no more than 10 amino acids, no more than nine amino acids, no more than eight amino acids, no more than seven amino acids, no more than six amino acids, or no more than five amino acids.

In some embodiments, the polypeptide of the invention need not include a repeat portion. In such embodiments, the polypeptide of the invention can include at least six contiguous amino acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, the N-terminal sequence, as shown in Table 1, of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96, the C-terminal sequence, as shown in Table 1, of any one or more of SEQ ID NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ ID NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97. Moreover, a polypeptide of the invention can include any combination of two or more of the foregoing non-repeat portions.

In such embodiments, the polypeptide can vary in length. In some cases, the length of the polypeptide is at least six amino acids such as, for example, at least seven amino acids, at least eight amino acids, at least nine amino acids, at least ten amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 40 amino acids, at least 50 amino acids, at least 100 amino acids, at least 150 amino acids, at least 200 amino acids, or at least 300 amino acids. In some cases, the length of the repeat portion is no more than 500 amino acids such as, for example, no more than 300 amino acids, no more than 150 amino acids, no more than 100 amino acids, no more than 50 amino acids, no more than 20 amino acids, no more than 15 amino acids, no more than 10 amino acids, no more than nine amino acids, no more than eight amino acids, no more than seven amino acids, no more than six amino acids, or no more than five amino acids.

As used throughout this disclosure, reference to the amino acid sequence, or any portion thereof, of a particular SEQ ID NO includes embodiments possessing a specified level of amino acid sequence similarity and/or identity with the particularly identified SEQ ID NO or the specified portion thereof. Amino acid sequence similarity or sequence identity is generally determined by aligning the residues of the two amino acid sequences (i.e., a candidate amino acid sequence and a reference amino acid sequence) to optimize the number of identical amino acids along the lengths of their sequences; gaps in either or both sequences are permitted in making the alignment in order to optimize the number of identical amino acids, although the amino acids in each sequence must nonetheless remain in their proper order. Reference amino acid sequences include the full amino sequence or any specified portion of, for example, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97.

A pair-wise comparison analysis of amino acid sequences can be carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. “Amino acid identity” refers to the presence of identical amino acids. “Amino acid similarity” refers to the presence of not only identical amino acids, but also the presence of conservative substitutions. A conservative substitution for an amino acid in a polypeptide of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2.

A candidate polypeptide can include an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

A candidate polypeptide can include an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

In embodiments without a repeat portion, a polypeptide of the present invention can include an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence such as, for example, any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, the N-terminal sequence, as shown in Table 1, of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96, the C-terminal sequence, as shown in Table 1, of any one or more of SEQ ID NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ ID NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97, or any combination of two or more such amino acid sequences.

In other embodiments without a repeat portion, a polypeptide of the present invention can include an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence such as, for example, any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, the N-terminal sequence, as shown in Table 1, of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96, the C-terminal sequence, as shown in Table 1, of any one or more of SEQ ID NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ ID NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97, or any combination of two or more such sequences.

In one aspect, the invention includes an antibody composition that can specifically bind to at least a portion of a polypeptide described herein. As used herein, an antibody that can “specifically bind” to at least a portion of a polypeptide is an antibody that interacts with the epitope of the polypeptide or interacts with a structurally related epitope. The antibody may specifically bind to a repeat portion of a polypeptide such as, for example, a portion of a (A)ₙ amino acid repeat, a portion of a (L)ₙ amino acid repeat, a portion of a (S)ₙ amino acid repeat, a portion of a (Q)ₙ amino acid repeat, a portion of a (C)ₙ amino acid repeat, a portion of a (LAPC)ₙ (SEQ ID NO:136) amino acid repeat, or a portion of a (QAGR)ₙ (SEQ ID NO:137) amino acid repeat. Alternatively, the antibody may specifically bind to a portion of an amino acid sequence that includes at least six contiguous amino acids from a non-repeat portion of a RAN-translated polypeptide. Exemplary polypeptides include, for example, any one of the amino acid sequences listed in Table 1.

TABLE 1

| Condition | Frame | Amino acid sequence | SEQ ID NO: |
|---|---|---|---|
| SCA8 | 5′ → 3′ | DNIFLKNAAAAAAAAAAAAAAAVV | SEQ ID NO: 14 |
| | Frame 1 | VVVVVVVVVKGFLT | |
| | 5′ → 3′ | QQQQQQQQQQQQQQQ | SEQ ID NO: 15 |
| | Frame 2 | | |
| | 5′ → 3′ | YIFKKCSSSSSSSSSSSSSSSSSSSSSSS | SEQ ID NO: 16 |
| | Frame 3 | SSKARFSNMKDPGSSQGIGNRASAN | |
| | | RVNLSVEAGSQKRQSECKDK | |
| | 3′ → 5′ | KTWLYYYYYYYYYYYCCCCCCCC | SEQ ID NO: 17 |
| | Frame 1 | CCCCCCCIF | |
| | 3′ → 5′ | SPIPNSLARPWVLHVRKPGFTTTTTT | SEQ ID NO: 18 |
| | Frame2 | TTTTTAAAAAAAAAAAAAAAFFKNI | |
| | | LSYFTI | |
| | 3′ → 5′ | LENLALLLLLLLLLLLLLLLLLLLLLL | SEQ ID NO: 19 |
| | Frame 3 | LLLLHFLKIYYLILLFDVIIVIYFSTLP | |
| | | HTAYLLLKNL | |
| DM1 | 5′ → 3′ | RPGREGPGPRPANGARRVLVAGNA | SEQ ID NO: 20 |
| | Frame 1 | AAAAGGITDHFFLSARLRP | |
| | 5′ → 3′ | LLLLLGGSQTISFFRPG | SEQ ID NO: 21 |
| | Frame 2 | | |
| | 5′ → 3′ | VPGARHRSRAHRLPVHNRSERGSPP | SEQ ID NO: 22 |
| | Frame 3 | SSSPVIRARPLAAGEGGAGSAAGER | |
| | | GSKGPCSRECCCCCWGDHRPFLSFG | |
| | | QAEALTWMGKLQAWEGSKPGRPCS | |
| | | ILHAPPPIVGSQSAKLSCA | |
| | 3′ → 5′ | VCDPPSSSSSIPGYKDPSSPVRRPRTR | SEQ ID NO: 23 |
| | Frame 1 | PLPPRPLGGGPGSQDWSWAETHARS | |
| | | GCELAGGGRGFCAVPRALSLPTGPR | |
| | | SRRQF | |
| | 3′ → 5′ | GGGRGIPEKAGLAKANFPSKQAEIAP | SEQ ID NO: 24 |
| | Frame 2 | DAPQSRASCTRKLCTLRTNDRWGC | |
| | | VEDGTRTARLAAFPGLQFAHPRQGL | |
| | | SLAERKKWSVIPPAAAAAFPATRTL | |
| | | RAPFAGRGPGPSLPGR | |
| | 3′ → 5′ | SPQQQQQHSRLQGPFEPRSPAADPAP | SEQ ID NO: 25 |
| | Frame 3 | PSPAARGRARITGLELGGDPRSERL | |
| DM2 | 5′ → 3′ | VLLPVCVCVCVCVCVCVCLSVCLSV | SEQ ID NO: 26 |
| | Frame 1 | CLSVCLPACLPACLPGCLSACLPACL | |
| | | PACLPVCLTLSPRLECSGMISAHCNL | |
| | | HPPGSSDSSASAS | |
| | 5′ → 3′ | VNEYYCQCVCVCVCVCVCVSVCLS | SEQ ID NO: 27 |
| | Frame 2 | VCLSVCLSACLPACLPACLAACLPA | |
| | | CLPACLPACLSVSLCPLGWSAVV | |
| | 5′ → 3′ | SITASVCVCVCVCVCVCLSVCLSVC | SEQ ID NO: 28 |
| | Frame 3 | LSVCLPACLPACLPAWLPVCLPACLP | |
| | | ACLPACLSHFVP | |
| | 3′ → 5′ | AEIIPLHSSLGDKVRQTGRQAGRQA | SEQ ID NO: 29 |
| | Frame 1 | GRQADRQPGRQAGRQAGRQTDRQT | |
| | | DRQTDRQTHTHTHTHTHTHTGSNTH | |
| | | SLIPSPT | |
| | 3′ → 5′ | DRQAGRQAGRQAGRQTGSQAGRQA | SEQ ID NO: 30 |
| | Frame 2 | GRQAGRQTDRQTDRQTDRHTHTHT | |
| | | HTHTHTLAVILIHSFQVQLNGHICMV | |
| | | IRP | |
| | 3′ → 5′ | TRGVEVAVSRDHTTALQPRGQSETD | SEQ ID NO: 31 |
| | Frame 3 | RQAGRQAGRQAGRQAARQAGRQA | |
| | | GRQADRQTDRQTDRQTDTHTHTHT | |
| | | HTHTHWQ | |
| HD | 5′ → 3′ | AAGTGPRWTAAQVLLLPAAQSPIHC | SEQ ID NO: 32 |
| | Frame 1 | PGAERRRESARGLRGLPCRAGDRHG | |
| | | DPGKADEGLRVPQVLPAAAAAAAA | |
| | | AAAAAAAAAAAAATAATAAAAAA | |
| | | ASSASSAAAAGTAAAASAAAAPAA | |
| | | APAATRPGCG | |
| | 5′ → 3′ | N/A | |
| | Frame 2 | | |
| | 5′ → 3′ | RPSSPSSPSSSSSSSSSSSSSSSSSSSSSS | SEQ ID NO: 33 |
| | Frame 3 | NSRHRRRRRRRLLSFLSRRRRHSRCC | |
| | | LSRSRPRRRPRRHPARLWLRSRCTD | |
| | | QRKNFQLPRKTV | |
| | 3′ → 5′ | GGGGGGGGGGGCCCCCCCCCCCCC | SEQ ID NO: 34 |
| | Frame 1 | CCCCCCCCCWKDLRDSKAFISFSRV | |
| | | AMAVSRPARQSPEASGRLAAPLSTG | |
| | | AMNGALGRR | |
| | 3′ → 5′ | GSGAEVGEGLAPGGGGCPSWALGC | SEQ ID NO: 35 |
| | Frame 2 | WVTLSLRGRGFVSPARRLQGYRHPR | |
| | | RSLGPAGTGSCSGPKLTVGAAAPQP | |
| | | QPGRVAAGAAAGAAAAEAAAAVP | |
| | | AAAAEEAEEAAAAAAAVAAVAAA | |
| | | AAAAAAAAAAAAAAAAGRT | |
| | 3′ → 5′ | SVQRLLSHSRAGWRRGRRRGRLRLR | SEQ ID NO: 36 |
| | Frame 3 | QQRLCLRRRLRKLRRRRRRRRRWR | |
| | | LLLLLLLLLLLLLLLLLLLLLLLEGLE | |
| | | GLEGLHQLFQGRHGGLPPGTAVPGG | |
| | | LGPTRGAAQHRGNEWGSGPQVKAE | |
| | | PERPSILDPSRQPPRRLASQTLRRRRR | |
| | | GRAGGGGATPASMIDSPSLRTLPMA | |
| | | GQGTSPPLPPQVLPHTARPLTAQRPT | |
| | | RAKARGSTERGRGVVRL | |
| HDL2 | 5′ → 3′ | RVRCTEEWISESPGRRAAAEPAKVP | SEQ ID NO: 37 |
| | Frame 1 | CTETILQQQQQQQQQQQQQQQQAA | |
| | | AAAAAAAAAAAAAAAAAAAAAAA | |
| | | AAAAAAAAAAAAAAGSSLASGPGS | |
| | | APNAVAS | |
| | 5′ → 3′ | APTWEWTGRGSQFVWGLASRKGPA | SEQ ID NO: 38 |
| | Frame 2 | PCVSGALRSGYRRVQAGGQLQSRPR | |
| | | FPAQKPSYSSSSSSSSSSSSSSSRQQQ | |
| | | QQQQQQQQQQQQQQQQQQQQQQQ | |
| | | QQQQQQQQQQQQQAAPWLPAPALP | |
| | | RMRWHLRMKAQIDSLN | |
| | 5′ → 3′ | GVDIGESRQAGSCRAGQGSLHRNHL | SEQ ID NO: 39 |
| | Frame 3 | TAAAAAAAAAAAAAAAGSSSSSSSS | |
| | | SSSSSSSSSSSSSSSSSSSSSSSSSSSSSS | |
| | | RQLPGFRPRLCPECGGILE | |
| | 3′ → 5′ | LRESICAFILRCHRIRGRAGAGSQGA | SEQ ID NO: 40 |
| | Frame 1 | ACCCCCCCCCCCCCCCCCCCCCCCC | |
| | | CCCCCCCCCCCCCCCLLLLLLLLLLL | |
| | | LLLLL | |
| | 3′ → 5′ | DATAFGAEPGPEARELPAAAAAAAA | SEQ ID NO: 41 |
| | Frame 2 | AAAAAAAAAAAAAAAAAAAAAAA | |
| | | AAAAAAAACCCCCCCCCCCCCCCC | |
| | | KMVSVQGTLAGSAAARLPGLSDIHS | |
| | | SVHLTRMEPVLSWKPDPKQTGFPDQ | |
| | | STPMWELILRGTGSSASLGRLACFRH | |
| | | GCLGRE | |
| | 3′ → 5′ | PPHSGQSRGRKPGSCLLLLLLLLLLL | SEQ ID NO: 42 |
| | Frame 3 | LLLLLLLLLLLLLLLLLLLLLLLLLLL | |
| | | LPAAAAAAAAAAAAAAAVRWFLC | |
| | | REPWPALQLPACLDSPISTPQCT | |
| SCA12 | 5′ → 3′ | SSSSSSSSSSCECARVGVRVSALAPA | SEQ ID NO: 43 |
| | Frame 1 | AAPCPAPRQLPYPRLPEPPSRGTSTLI | |
| | | PARQA | |
| | 5′ → 3′ | CTSRLQPPAAAAAAAAAAAASARV | SEQ ID NO: 44 |
| | Frame 2 | WV | |
| | 5′ → 3′ | TCKLVACCPGADSRLHAPHCSKEQP | SEQ ID NO: 45 |
| | Frame 3 | QPLPRPPLLLGKSRGAADAVGRSLA | |
| | | FNAPAASSLLQQQQQQQQQQLRVR | |
| | | ACGCEGECAGAGCSALPSSPPASLPP | |
| | | PAGAALPWDQHPHSGQASLNPVPAI | |
| | | SPLQLGSRKAPFCRGCPLSQGEEGSF | |
| | | LHFGAAAKEGNLLGISPDPLVSASG | |
| | | AGGRDQTPRGTAYLGTRRQRRQAQ | |
| | | HPRWELELE | |
| | 3′ → 5′ | VPFWTRAAVARWQGQDSGLPGRNE | SEQ ID NO: 46 |
| | Frame 1 | GAGPTGGRLRQAGVGKLAGSWAGR | |
| | | CSRRQRTHPHTHTRALAAAAAAAA | |
| | | AAAAGGWRRLVH | |
| | 3′ → 5′ | GSWRGAGQGAAAGASALTLTPTRA | SEQ ID NO: 47 |
| | Frame 2 | HSQLLLLLLLLLLQEAGGGWCIKGE | |
| | | APPNRVSSPTTLSQQQWWPRQRLRL | |
| | | LFAAVGRMQPRIGTWAASD | |
| | 3′ → 5′ | GCWSHGRAAPAGGGREAGGELGRA | SEQ ID NO: 48 |
| | Frame 3 | LQPAPAHSPSHPHARTRSCCCCCCC | |
| | | CCCRRLEAAGALKARLLPTASAAPR | |
| | | LFPSSSGGRGRGCGCSLLQWGACSR | |
| | | ESAPGQQATSLQVQVERIDALFQPPP | |
| | | LSRHSAKGHVYASTQSP | |
| Fragile X- | 5′ → 3′ | TASAGGGGDGGAAARGRAAARRRR | SEQ ID NO: 49 |
| associated | Frame 1 | RRRRRRRRRRRRRRRRLGLERPQPT | |
| conditions | | SRGRAPGASRAEEK | |
| | 5′ → 3′ | RRARAAAVTEAPLPGGVRQRGGGG | SEQ ID NO: 50 |
| | Frame 2 | GGGGGGGGGGGGGGGGWASSARS | |
| | | PPLGGGLPALAGLKRRWRSWWWK | |
| | | CGAPMALSTRHL | |
| | 5′ → 3′ | RRRRCQGACGSAAAAAAAAAAEAA | SEQ ID NO: 51 |
| | Frame 3 | AAAAAAAAGPRAPAAHLSGAGSRR | |
| | 3′ → 5′ | RREPAPERWAAGARGPAAAAAAAA | SEQ ID NO: 52 |
| | Frame 1 | AASAAAAAAAAAALPHAPWQRRLR | |
| | | HRRRPRSP | |
| | 3′ → 5′ | PPPPPPPPPPPPPPPPPPPPRCRTPPGS | SEQ ID NO: 53 |
| | Frame 2 | GASVTAAARARR | |
| | 3′ → 5′ | KAPLEPRTSTTSSSIFSSALLAPGARP | SEQ ID NO: 54 |
| | Frame 3 | REVGCGRSRPSRRRRRRRRRLRRRR | |
| | | RRRRRRAAARPLAAAPPSPPPPALA | |
| SBMA | 5′ → 3′ | VEDSAKLKDGSAVRAGKGLPSAAV | SEQ ID NO: 55 |
| | Frame 1 | QDLPRSFPESVPERARSDPEPGPQAP | |
| | | RGRERSTSRRQFAAAAAAAAAAAA | |
| | | AAAAAAAAAAAAAARD | |
| | 5′ → 3′ | N/A | |
| | Frame 2 | | |
| | 5′ → 3′ | SRTRAPGTQRPRAQHLPAPVCCCCS | SEQ ID NO: 56 |
| | Frame 3 | SSSSSSSSSSSSSSSSSSSSSKRLAPGSS | |
| | | SSSRVRMVLPKPIVEAPQATWSWMR | |
| | | NSNLHSRSRPWSATPREVASQSLEPP | |
| | | WPPARGCRSSCQHLRTRMTQLPHPR | |
| | | CPCWAPLSPA | |
| | 3′ → 5′ | SLAAAAAAAAAAAAAAAAAAAAA | SEQ ID NO: 57 |
| | Frame 1 | AAAAANWRREVLRSRPLGAWGPGS | |
| | | GSLRARSGTDSGKLLGRSWTAAEGR | |
| | | PFPALTALPSLSLAESSTYFPYPASPS | |
| | | LAQKSSTGCDDAVVAAASCPPAGSS | |
| | | REGNLREQPRKKRSDSLKVSCRRRH | |
| | | TVDKICPARLTVCLLILEG | |
| | 3′ 5′ | GLGRTILTLLLLLLPGASLLLLLLLLL | SEQ ID NO: 58 |
| | Frame 2 | LLLLLLLLLLLLLLLQQQQTGAGRC | |
| | | CARGLWVPGARVLDIEFAHALEQILE | |
| | | SSSVGLGRRPRVDPSQP | |
| | 3′ → 5′ | PVGPLRWAWGEPSSPCCCCCCLGLV | SEQ ID NO: 59 |
| | Frame 3 | SCCCCCCCCCCCCCCCCCCCCCCCS | |
| | | SSKLAPGGAALAASGCLGPGFWITS | |
| | | RTLWNRFWKAPR | |
| DRPLA | 5′ → 3′ | N/A | |
| | Frame 1 | | |
| | 5′ → 3′ | SSSSSSSSSSSSSSSITETLGPLLLEHFP | SEQ ID NO: 60 |
| | Frame 2 | THWRAVAPTTHTLTPCLPPWGL | |
| | 5′ → 3′ | AAAAAAAAAAAAAAASRKLWAPSS | SEQ ID NO: 61 |
| | Frame 3 | WSISPPTGGR | |
| | 3′ → 5′ | CCCCCCCCCCCCCCCCCCCWW | SEQ ID NO: 62 |
| | Frame 1 | | |
| | 3′ → 5′ | AAAAAAAAAAAAAAAVAVAGGDG | SEQ ID NO: 63 |
| | Frame 2 | DVLRLVGGRWTGPQ | |
| | 3′ → 5′ | LLLLLLLLLLLLLLLLLLLVVMVMC | SEQ ID NO: 64 |
| | Frame 3 | | |
| SCA1 | 5′ → 3′ | AAAAAAAAAAAAASASAAAAAAAA | SEQ ID NO: 65 |
| | Frame 1 | AAAAAAPQQGSGAHHPGVPPTSPAE | |
| | | PVRPHFQFSAEHRPHRLSSGHPRPPPP | |
| | | PPDDDPTHAHPGAPLPGRHAIRRLRQ | |
| | | PLCPSGGHQES | |
| | 5′ → 3′ | N/A | |
| | Frame 2 | | |
| | 5′ → 3′ | ARRRDTRLSSSSSSSSSSSSSISISSSSS | SEQ ID NO: 66 |
| | Frame 3 | SSSSSSSSSTSAGLRGSSPRGPPHQPS | |
| | | RTSTSTFPVLRRTPAAPPLLRPSPSTS | |
| | | TPTRR | |
| | 3′ → 5′ | CCCCCCCCCCCCCSALCPGVWLRLP | SEQ ID NO: 67 |
| | Frame 1 | MLASRVE | |
| | 3′ → 5′ | GAAAAAAAAAAAAAADADAAAAA | SEQ ID NO: 68 |
| | Frame 2 | AAAAAAAAQPCVPASGSDCPCWPA | |
| | | EWNRPPAGSAGMEWWPLRPRPLHW | |
| | 3′ → 5′ | GRGPVPPALLHLTVQDLLGLDGLLQ | SEQ ID NO: 69 |
| | Frame 3 | PAALSFLGGLPRDKVAAGVGVLHD | |
| | | DLGGGPQGERVWDHRLVGVEVDG | |
| | | DGRRRGGAAGVLRRTGNVDVLVLL | |
| | | GWWGGPRGDEPRSPAEVLLLLLLLL | |
| | | LLLLLLMLMLLLLLLLLLLLLLSLVS | |
| | | RRLAQTAHVGQQSGIGLQLGALGW | |
| | | SGGPCGRGHCTGDGVGGWGDQL | |
| SCA2 | 5′ → 3′ | N/A | |
| | Frame 1 | | |
| | 5′ → 3′ | SPSSSSSSSSSSSSSNSSSSSSSSSRRPR | SEQ ID NO: 70 |
| | Frame 2 | LPMSASPAAAAF | |
| | 5′ → 3′ | QRQRRRRVSARLPAAPWSRRASPPL | SEQ ID NO: 71 |
| | Frame 3 | RRPPSPPRQPGRPSGRANPRLPARRP | |
| | | RVPAAFRRLLGAPGSRLSPPGVRAG | |
| | | VWAPHHVAEAPAAAAAAAAAAAA | |
| | | ATAAAAAAAAAAARGCQCPQARRQ | |
| | | RPSSVARRRAFAVLVLGLLVLGHGS | |
| | | LLGGRGDLRRREARPGQRSKQ | |
| | 3′ → 5′ | KAAAAGLADIGSRGRRLLLLLLLLL | SEQ ID NO: 72 |
| | Frame 1 | LLLLLLLLLLLLLLGLQRHGEGPIHR | |
| | | LARRAGTAGSRARQGDAGTRRGRA | |
| | | GAERGGAGWRGRRGARAGEGEKE | |
| | | DDEGAGRPAETKEPPGAGPKRAAA | |
| | | VAVATKTV | |
| | 3′ → 5′ | GSPLLLFRPLPRPGLPPPEVAATTEEG | SEQ ID NO: 73 |
| | Frame 2 | AVAEDEETEDEDGEGAAAGDARRP | |
| | | LPPGLRTLAAAGGGCCCCCCCCCCC | |
| | | CCCCCCCCCCCWGFSDMVRGPYTG | |
| | | SHAGRGQPGAGRAKETPERGGDAR | |
| | | APSGEARVGAAGGAPGLARGRRRT | |
| | | TKGRGGPPRPRSRREPGRNAPPPLPL | |
| | | LPKQSEAEGGELCREGGGPGPGGGG | |
| | | AAEGYGPGAAPPPPRPLRRAGRWSE | |
| | | RHPGHLAAAKRRDSVATAGLRGAA | |
| | | AAERIGGRARRGAGWERRCG | |
| | 3′ → 5′ | TEAVLCYCFDLCPGRASRRRRSPRPP | SEQ ID NO: 74 |
| | Frame 3 | RREPWPRTRRPRTRTAKARRRATLE | |
| | | GRCRRACGHWQPRAAAAAAAAAA | |
| | | AVAAAAAAAAAAAAAGASATW | |
| SCA3 | 5′ → 3′ | N/A | |
| | Frame 1 | | |
| | 5′ → 3′ | HRHQVQILLQKSFGRDEKPTLKNSS | SEQ ID NO: 75 |
| | Frame 2 | KSSNSSSSSSSRGTYQDRVHIHVKGQ | |
| | | PPVQEHLGVI | |
| | 5′ → 3′ | KTAAKAATAAAAAAAGGPIRTEFTS | SEQ ID NO: 76 |
| | Frame 3 | M | |
| | 3′ → 5′ | VPLLLLLLLLLLLLLLFFKVGFSSLPK | SEQ ID NO: 77 |
| | Frame 1 | LF | |
| | 3′ → 5′ | CCCCCCCCCCFCCCFSK | SEQ ID NO: 78 |
| | Frame 2 | | |
| | 3′ → 5′ | AAAAAAAVAAFAAVFQSRLLVSSE | SEQ ID NO: 79 |
| | Frame 3 | ALLK | |
| SCA6 | 5′ → 3′ | SVRPAARGPRSSSSSSSSSSSSRRWPG | SEQ ID NO: 80 |
| | Frame 1 | RAGRPPAALGGTQAPRPSLWPEIGRP | |
| | | RGATAAAARPGWRGGSQARPGASP | |
| | | PGPVDTAGPGGRHLARTCPRGPRVP | |
| | | GTMATTGAPTTTRPMARAAGAARR | |
| | | PWPGPTTRHPPYDTRPRAPPGARPG | |
| | | LPGPRARPAPRLLGTAGDSPTATTRR | |
| | | TDWPGPAGRAPGRACTNPTARVTMI | |
| | | GAKPGRGGARPAPHAPHAHTPPEEP | |
| | | RRGRGGPAQRARERASRETPDSGEA | |
| | | RAGPQGCPAETLGQKRPSWAATAPP | |
| | | NQPRSPHPRQGLSGGROGADKPHSQ | |
| | | GI | |
| | 5′ → 3′ | GRRLGAPAAAAAAAAAAAAAGGG | SEQ ID NO: 81 |
| | Frame 2 | QAGPGGHQRPSEVPRPHGRASGRRS | |
| | | AAHGGPQQRPLAQDGEAGPRPGPER | |
| | | VPQGLSTRRGPVAGIWPARVRGAPG | |
| | | SPAPWLLPGLRLRRGRWPGQRGRR | |
| | | GGHGRGLRRATPRTTRVLGRHRAL | |
| | | AQDSPGLGPGLRLAFSARPATPQRLL | |
| | | PGARTGOAPRAGLQEGPARTLQRE | |
| | 5′ → 3′ | N/A | |
| | Frame 3 | | |
| | 3′ → 5′ | PPAAAAAAAAAAAAAGAPSRRPYG | SEQ ID NO: 82 |
| | Frame 1 | SQGNRTRVAGGWRGSGGAGGGPAA | |
| | | ECWYQMLRGLGFHLRNYCPAGAPC | |
| | | ALGPRWASGTSAGPEPVPGRGPAVP | |
| | | GHSGPCRGAGDGGGGGGGGGAVD | |
| | | ASDPWAGPAPGPAPSTAGPRIGWSC | |
| | | SGLSPSLCPHRCSGPGSAQRRGGCG | |
| | | RGAAGGAAGSPRAGPAPASNRPGV | |
| | | GPWGPARRLNASWGWCLRWY | |
| | 3′ → 5′ | LLLLLLLLLLLLLRGPRAAGLTDHRG | SEQ ID NO: 83 |
| | Frame 2 | IGHVWPGGGGGLGELAAAPPRSAGT | |
| | | RC | |
| | 3′ → 5′ | CCCCCCCCCCCCCGGPEPPALRITGE | SEQ ID NO: 84 |
| | Frame 3 | | |
| SCA7 | 5′ → 3′ | AAAAAAAAAAAAASAAPAAAAPAT | SEQ ID NO: 85 |
| | Frame 1 | AATAHTAGGRRARRRLHLGRRNGD | |
| | | GRGAQASAQS | |
| | 5′ → 3′ | N/A | |
| | Frame 2 | | |
| | 5′ → 3′ | SSSSSSSSSSRRLRSPSGSSTRHRRHG | SEQ ID NO: 86 |
| | Frame 3 | AHGRRTAGPAPPPPRPPQWRRSGSA | |
| | | GLCPVLK | |
| | 3′ → 5′ | GGGGGCCCRWGCGGGGCCCCCCC | SEQ ID NO: 87 |
| | Frame 1 | CCCRAAAAAAPPAAAAARRGSPLTS | |
| | | SAARSDILSAPFLWRVGQKS | |
| | 3′ → 5′ | NFRPILSRPSQEVWKPQPTDSTTVPA | SEQ ID NO: 88 |
| | Frame 2 | SLQDWAEACAPRPSPLRRPRWRRRR | |
| | | ARRPPAVCAVAAVAGAAAAGAAEA | |
| | | AAAAAAAAAAAAGRPRPLLRPPPPP | |
| | | RGAAPP | |
| | 3′ → 5′ | LLLLLLLLLLPGGRGRCSARRRRRA | SEQ ID NO: 89 |
| | Frame 3 | ARLPPDVIRGPLRHSFRSFSLEGRPKI | |
| | | LINLPMDLHLLQL | |
| SCA17 | 5′ → 3′ | N/A | |
| | Frame 1 | | |
| | 5′ → 3′ | PHSLFRTPIVCLFWKSNKGSSSNNNS | SEQ ID NO: 90 |
| | Frame 2 | SSSSSSSNSNSSSSSSSSSSSSSSSSSSS | |
| | | NRQWQLQPFSSQRPSRQHREPQARH | |
| | | HSSSTHRLSQLHPCRAPLHCIPPP | |
| | 5′ → 3′ | SVYFGRATKAAAATTTAAAAAAAA | SEQ ID NO: 91 |
| | Frame 3 | TATAAAAAAAAAAAAAAAAAAAT | |
| | | GSGSCSRSAVNVPAGNTGNLRPGTT | |
| | | ALPLTDSHNCTLAGHHSTVSLPHDS | |
| | | HDPHHSCHASFGEFVVDCTAAAKYCI | |
| | | HSESWL | |
| | 3′ → 5′ | LLNGCSCHCLLLLLLLLLLLLLLLLL | SEQ ID NO: 92 |
| | Frame 1 | LLLLLLLLLLLLLLLLLLLLLPLLLFQ | |
| | | NRQTIGVLNRLWGQSSAIRHHWTKD | |
| | | RDSGSHGTLRGGOALSVRWQAVVLI | |
| | | HDVHFLLGKPETLALELVSLFNFFLE | |
| | | HLQHTLLSNFLNSLGYLHTPRNSDA | |
| | | GSLQRSLWASGSEVKQPAAQAPATA | |
| | | NLPDLTEPLARVDNVTSA | |
| | 3′ → 5′ | TAAAATACCCCCCCCCCCCCCCCC | SEQ ID NO: 93 |
| | Frame 2 | CCCCCCCCCCCCCCCCCCCCCLCCS | |
| | | SKIDRLLVF | |
| | 3′ → 5′ | ESVSGRAVVPGLRFPVLPAGTLTAE | SEQ ID NO: 94 |
| | Frame 3 | RLQLPLPVAAAAAAAAAAAAAAAA | |
| | | AAAVAVAAAAAAAAVVVAAAAFV | |
| | | ALPK | |
| CTG18.1 | 5′ → 3′ | NPNRLPSGALSCCCCCCCCCCCCCC | SEQ ID NO: 95 |
| | Frame 1 | CCCCCCCCCCCSSSSSSFSSSSSSSRP | |
| | | SFGEMAFGSFARKRSPRQAALQPPF | |
| | | CLLHFLHSFLCFLQALTQGRCALSTR | |
| | | YVEEEGNQLGSK | |
| | 5′ → 3′ | KESTKHTNKIQTAFQVGLFHAAAAA | SEQ ID NO: 96 |
| | Frame 2 | AAAAAAAAAAAAAAAAAAAAAPPP | |
| | | PPSPPPPPLLDLLLEKWLSEVLPGNV | |
| | | ALGRQLCSPLSACCTFSIRSFAFCRL | |
| | 5′ → 3′ | N/A | |
| | Frame 3 | | |
| | 3′ → 5′ | KRRRRRRRRRRRRSSSSSSSSSSSSSS | SEQ ID NO: 97 |
| | | SSSSSSSSSSSMKEPHLEGGLDFICVF | |
| | Frame 1 | CGFFLFCFTNASYTKLIWH | |
| | 3′ → 5′ | N/A | |
| | Frame 2 | | |
| | 3′ → 5′ | N/A | |
| | Frame 3 | | |

Portions of amino acid sequences depicted in Table 1 with single underlining identify C-terminal sequences; portions of amino acid sequences depicted in Table 1 with double underlining identify N-terminal sequences. N/A indicates reading frames in which translation is ATG-initiated.

An antibody composition that specifically binds to at least a portion of a polypeptide described herein can permit one to identify whether a candidate polypeptide is a polypeptide of the invention. Thus, in some embodiments, a composition can include a polypeptide that specifically binds to an antibody composition that specifically binds to at least a portion of a polypeptide known to be a RAN-translated polypeptide such as, for example, an antibody composition that specifically binds to at least a portion of a polypeptide shown in Table 1.

An antibody composition of the invention can include one or more antibodies prepared in any suitable manner such as, for example, one or more monoclonal antibodies, a polyclonal antibody preparation, or one or more antibodies that are produced recombinantly. Antibody compositions including monoclonal antibodies and/or anti-idiotypes can also be prepared using known methods. Chimeric antibodies include human-derived constant regions of both heavy and light chains and murine-derived variable regions that are antigen-specific (Morrison et al., Proc. Natl. Acad. Sci. USA, 1984, 81(21):6851-5; LoBuglio et al., Proc. Natl. Acad. Sci. USA, 1989, 86(11):4220-4; Boulianne et al., Nature, 1984, 312(5995):643-6.). Humanized antibodies substitute the murine constant and framework (FR) (of the variable region) with the human counterparts (Jones et al., Nature, 1986, 321(6069):522-5; Riechmann et al., Nature, 1988, 332(6162):323-7; Verhoeyen et al., Science, 1988, 239(4847):1534-6; Queen et al., Proc. Natl. Acad. Sci. USA, 1989, 86(24):10029-33; Daugherty et al., Nucleic Acids Res., 1991, 19(9): 2471-6.). Alternatively, certain mouse strains can be used that have been genetically engineered to produce antibodies that are almost completely of human origin; following immunization the B cells of these mice are harvested and immortalized for the production of human monoclonal antibodies (Bruggeman and Taussig, Curr. Opin. Biotechnol., 1997, 8(4):455-8; Lonberg and Huszar, Int. Rev. Immunol., 1995; 13(1):65-93; Lonberg et al., Nature, 1994, 368:856-9; Taylor et al., Nucleic Acids Res., 1992, 20:6287-95.). A polyclonal antibody composition may be isolated from any suitable source such as, for example, serum, plasma, blood, colostrum, and the like.

In another aspect, the invention provides a method for detecting expression of a polypeptide described herein. These methods may be useful for detecting whether a subject is expressing polypeptides expressed from nucleotide expansions associated with certain conditions. Generally, the method includes receiving a biological sample from a subject, detecting whether the biological sample comprises a RAN-translated polypeptide associated with a condition characterized at least in part by a nucleotide repeat expansion and identifying the subject as at risk for a condition characterized by a repeat expansion if the biological sample includes the RAN-translated polypeptide. In some cases, the RAN-translated polypeptide may be detected by combining at least a portion of the sample with antibody that specifically binds to at least a portion of a RAN-translated polypeptide such as, for example, antibody as described immediately above. However, a RAN-translated polypeptide may be detected by any suitable protein detection method known to those skilled in the art such as, for example, any chromatography, spectrometry, electrophoresis, and the like.

A subject identified as expressing a polypeptide as described herein may be considered “at risk” for developing such a condition even if, at the time of the identification, the subject does not exhibit any symptoms or clinical signs of the condition.

Thus, for example, referring to Table 1, detecting expression of SEQ ID NO:20, SEQ ID NO:21, SEQ ID NO:22, SEQ ID NO:23, SEQ ID NO:24, or SEQ ID NO:25 can identify a subject as having or as being at risk of developing Type 1 myotonic dystrophy (DM1). One exemplary way of detecting expression of SEQ ID NO:20, SEQ ID NO:21, SEQ ID NO:22, SEQ ID NO:23, SEQ ID NO:24, or SEQ ID NO:25 can include contacting at least a portion of the biological sample with an antibody composition that specifically binds to at least a portion of at least one of SEQ ID NO:20, SEQ ID NO:21, SEQ ID NO:22, SEQ ID NO:23, SEQ ID NO:24, or SEQ ID NO:25 and determining whether the antibody composition specifically binds to a component—i.e., a RAN-translated polypeptide—in the biological sample.

As another example, detecting expression of SEQ ID NO:26, SEQ ID NO:27, SEQ ID NO:28, SEQ ID NO:29, SEQ ID NO:30, or SEQ ID NO:31 can identify a subject as having or as being at risk of developing Type 2 myotonic dystrophy (DM2). One exemplary way of detecting expression of SEQ ID NO:26, SEQ ID NO:27, SEQ ID NO:28, SEQ ID NO:29, SEQ ID NO:30, or SEQ ID NO:31 can include contacting at least a portion of the biological sample with an antibody composition that specifically binds to at least a portion of at least one of SEQ ID NO:26, SEQ ID NO:27, SEQ ID NO:28, SEQ ID NO:29, SEQ ID NO:30, or SEQ ID NO:31 and determining whether the antibody composition specifically binds to a component—i.e., a RAN-translated polypeptide—in the biological sample.

As another example, detecting expression of SEQ ID NO:32, SEQ ID NO:33, SEQ ID NO:34, SEQ ID NO:35, or SEQ ID NO:36 can identify a subject as having or as being at risk of developing Huntington's Disease (HD). One exemplary way of detecting expression of SEQ ID NO:32, SEQ ID NO:33, SEQ ID NO:34, SEQ ID NO:35, or SEQ ID NO:36 can include contacting at least a portion of the biological sample with an antibody composition that specifically binds to at least a portion of at least one of SEQ ID NO:32, SEQ ID NO:33, SEQ ID NO:34, SEQ ID NO:35, or SEQ ID NO:36 and determining whether the antibody composition specifically binds to a component—i.e., a RAN-translated polypeptide—in the biological sample.

As another example, detecting expression of SEQ ID NO:37, SEQ ID NO:38, SEQ ID NO:39, SEQ ID NO:40, SEQ ID NO:41, or SEQ ID NO:42 can identify a subject as having or as being at risk of developing Huntington's Disease-like 2 (HDL2). One exemplary way of detecting expression of SEQ ID NO:37, SEQ ID NO:38, SEQ ID NO:39, SEQ ID NO:40, SEQ ID NO:41, or SEQ ID NO:42 can include contacting at least a portion of the biological sample with an antibody composition that specifically binds to at least a portion of at least one of SEQ ID NO:37, SEQ ID NO:38, SEQ ID NO:39, SEQ ID NO:40, SEQ ID NO:41, or SEQ ID NO:42 and determining whether the antibody composition specifically binds to a component—i.e., a RAN-translated polypeptide—in the biological sample.

As another example, detecting expression of SEQ ID NO:49, SEQ ID NO:50, SEQ ID NO:51, SEQ ID NO:52, SEQ ID NO:53, or SEQ ID NO:54 can identify a subject as having or as being at risk of developing a Fragile X-associated condition such as, for example, Fragile X Syndrome (FRAXA or FRAXE) or Fragile X Tremor/Ataxia Syndrome (FXTAS). One exemplary way of detecting expression of SEQ ID NO:49, SEQ ID NO:50, SEQ ID NO:51, SEQ ID NO:52, SEQ ID NO:53, or SEQ ID NO:54 can include contacting at least a portion of the biological sample with an antibody composition that specifically binds to at least a portion of at least one of SEQ ID NO:49, SEQ ID NO:50, SEQ ID NO:51, SEQ ID NO:52, SEQ ID NO:53, or SEQ ID NO:54 and determining whether the antibody composition specifically binds to a component—i.e., a RAN-translated polypeptide—in the biological sample.

As another example, detecting expression of SEQ ID NO:55, SEQ ID NO:56, SEQ ID NO:57, SEQ ID NO:58, or SEQ ID NO:59 can identify a subject as having or as being at risk of developing Spinal Bulbar Muscular Atrophy (SMBA). One exemplary way of detecting expression of SEQ ID NO:55, SEQ ID NO:56, SEQ ID NO:57, SEQ ID NO:58, or SEQ ID NO:59 can include contacting at least a portion of the biological sample with an antibody composition that specifically binds to at least a portion of at least one of SEQ ID NO:55, SEQ ID NO:56, SEQ ID NO:57, SEQ ID NO:58, or SEQ ID NO:59 and determining whether the antibody composition specifically binds to a component—i.e., a RAN-translated polypeptide—in the biological sample.

As another example, detecting expression of SEQ ID NO:60, SEQ ID NO:61, SEQ ID NO:62, SEQ ID NO:63, or SEQ ID NO:64 can identify a subject as having or as being at risk of developing Dentatorubropallidoluysian Atrophy (DRPLA). One exemplary way of detecting expression of SEQ ID NO:60, SEQ ID NO:61, SEQ ID NO:62, SEQ ID NO:63, or SEQ ID NO:64 can include contacting at least a portion of the biological sample with an antibody composition that specifically binds to at least a portion of at least one of SEQ ID NO:60, SEQ ID NO:61, SEQ ID NO:62, SEQ ID NO:63, or SEQ ID NO:64 and determining whether the antibody composition specifically binds to a component—i.e., a RAN-translated polypeptide—in the biological sample.

As another example, detecting expression of SEQ ID NO:65, SEQ ID NO:66, SEQ ID NO:67, SEQ ID NO:68, or SEQ ID NO:69 can identify a subject as having or as being at risk of developing Spinocerebellar Ataxia 1 (SCA1). One exemplary way of detecting expression of SEQ ID NO:65, SEQ ID NO:66, SEQ ID NO:67, SEQ ID NO:68, or SEQ ID NO:69 can include contacting at least a portion of the biological sample with an antibody composition that specifically binds to at least a portion of at least one of SEQ ID NO:65, SEQ ID NO:66, SEQ ID NO:67, SEQ ID NO:68, or SEQ ID NO:69 and determining whether the antibody composition specifically binds to a component—i.e., a RAN-translated polypeptide—in the biological sample.

As another example, detecting expression of SEQ ID NO:70, SEQ ID NO:71, SEQ ID NO:72, SEQ ID NO:73, or SEQ ID NO:74 can identify a subject as having or as being at risk of developing Spinocerebellar Ataxia 2 (SCA2). One exemplary way of detecting expression of SEQ ID NO:70, SEQ ID NO:71, SEQ ID NO:72, SEQ ID NO:73, or SEQ ID NO:74 can include contacting at least a portion of the biological sample with an antibody composition that specifically binds to at least a portion of at least one of SEQ ID NO:70, SEQ ID NO:71, SEQ ID NO:72, SEQ ID NO:73, or SEQ ID NO:74 and determining whether the antibody composition specifically binds to a component—i.e., a RAN-translated polypeptide—in the biological sample.

As another example, detecting expression of SEQ ID NO:75, SEQ ID NO:76, SEQ ID NO:77, SEQ ID NO:78, or SEQ ID NO:79 can identify a subject as having or as being at risk of developing Spinocerebellar Ataxia 3 (SCA3). One exemplary way of detecting expression of SEQ ID NO:75, SEQ ID NO:76, SEQ ID NO:77, SEQ ID NO:78, or SEQ ID NO:79 can include contacting at least a portion of the biological sample with an antibody composition that specifically binds to at least a portion of at least one of SEQ ID NO:75, SEQ ID NO:76, SEQ ID NO:77, SEQ ID NO:78, or SEQ ID NO:79 and determining whether the antibody composition specifically binds to a component—i.e., a RAN-translated polypeptide—in the biological sample.

As another example, detecting expression of SEQ ID NO:80, SEQ ID NO:81, SEQ ID NO:82, SEQ ID NO:83, or SEQ ID NO:84 can identify a subject as having or as being at risk of developing Spinocerebellar Ataxia 6 (SCA6). One exemplary way of detecting expression of SEQ ID NO:80, SEQ ID NO:81, SEQ ID NO:82, SEQ ID NO:83, or SEQ ID NO:84 can include contacting at least a portion of the biological sample with an antibody composition that specifically binds to at least a portion of at least one of SEQ ID NO:80, SEQ ID NO:81, SEQ ID NO:82, SEQ ID NO:83, or SEQ ID NO:84 and determining whether the antibody composition specifically binds to a component—i.e., a RAN-translated polypeptide—in the biological sample.

As another example, detecting expression of SEQ ID NO:85, SEQ ID NO:86, SEQ NO:87, SEQ ID NO:88, or SEQ ID NO:89 can identify a subject as having or as being at risk of developing Spinocerebellar Ataxia 7 (SCA7). One exemplary way of detecting expression of SEQ ID NO:85, SEQ ID NO:86, SEQ ID NO:87, SEQ ID NO:88, or SEQ ID NO:89 can include contacting at least a portion of the biological sample with an antibody composition that specifically binds to at least a portion of at least one of SEQ ID NO:85, SEQ ID NO:86, SEQ ID NO:87, SEQ ID NO:88, or SEQ ID NO:89 and determining whether the antibody composition specifically binds to a component—i.e., a RAN-translated polypeptide—in the biological sample.

As another example, detecting expression of SEQ ID NO:14, SEQ ID NO:15, SEQ ID NO:16, SEQ ID NO:17, SEQ ID NO:18, SEQ ID or NO:19 can identify a subject as having or as being at risk of developing Spinocerebellar Ataxia 8 (SCA8). One exemplary way of detecting expression of SEQ ID NO:14, SEQ ID NO:15, SEQ ID NO:16, SEQ ID NO:17, SEQ ID NO:18, SEQ ID or NO:19 can include contacting at least a portion of the biological sample with an antibody composition that specifically binds to at least a portion of at least one of SEQ ID NO:14, SEQ ID NO:15, SEQ ID NO:16, SEQ ID NO:17, SEQ ID NO:18, SEQ ID or NO:19 and determining whether the antibody composition specifically binds to a component—i.e., a RAN-translated polypeptide—in the biological sample.

As another example, detecting expression of SEQ ID NO:43, SEQ ID NO:44, SEQ ID NO:45, SEQ ID NO:46, SEQ ID NO:47, SEQ ID or NO:48 can identify a subject as having or as being at risk of developing Spinocerebellar Ataxia 12 (SCA12). One exemplary way of detecting expression of SEQ ID NO:43, SEQ ID NO:44, SEQ ID NO:45, SEQ ID NO:46, SEQ ID NO:47, SEQ ID or NO:48 can include contacting at least a portion of the biological sample with an antibody composition that specifically binds to at least a portion of at least one of SEQ ID NO:43, SEQ ID NO:44, SEQ ID NO:45, SEQ ID NO:46, SEQ ID NO:47, SEQ ID or NO:48 and determining whether the antibody composition specifically binds to a component—i.e., a RAN-translated polypeptide—in the biological sample.

As another example, detecting expression of SEQ ID NO:90, SEQ ID NO:91, SEQ ID NO:92, SEQ ID NO:93, or SEQ ID NO:94 can identify a subject as having or as being at risk of developing Spinocerebellar Ataxia 17 (SCA17). One exemplary way of detecting expression of SEQ ID NO:90, SEQ ID NO:91, SEQ ID NO:92, SEQ ID NO:93, or SEQ ID NO:94 can include contacting at least a portion of the biological sample with an antibody composition that specifically binds to at least a portion of at least one of SEQ ID NO:90, SEQ ID NO:91, SEQ ID NO:92, SEQ ID NO:93, or SEQ ID NO:94 and determining whether the antibody composition specifically binds to a component—i.e., a RAN-translated polypeptide—in the biological sample.

As yet another example, detecting expression of SEQ ID NO:95, SEQ ID NO:96, or SEQ ID NO:97 can identify a subject as having or as being at risk of developing a condition characterized, at least in part, by a repeat expansion at the CTG18.1 locus. One exemplary way of detecting expression of SEQ ID NO:95, SEQ ID NO:96, or SEQ ID NO:97 can include contacting at least a portion of the biological sample with an antibody composition that specifically binds to at least a portion of at least one of SEQ ID NO:95, SEQ ID NO:96, or SEQ ID NO:97 and determining whether the antibody composition specifically binds to a component—i.e., a RAN-translated polypeptide—in the biological sample.

Thus, in certain embodiments, the method includes contacting an antibody composition that specifically binds to a polypeptide described herein with a biological sample obtained from the subject. In such embodiments, the method further includes incubating the mixture under conditions to allow the antibody to specifically bind the polypeptide to form a polypeptide:antibody complex. As used herein, the term “polypeptide:antibody complex” refers to the complex that results when an antibody specifically binds to a polypeptide. The biological sample and/or the antibody composition may include one or more reagents such as, for example, a buffer, that provide conditions appropriate for the formation of the polypeptide:antibody complex. The polypeptide:antibody complex is then detected. The detection of antibodies is known in the art and can include, for instance, immunofluorescence or peroxidase. The methods for detecting the presence of antibodies that specifically bind to polypeptides of the present invention can be used in various formats that have been used to detect antibody, including radioimmunoassay and enzyme-linked immunosorbent assay.

In another aspect, RAN-translated polypeptides can serve as biomarkers for certain conditions associated with nucleotide repeat expansions. Certain methods provided herein exploit RAN-translated polypeptides as biomarkers for such conditions.

In one method, detecting biomarkers expressed from nucleotide expansions associated with certain conditions can provide information regarding the efficacy of treatment of such a condition. Similar methods are known using ATG-initiated biomarkers associated with, for example, HD and HDL2. Generally, certain therapeutic methods involve administering to a subject an inhibitory therapeutic oligonucleotide (e.g., siRNA) to inhibit translation of mRNA transcripts that encode biomarkers known to be associated with a particular condition. Thus, for example, detecting a biomarker expressed from a nucleotide expansion associated with the condition (by, for example, using antibody that specifically binds to the biomarker) can provide temporal information regarding the efficacy of administering the antisense therapeutic oligonucleotide. For example, a biomarker can be detected prior to the commencement of therapy, detected again after a specified period of therapy, and any difference in the amount of the biomarker can be determined, thereby evaluating efficacy of the therapy.

In another method, detecting biomarkers expressed from nucleotide expansions associated with certain conditions can help identify specific tissues in a subject in which a biomarker is expressed. Generally, samples can be obtained from a plurality of tissues of a subject. Each sample may be analyzed (by, for example, using antibody that specifically binds to the biomarker) to determine whether differential expression of the biomarker exists in the subject. For example, polypeptide biomarkers associated with HD and/or HDL2 may be found in blood, heart, muscle, and/or brain tissue.

The present invention exploits the discovery that in the absence of an ATG codon, expanded nucleotide repeats may be translated. This unexpected Repeat Associated Non-ATG translation or RAN-translation occurs in mammalian tissue culture, rabbit-reticulocyte lysates, and lentiviral vector transduced mouse brains. RAN-translation results in the production of novel polypeptides encoded by otherwise noncoding nucleotide sequences. This RAN-translation occurs in a variety of disease-relevant sequence contexts suggesting that this phenomenon may occur in a wide range of repeat diseases. For example, CAG and CTG trinucleotide repeats such as those associated with, for example, spinocerebellar ataxia type 8 (SCA8), often express homopolymeric expansion proteins in all three frames: polyQ, polyA, and/or polyS for CAG expansions and polyL, polyA, and/or polyC for CUG expansions. Finally, antibodies specific for two putative non-ATG initiated proteins provide strong in vivo evidence that the predicted SCA8_{GCA-Ala} and DM1_{CAG-Gln} expansion proteins are expressed in disease relevant tissues. In SCA8, specific staining for the SCA8_{GCA-Ala} expansion protein is found in cerebellar Purkinje cells and in DM1, staining for the DM1_{CAG-Gln} expansion protein is found in cardiac myocytes, skeletal muscle, and leukocytes.

Our understanding of the molecular basis of human disease has been built on studying the expected effects that disease mutations have on their corresponding genes. For microsatellite-expansion disorders the position of mutations has been used to broadly group repeat expansions located in predicted coding and non-coding regions into protein loss-, protein gain-, or RNA gain-, of-function categories. Cell culture and animal models have in turn been developed to test specific hypotheses under the assumption that (a) CAG expansion mutations located in polyQ ORFs only express protein in the ATG-initiated polyQ frame, and (b) expansions located in non-coding regions do not encode proteins. We have found the expression of additional novel and unexpected poly-amino acid expansion proteins expressed in the absence of ATG initiation.

While initiation at specific alternative codons has been previously reported, our findings are novel with respect to the flexibility with which translation initiation occurs at CAG.CTG expansion sites. Our results show that RAN-translation of CAG expansions occurs in a wide variety of sequence contexts, including in the presence of upstream sequences from the HD, HDL2, SCA3, SCA8 and DM1 loci. Additionally we show RAN-translation depends on repeat length, with CAG repeats of about 42, but not 15, sufficient for non-ATG translation of polyQ protein and longer tracks of 70-100 repeats needed for polyA and polyS expression.

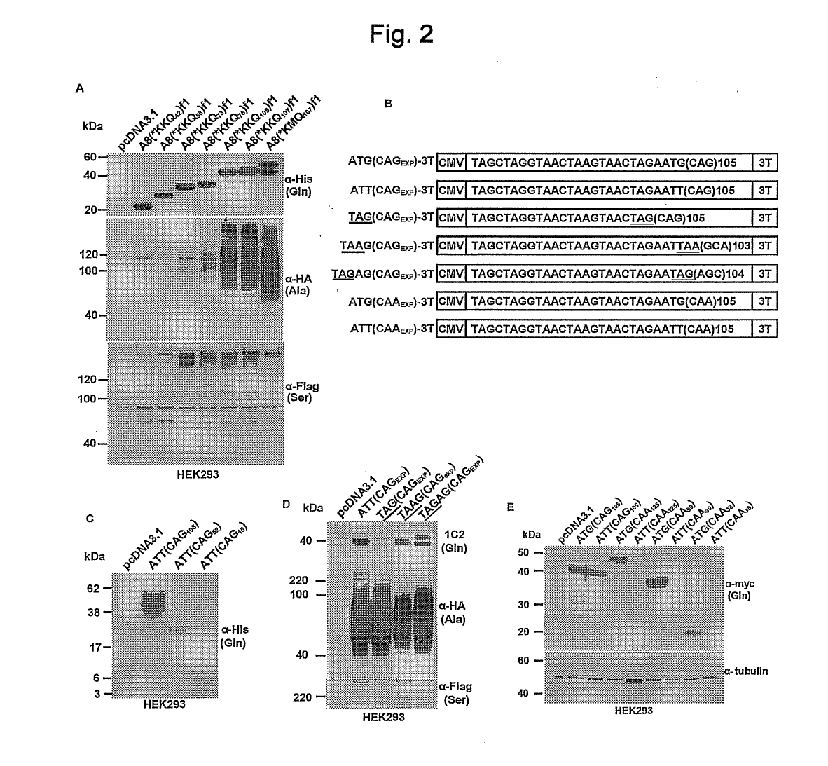

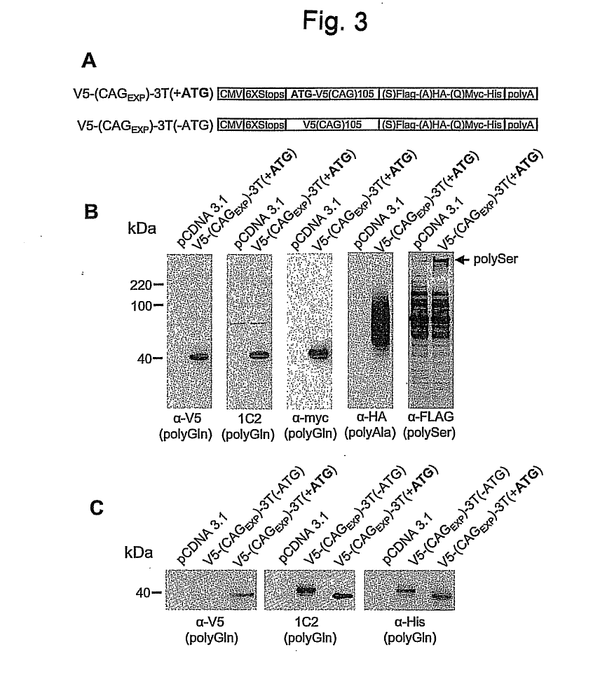

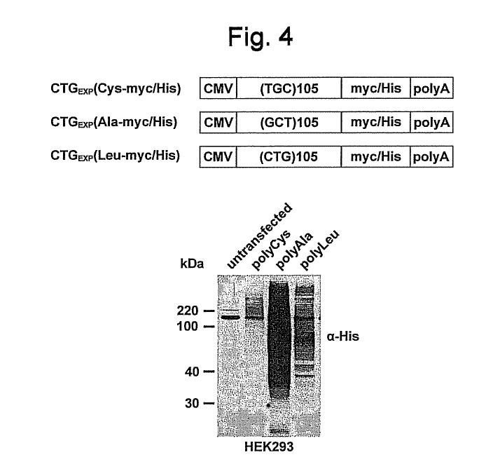

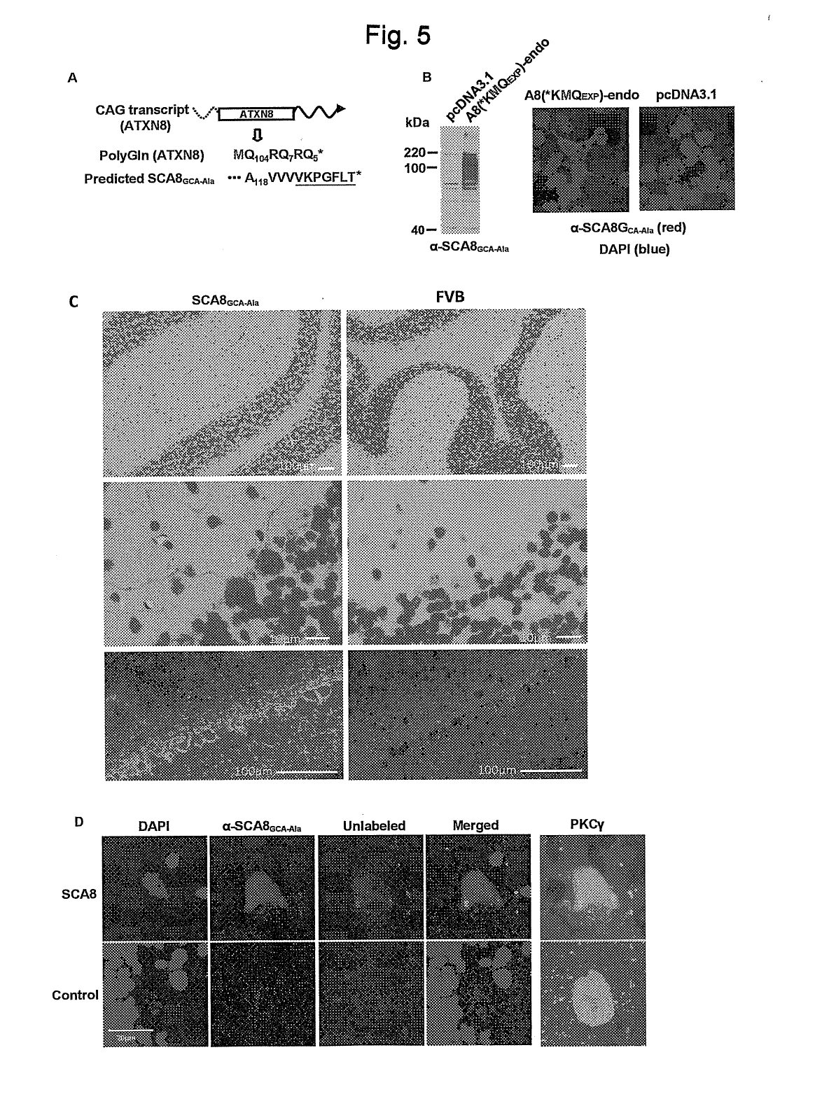

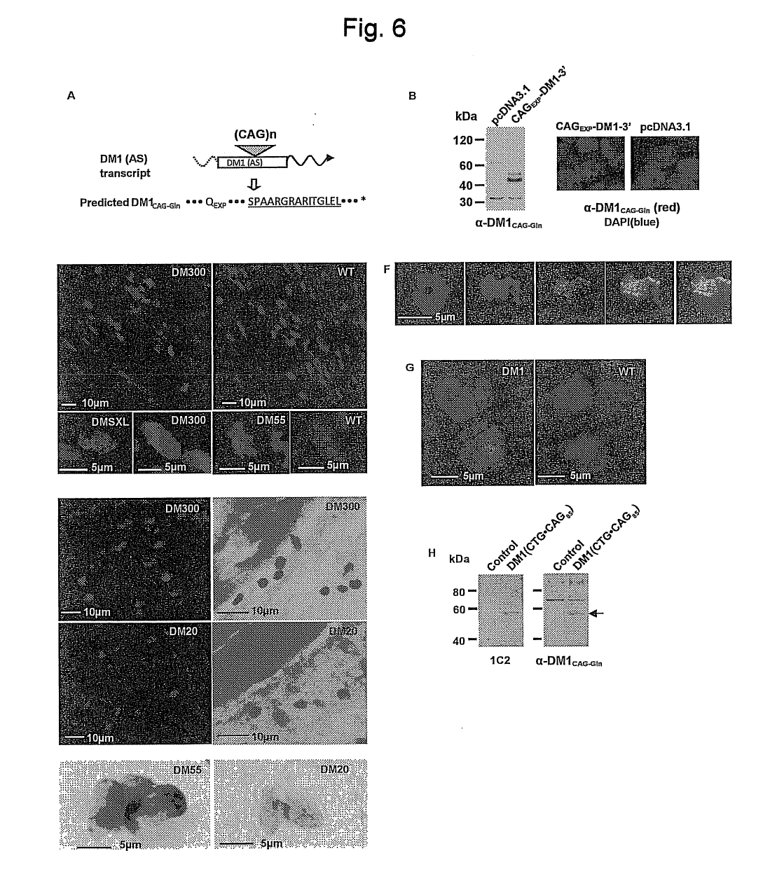

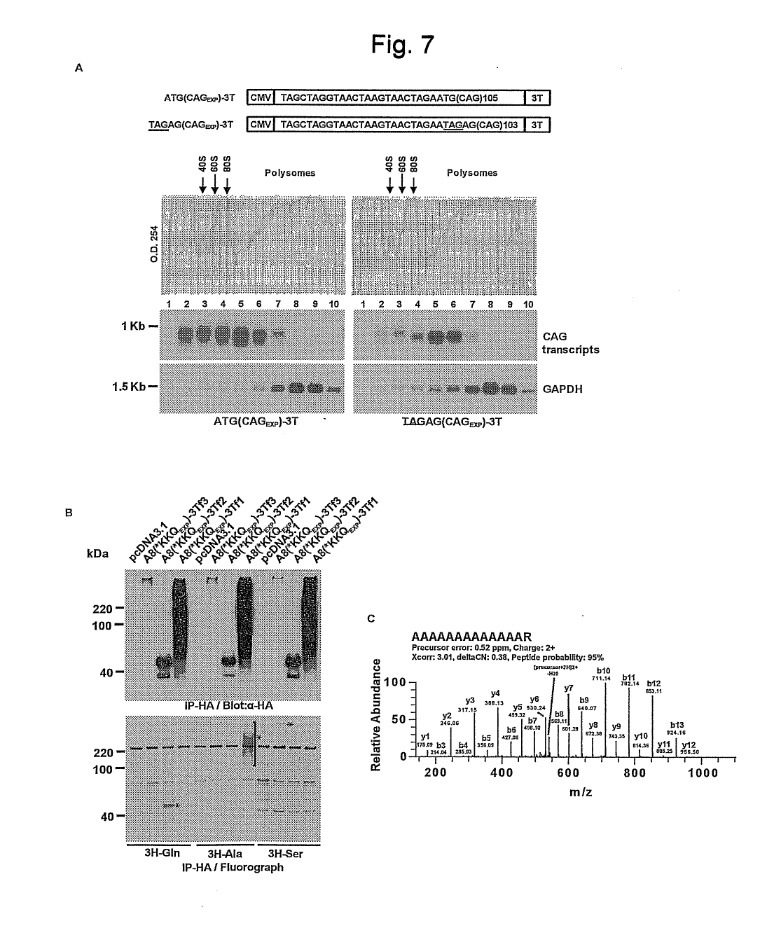

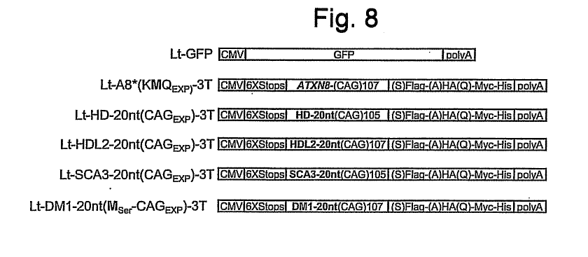

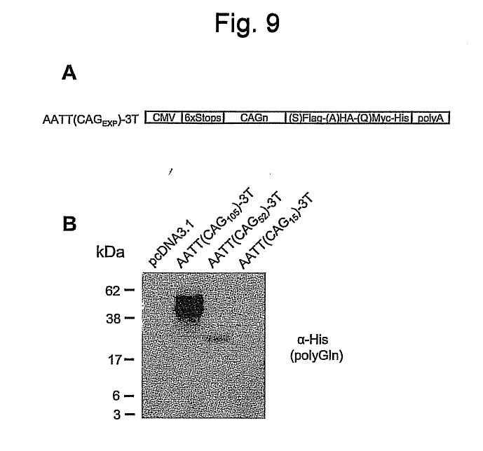

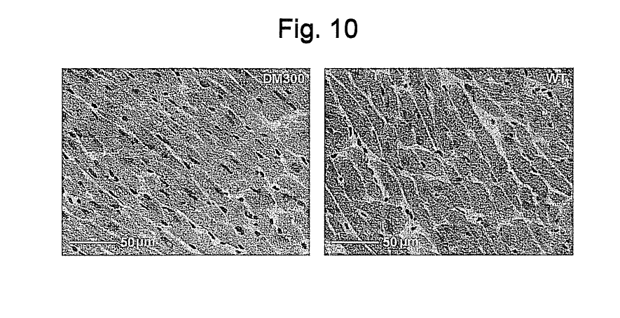

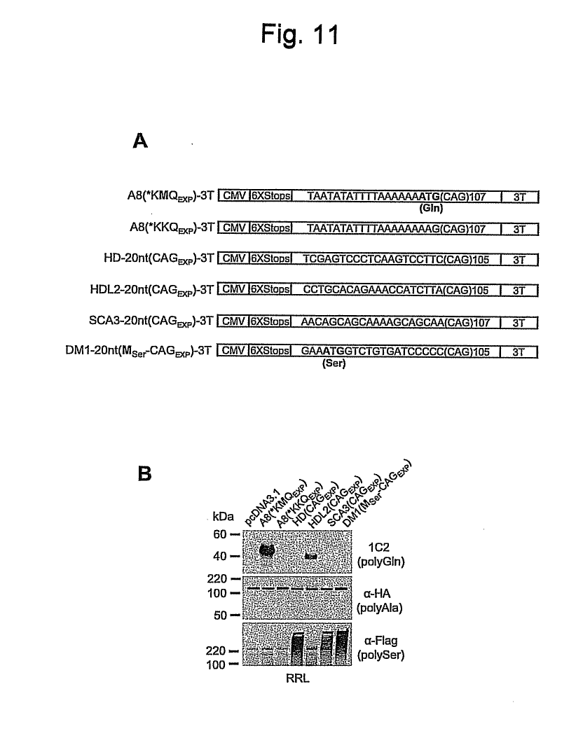

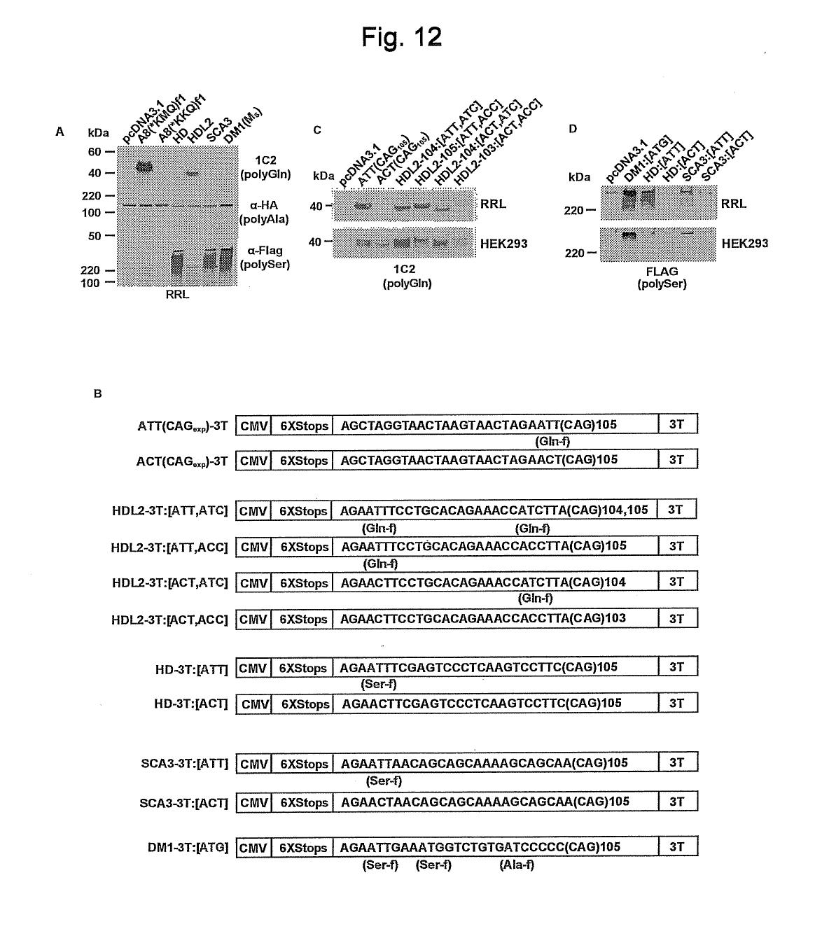

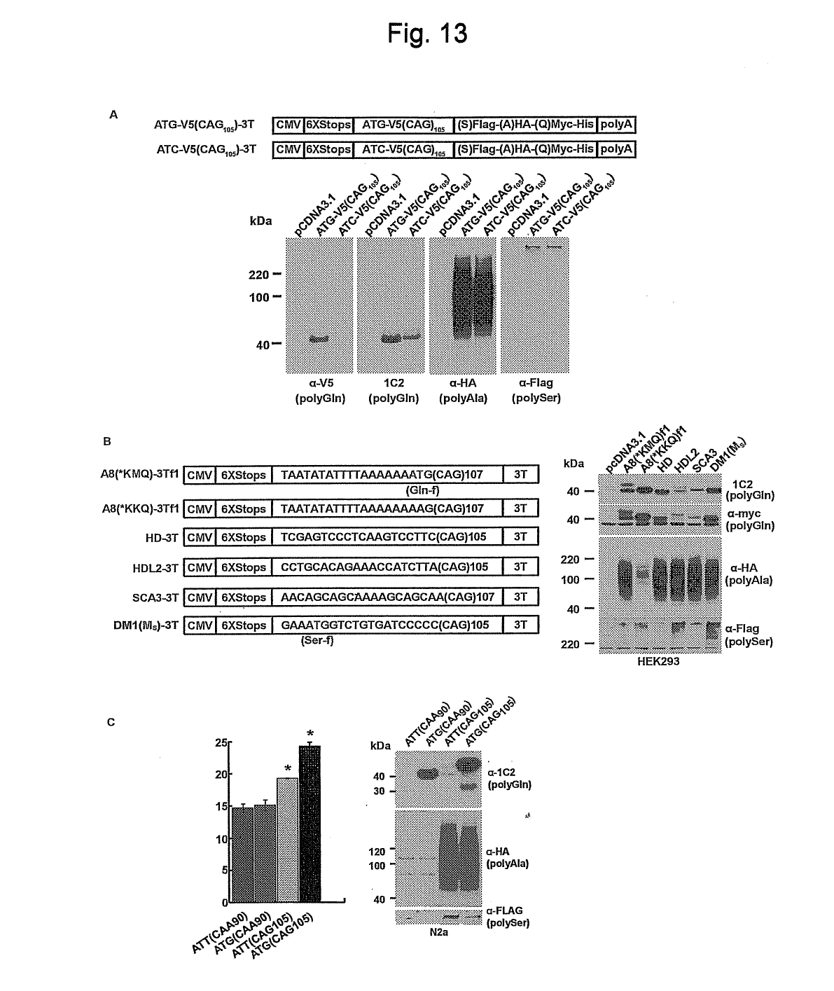

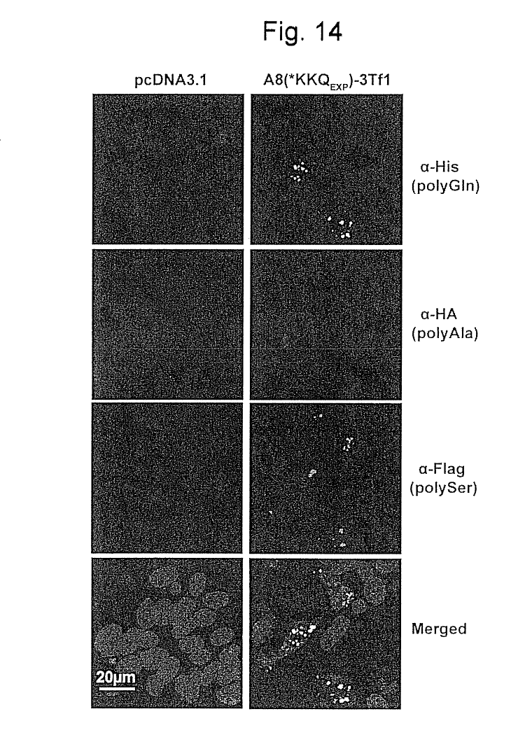

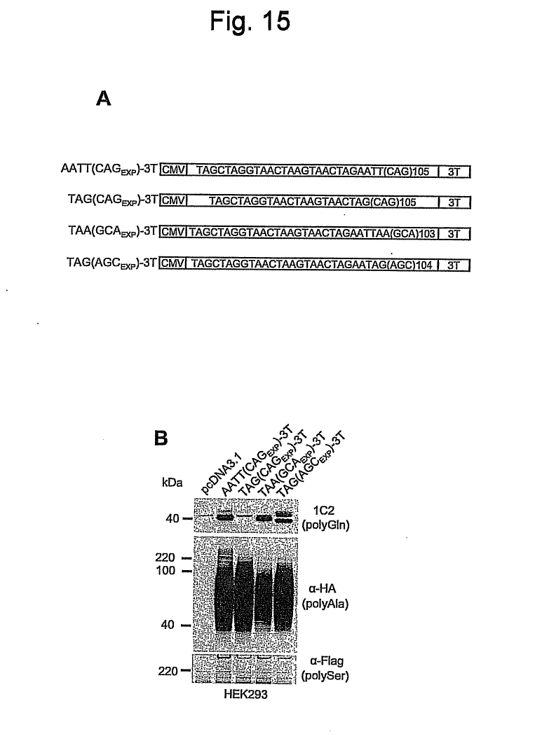

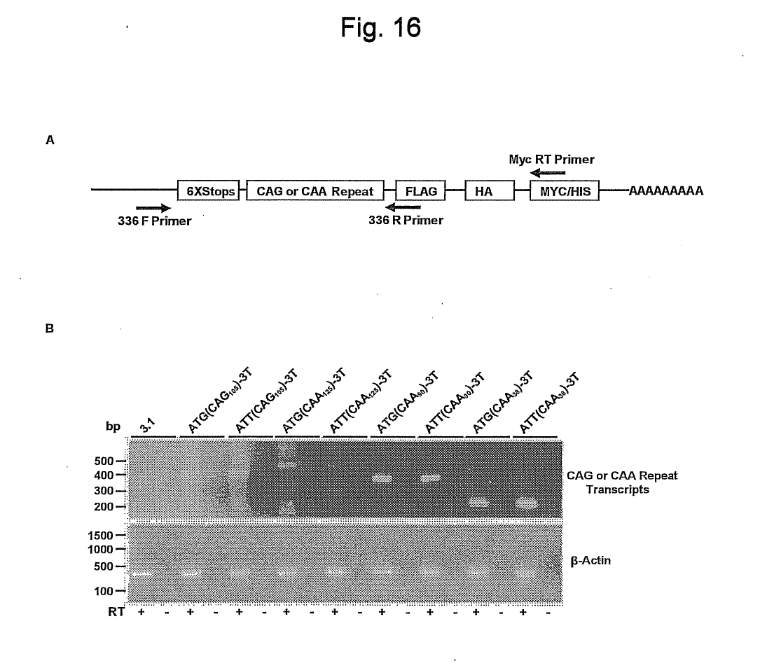

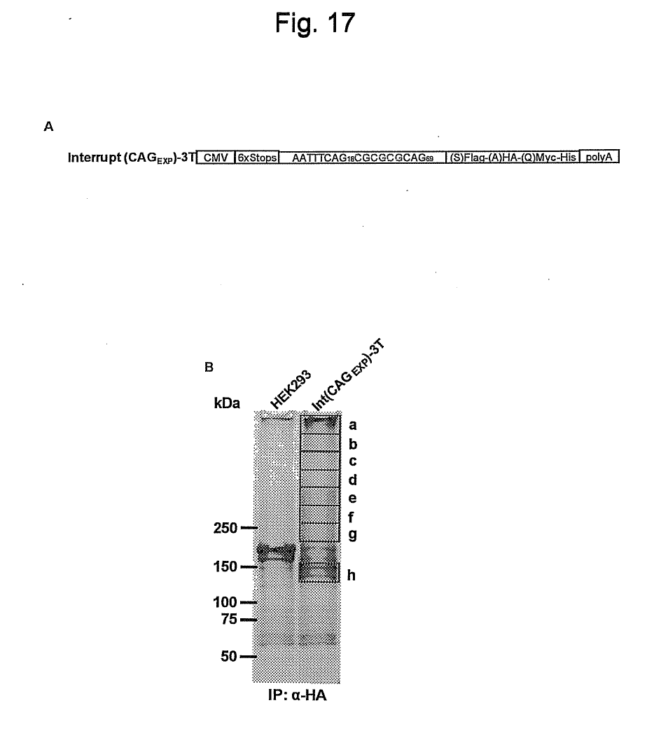

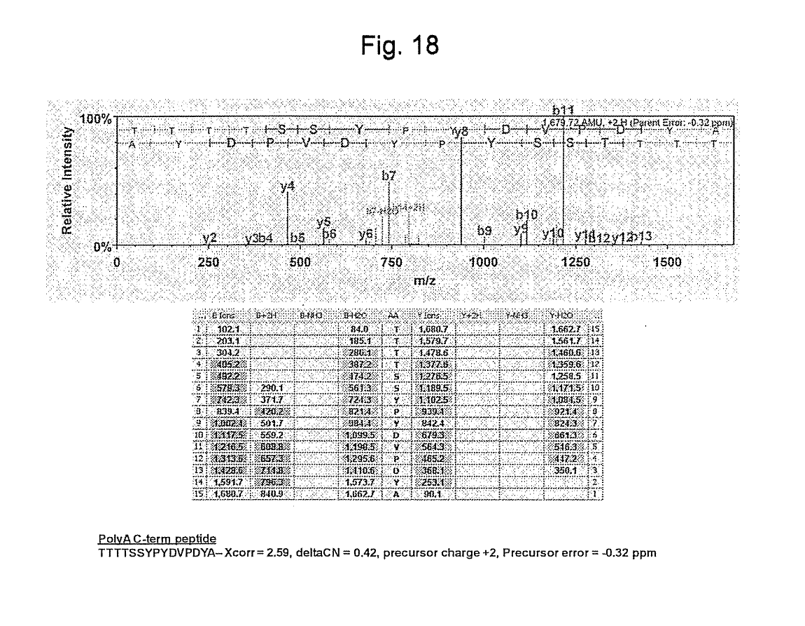

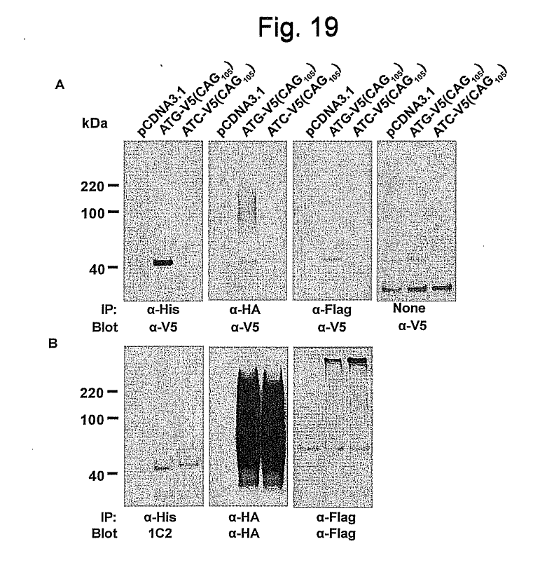

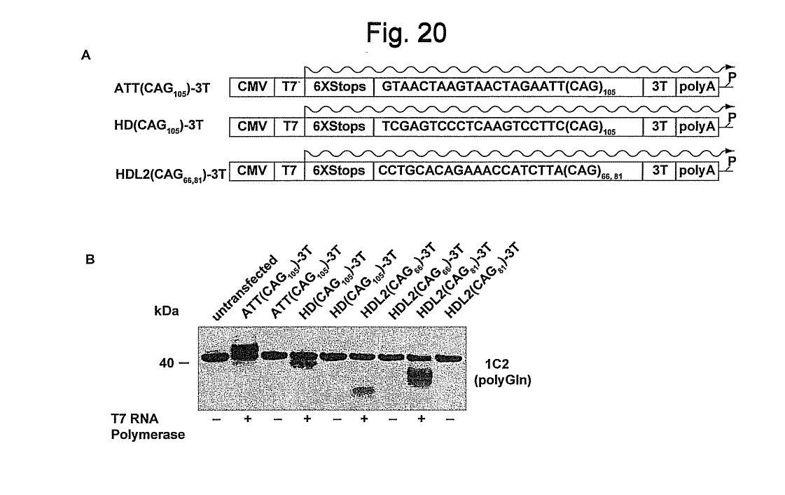

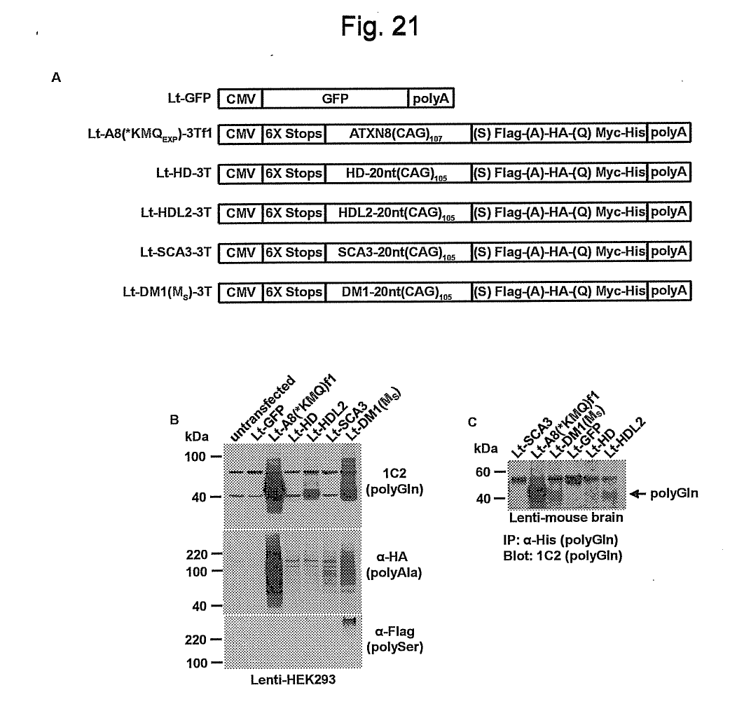

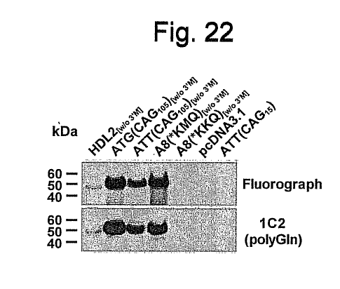

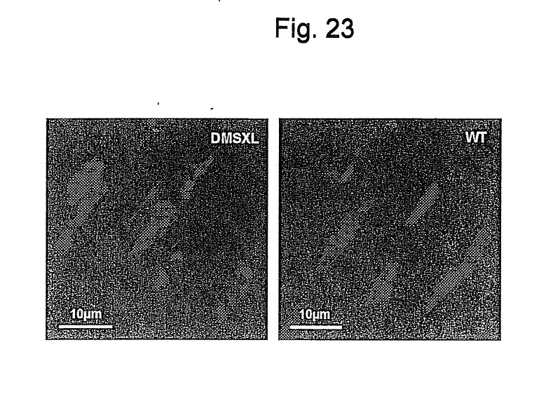

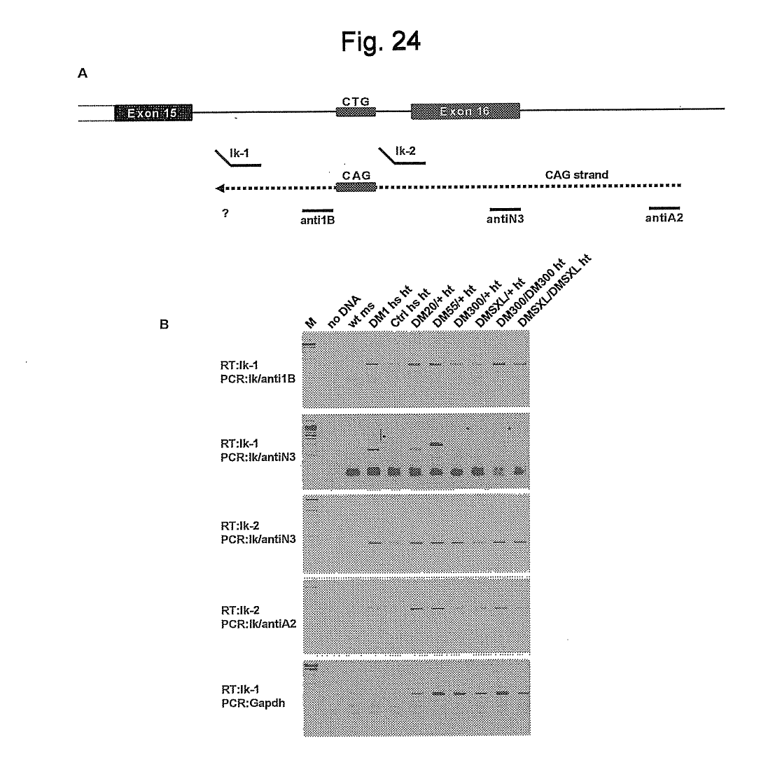

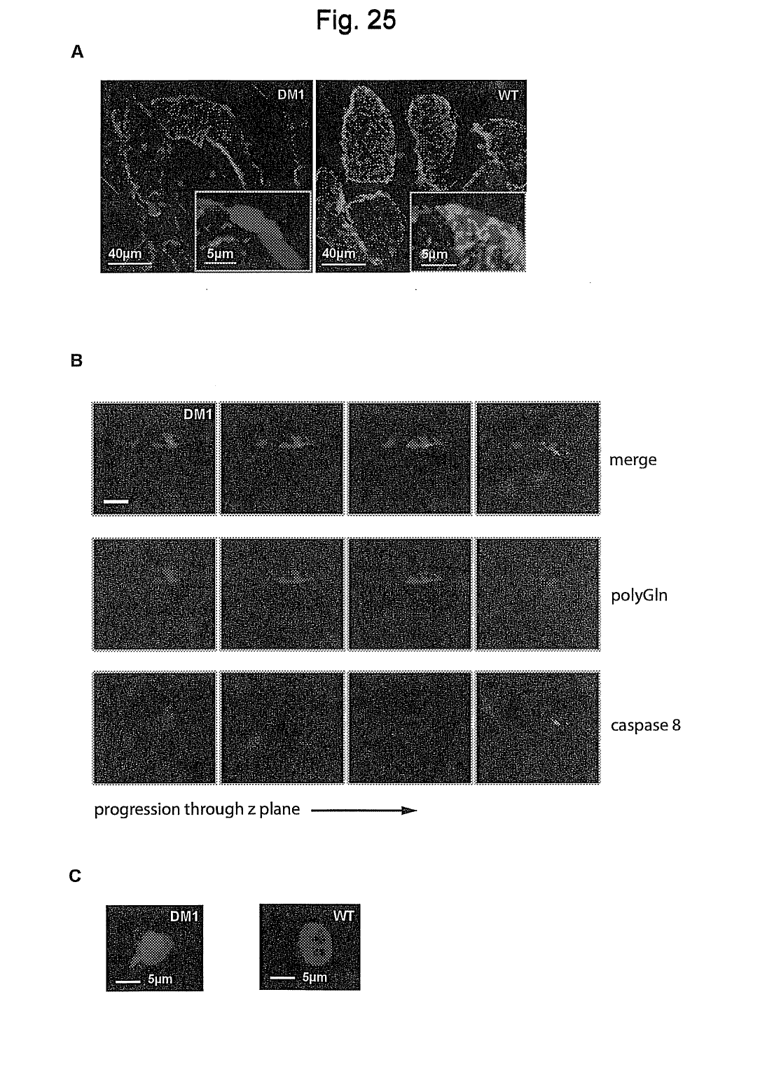

Several observations we have made provide mechanistic insights into RAN-translation. First, epitope-tag experiments show non-ATG translation of the polyQ tract can be initiated at one or a few specific sites close to or within the repeat tract (FIG. 3C). Second, RAN-translation of polyA and polyS can occur when the CAG expansion is located within or outside of an ATG-initiated polyQ ORF (FIG. 3B), suggesting that disease-causing CAG expansions in polyQ ORFs may also express polyA and polyS and that expansions located in previously described “non-coding” regions may express homopolymeric proteins in all frames. Third, repeat motifs that form hairpin structures (CAG and CUG) show robust RAN-translation compared to non-hairpin forming CAA expansions. Hairpin sequences have previously been shown to facilitate translation initiation at non-ATG codons and it is possible that they play a similar role in RAN-translation at expansion disorder loci. Fourth, two separate experimental modifications selectively inhibit the expression of one or more homopolymeric proteins while permitting robust expression of another. The insertion of a TAG-stop codon immediately preceding the CAG_{EXP} [TAG(CAG_{EXP})-3T] inhibits translation of polyQ but not polyA (FIG. 2A). Additionally, in vitro translation in rabbit-reticulocyte lysates prevents the translation of polyA, while allowing the HDL2, HD, and SCA3 constructs to express a single homopolymeric protein (FIG. 11B). These results indicate that upstream sequence and cellular factors influence RAN-translation and that individual reading frames can be differentially affected.

Mass spectrometry of polyA expansion protein detected by epitope tags confirms that the polyA protein migrates as a high molecular weight smear by PAGE and that translational initiation does not require an ATG initiation codon. Because translational initiation in eukaryotes normally requires a met-tRNAⁱ and methionine incorporation, we searched for but found no evidence for any peptides in which a methionine codon is incorporated. In contrast, we identified a series of peptide fragments that begin with and contain various numbers of alanine. These results suggest that translation initiation either occurs without incorporating an N-terminal methionine or that if an N-terminal methionine is incorporated it is rapidly removed by methionine aminopeptidase or endopeptidase activity. According to the N-end rule, both N-terminal Ala and Ser residues would serve as stabilizing residues that could cause these proteins to accumulate in the cell. In contrast to the RAN-translation which occurs in cells, the non-ATG translation found in the RRLs is limited and has more stringent sequence requirements consistent with those previously described by others involving only a single mismatch nucleotide change from the canonical AUG start codon (ATT and ATC) (FIG. 12).

Additionally, our data show that the expression of the polyA and polyS proteins can occur without frameshifting out of an ATG initiated polyQ frame. Although frameshifting has been previously suggested to result in the expression of hybrid polyQ-polyA and polyQ-polyS proteins in SCA3 and HD, our results (FIG. 3) suggest RAN-translation, rather than frameshifting, can account for the expression of pure polyA and polyS.

Expression of homopolymeric proteins from CAG.CTG expansions can occur via one or more possible mechanisms. First, one or more types of RNA editing (ADAR, CDAR or insertional) could cause sequence changes within or upstream of the repeat in a subpopulation of transcripts. RNA editing of specific genes has been reported in humans, but the idea that CAG and CUG transcripts could direct abundant posttranscriptional modifications in a wide variety of sequence contexts is novel. A second possible mechanism is that proximal CAG and CUG hairpins perturb the normal translation process and allow the use of previously undocumented alternative initiation sites.

Our observations support involvement of polyA and polyS expansion proteins in some of the CAG-polyQ diseases and that homopolymeric proteins contribute to diseases thought to primarily involve RNA gain-of-function effects (e.g., Type 1 myotonic dystrophy, DM1). Substantial evidence from model systems demonstrate that nearly all of these homopolymeric expansion proteins are toxic: polyQ, polyA, polyS, polyC, and polyL. Additionally we show that RAN translation increases apoptotic cell death in N2a cells (FIG. 13D). For many of the adult onset polyQ disorders (e.g., SBMA, SCA1, SCA2, etc.), patients tend to have shorter expansions that would be less likely to show RAN-translation. In contrast, the more severe juvenile-onset cases of these disorders, as well as diseases in which expansions are typically longer (e.g., SCA3, SCA8, DM1), may be more likely to express homopolymeric expansion proteins by RAN-translation. Our studies suggest sequence context, repeat length and cell type (FIGS. 2, 6 and 13) play a role in whether or not RAN-translation will lead to the expression of polyQ, polyA and/or polyS proteins. For example, RAN-translation is more likely to occur when expansions are >70 repeats (FIG. 13) and that expression of homopolymeric polyA and polyS proteins may contribute to the repeat length-dependent anticipation seen diseases previously categorized as polyQ disorders.

An additional layer of complexity is that a growing number of expansion disorders involve bidirectional expression (e.g., DM1, SCA7, SCA8, and/or FMR1). While most of the work on polyQ disorders has involved investigations of the protein encoded by the CAG expansion transcript, the DM1 field has focused on the CUG expansion RNAs. While there is clear and compelling evidence that RNA gain-of-function effects mediated by CUG (e.g., DM1) or CCUG (e.g., Type 2 myotonic dystrophy, DM2) expansion transcripts cause a spliceopathy and many of the clinical parallels between these disorders, our discovery of a DM1 polyQ protein may explain the more severe disease often found in DM1 vs. DM2 patients. Although polyGln positive cells in DM1 heart, skeletal muscle and myoblast cultures are relatively rare, the DM1-polyGln protein is readily detectable in blood. Further studies are needed to understand the relative contributions of these toxic proteins in disease.

Additionally, our discovery that RAN-translation of the CAG expansion transcript leads to the accumulation of the novel DM1 polyQ-expansion protein, DM1_{CAG-Gln} highlights the need to investigate the potential pathogenic effects of both expansion transcripts. Given that CAG and CUG expansion transcripts can express homopolymeric proteins without an ATG, and that CUG, and more recently CAG expansion transcripts have been reported to cause RNA gain-of-function effects, it is possible that the molecular pathology of these disorders will turn out to be far more complex than we initially appreciated, with the potential expression of up to six toxic expansion proteins and two toxic expansion RNAs. Our data suggest that future therapies that focus on reducing expression of these expansion transcripts or the size of the expansion itself are likely to be the most efficacious.

Non-ATG Translation of Homopolymeric polyQ, polyA, and polyS Expansion Proteins.

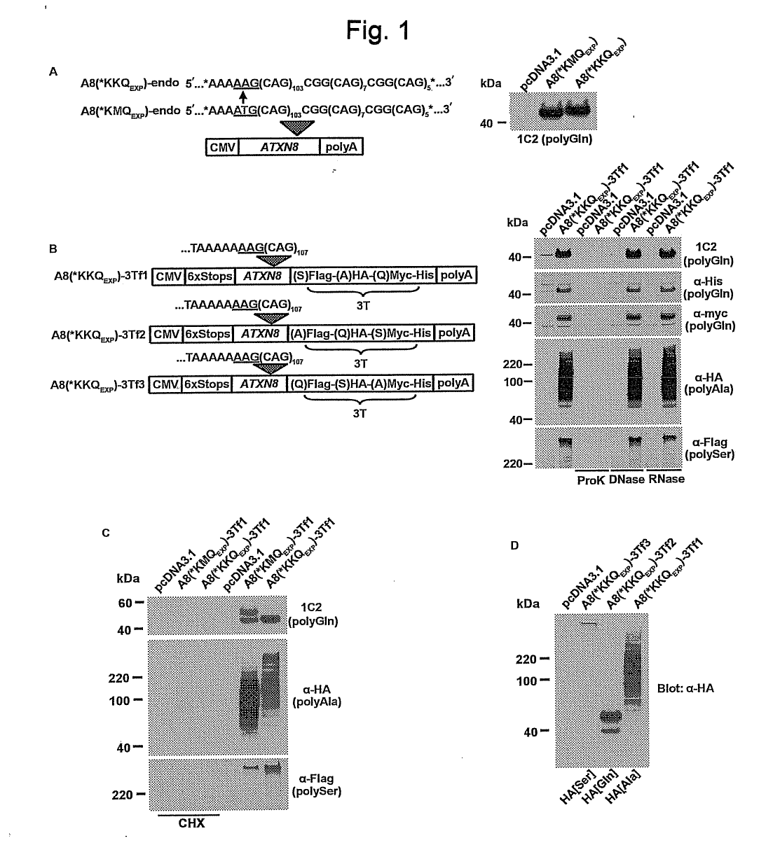

To understand the role of the ATXN8 polyQ protein in SCA8, we mutated the only ATG initiation codon located 5′ of the CAG expansion on an ATXN8 (A8) minigene and unexpectedly found that this mutation did not prevent expression of the polyQ-expansion protein in transfected HEK293 cells (FIG. 1A). Sequence analysis showed neither full-length nor spliced transcripts, which are expressed at approximately equal ratios from this minigene, are predicted to contain an AUG initiation codon. To test if non-ATG translation could also occur in other frames, a triply-tagged A8 minigene, A8(*KKQ_{EXP})-3Tf1, was generated by inserting a 6× STOP codon cassette (two stops in each frame) upstream of the CAG_{EXP} and three different C-terminal tags to monitor protein expression in all three reading frames (i.e., CAG glutamine [Gln]; AGC, serine [Ser]; GCA alanine [Ala]) (FIG. 1B). Surprisingly, although the corresponding transcripts were confirmed to lack initiator AUG codons, tagged polyQ, polyA, and polyS proteins were expressed (FIG. 1B) in transfected HEK293 cells.

The polyQ expansion protein migrated as bands of one or more discrete molecular weights suggesting that translation initiation occurs at specific sites and not randomly throughout the repeat. In contrast, the polyA protein migrated as a robust high-molecular weight smear and the polyS protein showed a third migration pattern near the top of the gel when separated by polyacrylamide gel electrophoresis (PAGE) in SDS (FIG. 1B) or 8M urea (not shown). As expected these proteins are degraded by proteinase K, but not RNase I or DNase I (FIG. 1B) and are not made in the presence of cycloheximide (FIG. 1C). As seen in FIG. 1C, the presence of an ATG start codon in the polyQ frame can result in the generation of a second, higher molecular weight band and this sequence change also affects the migration pattern of the polyA protein and the relative levels of the polyS protein.

A direct comparison of the relative levels of these proteins, each expressed with an HA tag, shows that the polyQ and polyA are present at relatively high levels with lower levels of polyS (FIG. 1D). Immunofluorescence staining of cells transfected with the triply-tagged A8(*KKQEXP)-3Tf1 construct shows that polyQ, polyA and polyS proteins can be simultaneously expressed in a single cell and that the relative levels of these proteins in transfected cells can vary dramatically (FIG. 14).

### RAN-Translation Depends on Repeat Length.

To test the effects of repeat length on this repeat-associated non-ATG or RAN-translation, A8(*KKQEXP)-3Tf1 constructs containing 42-107 CAGs were transfected into HEK293 cells and detected by immunoblot. PolyQ proteins were detected in cells transfected with all repeat lengths (FIG. 2A). Additionally, polyQ protein was detected in cells transfected with the ATT(CAG_{EXP})-3T construct containing 105 and 52, but not 15 repeats (FIG. 2B). PolyA proteins were most robustly expressed from constructs containing longer repeats (107 and 105), moderately expressed with 78 and 73 repeats, and no longer detectable with 58 and 42 repeats (FIG. 2A). PolyS protein was detected in cells transfected with constructs containing 58-107 repeats but not 42 repeats (FIG. 2A). These data demonstrate that non-ATG initiation of all three homopolymers is length-dependent and that RAN-translation of polyA and polyS proteins requires longer repeat tracts than polyQ.

### RAN-Translation in Presence of Immediate Upstream Stop Codons.

To test the effects of sequence context on RAN-translation, we modified the A8(*KKQEXP)-3Tf1 construct by removing 90 bp of ATXN8 sequence so the 6×-STOP cassette is almost adjacent to the CAG_{EXP} and placing an additional seventh TAG- or TAA-stop codon immediately upstream of polyQ, polyA, and polyS frames (FIG. 2C). These constructs, which express full-length unspliced transcripts in transfected HEK293 cells (data not shown), also express polyQ and polyA but only low levels of polyS with the exception that the construct containing the TAG-stop immediately preceeding the glutamine frame prevents translation of polyQ but not the polyA or polyS proteins (FIG. 2C).

RAN-Translation from Hairpin-Forming CAG and CTG but not CAA Repeats.

Next we tested the effects of the repeat motif on RAN-translation by comparing the expression of the polyQ-expansion proteins expressed from constructs containing hairpin-forming CAG and non-hairpin forming CAA repeats. Cells transfected with CAG expansion constructs with or without ATG start codons express polyQ proteins (FIG. 2D). In contrast, polyQ protein is only expressed from the CAA expansion constructs in the presence of an ATG start codon, strongly suggesting that hairpin formation plays a role in RAN-translation. All constructs were confirmed to express repeat containing transcripts by RT-PCR (FIG. 16).

Because CUG transcripts faun hairpin structures and because the SCAB and DM1 expansion mutations are bidirectionally expressed, we tested if RAN-translation can also occur in the CTG direction. Similar to the CAG expansions, cells transfected with CTG expansion constructs with no upstream ATGs in any frame robustly express homopolymeric-proteins in all three frames, polyL, polyA and polyC (FIG. 4).

Non-AUG Containing Transcripts Co-Migrate with Light Polyribosomal Fractions.

To characterize the repeat containing transcripts and to better understand the mechanism of RAN translation we purified mRNA from actively translating polyribosomes isolated from HEK293 cells transfected with (CAG_{EXP})-3T constructs with and without an ATG initiation codon (FIG. 7A). Northern analysis shows that transcripts expressed from both the +ATG and −ATG constructs co-sediment with the light polyribosomal fractions. Additionally a large fraction of the ATG(CAGexp)-3T mRNA also co-sediments with untranslated mRNP. (FIG. 7A). The highest levels of CAG_{EXP} transcripts for the −ATG constructs are found in the light polysomal fractions. 5′ RACE and RT-PCR of ribosome bound CAG transcripts show: 1) the predicted transcription start site is used; 2) the sequence predicted by the DNA is found in the corresponding transcripts; 3) no upstream AUG initiation codons have been introduced by RNA editing.

[³H] Labeling of Homopolymeric polyQ, polyA, and polyS Proteins.

To independently demonstrate that these homopolymeric proteins contain polyQ, polyA and polyS tracts we preformed a [³H] labeling experiment. HEK293 cells transfected with triple-tagged constructs containing the HA-tag in the Ala [A8(*KKQ_{EXP})-3Tf1], Gln [A8(*KKQ_{EXP})-3Tf2], or Ser [A8(*KKQ_{EXP})-3Tf3] frames were grown in the presence of [³H]-Gln, [³H]-Ala, or [³H]-Ser amino acids. Proteins were immunoprecipitated using α-HA antibody, separated by PAGE on duplicate gels and detected by either immunoblot or fluorography. The protein blot (FIG. 7B, upper panel) shows that all three proteins in each set are pulled down by IP. The corresponding fluorograph (FIG. 7B, bottom panel) shows [³H]-Gln is preferentially incorporated into the ˜40 kDa protein with the HA-tag in the polyQ frame. Similarly, [³H]-Ala, and [³H]-Ser are preferentially incorporated into proteins immunoprecipitated with tags in the polyA and polyS reading frames, respectively.

Mass Spectrometry Identifies Acetylated and Unacetylyated polyA Peptides of Varying Lengths.

We used mass spectrometry as an additional independent method to confirm the identity of this unexpected non-ATG translation. We selected the polyA protein for this analysis because a polyA antibody is not available and because this putative polyA protein is expressed at sufficiently high levels required for mass spectrometry. HEK293 cells were transfected using a modified CAG expansion construct in which a 5′ 6×-STOP cassette was inserted almost adjacent to the CAG_{EXP} with an HA tag located at the 3′ end of the repeat in the polyA frame (FIG. 17A). Additionally, we modified the repeat tract by inserting an arginine codon after 18 GCA alanine codons so that trypsin digestion of the N-terminal portion of protein would generate fragments of suitable size for mass spectrometry (FIG. 17A). Associated mass spectra were submitted for database searching against a human protein database plus a list of all possible polyA proteins in which translation could occur before or within the repeat tract and which initiation would allow for the possible inclusion of an N-terminal methionine residue. We identified a series of N-terminally acetylated and un-acetylated peptides containing varying numbers of alanines: [(A)₈₋₁₈R], IS(A)₁₈R and S(A)₁₈R (FIG. 7C and FIG. 17). No peptides containing an N-terminal methionine residue were identified. Additionally, the predicted C-terminal digestion fragment (TTTTSSYPYDVPDYA, SEQ ID NO:134) of the polyA protein was identified (FIG. 18). These results demonstrate that RAN translation across the (CAG) expansion results in the expression of polyA expansion proteins in transfected HEK293 cells and that these proteins are co- or post-translationally modified. Additionally, the identification of peptides of with varying numbers of alanines from regions a-h of the preparative gel confirms that the polyA expansion proteins run as a broad smear when separated by SDS-PAGE (FIG. 17).

RAN-Translation of polyA and polyS Occurs in the Presence of ATG-Initiated polyQ ORF and Does not Require Frameshifting.

Most disease-causing CAG.CTG expansions are found in the context of a larger protein expressed in the polyQ frame. To determine if RAN-translation of polyA and polyS proteins occurs from constructs in which translation of polyQ protein is initiated with an upstream ATG and V5-tag, we monitored expression at the C-terminus in all three reading frames with epitope tags (FIG. 13A). Protein blots of transfected HEK293 cells show expression of a ˜40 kDa polyQ protein detected by the V5 and 1C2 antibodies (FIG. 13A). Consistent with our previous results, both polyA (HA-positive) and polyS (Flag-positive) proteins are also expressed in the + and −ATG constructs (FIG. 13A). Additionally, the absence of the V5-tag on the polyQ protein expressed from the (−)ATG V5 construct demonstrates that the majority of non-ATG translation in the polyQ frame starts downstream of the V5 tag (FIG. 13A) and close to or within the repeat tract. The apparent lower molecular weight of the longer protein expressed with the 5′ V5 tag from the +ATG construct (FIG. 13A) is consistent with other observations we have made in which pure polyQ proteins migrate at a higher than expected molecular weights compared to expansion proteins with additional sequence or sequence interruptions.

Although the majority of the 5′V5 tag migrates at the same position as the 40 kDa polyQ protein detected with the 1C2 antibody, immunoprecipitation using antibodies to the 3′His(Q), HA(A) and Flag(S) epitopes followed by immunoblot using the antibodies directed against the 5′ V5 tag show that a relatively small fraction of the total polyA protein has undergone frame shifting from the ATG initiated V5-polyQ frame to the polyA frame (FIG. 19). Although a small amount of frameshifting is detected, these data, and data throughout the rest of the manuscript, show that neither an in-frame ATG initiation codon nor frameshifting are required for translation of polyA, polyQ and polyS proteins.

Non-ATG Translation of CAG Repeat Alone and with Upstream Sequence of HD, HDL2, SCA3 & DM1 Loci.

To investigate the potential relevance of RAN-translation in other expansion disorders, a set of constructs was generated by replacing the upstream ATXN8 sequence with 20 bp of sequence upstream of the CAG from the predicted Huntingtin (HD), Huntingtin-like 2 (HDL2) antisense, spinocerebellar ataxia type 3 (SCA3) or myotonic dystrophy type 1 (DM1) antisense transcripts (FIG. 13B). Each construct has a 6×-STOP cassette and 3′ epitope tags in each frame. RT-PCR shows that each of these constructs express unspliced transcripts with the only ATG-initiated ORFs in the glutamine and serine frames for the A8(*KMQ_{EXP}) and DM1 constructs, respectively (FIG. 13B, shaded). Consistent with the results above, these constructs show robust polyQ and polyA and variable polyS expression with the highest levels of non-ATG polyS translation in the A8(*KKQ_{EXP}) and HDL2 constructs (FIG. 13B). Similarly, RAN translation of polyQ protein also occurs after in vitro transcription of non-ATG containing sequences for the ATT(CAG_{EXP}), HD and HDL2 constructs followed by RNA transfections (FIG. 20) and after lenti-viral transduction of HEK293 cells and mouse brain in which the transgenes (FIG. 21A) integrate into the genome (FIG. 21B and FIG. 21C).

Taken together, these data demonstrate that CAG repeat expansions located within a variety of sequence contexts and under a variety of conditions can express homopolymeric proteins in cells and intact brain in the absence of an ATG start codon.

### Translation of Homopolymeric Expansion Proteins in Reticulocyte Lysates but not HEK293 Cells is Dramatically Affected by Upstream Sequences.

We used a rabbit reticulocyte lysate (RRL) system to test if non-ATG translation also occurs in a cell-free system. As expected, the A8(*KMQ_{EXP}) and DM1 constructs, which have an ATG start codon in the polyQ or polyS frames (FIG. 12A), robustly express the polyQ and polyS proteins in this in vitro system. In contrast to the widespread RAN-translation seen in transfected HEK293 cells (FIG. 13B), non-ATG translation in RRLs is limited to previously described alternative initiation codons differing from the canonical ATG by one nucleotide (ATT and ATC). In RRLs only the HDL2 construct produced the polyQ protein in the absence of an ATG, none of the constructs generated detectable polyA protein, and the highest levels of non-ATG polyS protein were generated from HD and SCA3 constructs. In contrast to RAN-translation in transfected cells, non-ATG translation in cell-free RRLs is substantially affected by mutating previously reported alternative initiation codons (ATT and ATC) (FIG. 12B-D). Additionally, polyQ proteins expressed from non-ATG constructs in the RRL system (FIG. 22) incorporate methionine in the absence of an ATG codon.

### RAN Translation Increases Cell Death in N2a Cells.

To determine if RAN-translation occurs at sufficient levels in cell culture to cause toxicity we transfected murine neuroblastoma N2a cells with CAA- and CAG-expansion constructs with or without an ATG initiation codon and a GFP co-transfection marker. After 48 hours, cells were stained with 7-aminoactinomycin D (7-AAD) and sorted by flow cytometry. FIG. 13C (left) shows the percentage of transfected cells that have undergone cell death and a representative blot showing the relative levels of polyQ, polyA and polyS proteins expressed from each construct in N2a cells. Cells transfected with the ATG(CAA₉₀)-3T constructs expressing only the polyQ protein show no increase in cell death compared to cells transfected with the negative ATT(CAA₉₀)-3T control. In contrast, cells transfected with either the ATT(CAG₁₀₅)-3T or ATG(CAG₁₀₅)-3T show significant increases in cell death compared to the ATT(CAA₉₀)-3T control. These results, demonstrate that RAN translation can be toxic to cells [ATT(CAG₁₀₅)-3T] and additionally suggest that the expression of a mixture of all three proteins [ATG(CAG₁₀₅)-3T] is generally more harmful to cells than the expression of only a single protein [ATG(CAA₉₀)-3T].

### In Vivo Evidence for RAN-Translation in SCA8 and DM1.

To determine if novel homopolymeric proteins predicted by RAN-translation are expressed in vivo, we developed polyclonal antibodies against two putative proteins at the SCA8 and DM1 loci.

First, we developed a polyclonal-rabbit antibody against a unique seven amino-acid stretch (SEQ ID NO:2) located at the C-terminal end of the predicted putative ATXN8-GCA-encoded polyA (SCA8_{GCA-Ala}) protein (FIG. 5A). Protein blot analysis and immunofluorescence staining of transfected cells expressing the SCA8_{GCA-Ala} protein with the predicted endogenous C-terminal sequence demonstrate that the locus specific α-SCA8_{GCA-Ala} polyclonal antibody is able to detect this recombinant SCA8 polyA expansion protein (FIG. 5B). To investigate whether RAN-translation across the ATXN8 CAG expansion transcript occurs in the polyA frame in vivo, we performed immunohistochemistry experiments on an established large insert SCA8 BAC transgenic mouse model previously shown to express SCA8 CAG expansion transcripts and the SCA8 polyQ-expansion protein. Immunohistochemistry experiments using the α-SCA8_{GCA-Ala} antibody consistently show immunoreactivity localized to Purkinje cell soma and dendrites throughout the cerebellum in SCA8 BAC-Exp animals. In contrast, control animals were devoid of any localized immunoreactivity (FIG. 5C, middle and upper panels).

Immunofluorescence staining with the α-SCA8_{GCA-Ala} show that the SCA8_{GCA-Ala} protein is expressed in both Purkinje cell soma and dendrites as well as the granule cell layer (FIG. 5C, lower panels). Additionally, the SCA8_{GCA-Ala} protein was also detected in human Purkinje cells in SCA8 autopsy but not control tissue (FIG. 5D).

In a second set of experiments, polyclonal antibody was generated against a unique 15 amino-acid stretch (SEQ ID NO:5) located at the C-terminal end of the putative DM1-CAG-encoded polyQ (DM1_{CAG-Gln}) protein (FIG. 6A). Protein blots and immunofluorescence staining of transfected HEK293 cells with constructs expressing the DM1_{CAG-Gln} protein with the predicted endogenous C-terminal sequence demonstrate that this antibody can detect a recombinant version of the predicted protein (FIG. 6B) in transfected cells.

Immunofluorescence experiments were performed on mice from an established large insert (45 kb) DM1 mouse model containing CAG.CTG expansions of 55, 328 or >1000 repeats (DM55, DM300, DMSXL) or a normal allele of 20 CTGs (DM20). These mice express DMPK sense transcripts in the CUG direction that accumulate as CUG-containing ribonuclear inclusions (FIG. 23). Additionally, these animals express antisense transcripts in various tissues including transcripts longer than those previously reported which span the repeat in the CAG direction in heart and skeletal muscle (FIG. 24). Similar to the cell culture results, the α-DM1_{CAG-Gln} antibody recognizes nuclear aggregates in cardiac myocytes in DM55, DM300 and DMSXL mice, but not DM20 or non-transgenic controls, examples shown in FIG. 6C with cardiac histology shown in FIG. 10.

When examining the cardiac tissue we noticed additional staining in leukocytes within coagulated blood in the chambers of the heart in the DM55, DM300 and DMSXL expansion mice but not wildtype or DM20 controls, example shown in FIG. 6D. The 1C2 antibody does not adequately detect polyQ inclusions in frozen samples using available methods. Therefore, to independently support that the α-DM1_{CAG-Gln} antibody is detecting the putative DM1_{CAG-Gln} protein is expressed in vivo across expanded CTG repeat tracts, we performed 1C2 immunostaining using paraffin-embedded tissue. 1C2 staining is found in leukocytes in cardiac tissue from mice containing a CTG expansion of 55 repeats but not control mice with 20 CTG repeats (FIG. 6E). Additionally, we double labeled frozen cardiac tissue for the putative glutamine expansion protein and for caspase-8, a protein previously reported to co-localize with other polyQ-expansion proteins in polyQ induced apoptotic cells. Confocal layers through a leukocyte nucleus in the cardiac tissue show α-caspase-8 staining colocalizes with α-DM1_{-CAG-Gln} staining throughout the nucleus (FIG. 6F).

We detected infrequent but reproducible α-DM1_{CAG-Gln} staining in frozen human skeletal muscle from one DM1 autopsy case, but not control tissue (FIG. 27) and show similar co-expression of the DM1-polyQ protein with caspase-8 (FIG. 27B). Additionally, DM1-polyQ inclusions are consistently found at low frequency in myoblasts derived from a patient with (50-70 CTG.CAG repeats) (FIG. 27C). In contrast, the DM1_{CAG-Gln} protein is relatively robustly expressed in patient leukocytes (FIG. 6G). Western analysis of blood from a patient with 85 CTG.CAG repeats using both the αDM1_{CAG-Gln} and 1C2 antibodies shows independent evidence that a DM1 specific polyQ expansion protein is expressed in peripheral blood (FIG. 6H).

### Examples

### cDNA Constructs

A8(*KMQ_{EXP}) was generated by subcloning SCA8 cDNA into pcDNA3.1 vector in the CAG direction. An SCA8 loci containing the CAG repeat expansion was amplified by PCR from the BAC transgene construct BAC-Exp (M. L. Moseley et al., Nat Genet 38, 758 (2006)) using the 5′ primer (5′-CGAACCAAGCTTATCCCAATTCCTTGGCTAGACCC-3′, SEQ ID NO:98) containing an added HindIII restriction site and the 3′ primer (5′-ACCTGCTCTAGATAAATTCTTAAGTAAGAGATAAGC-3′, SEQ ID NO:99) containing an added XbaI restriction site. The HindIII/XbaI PCR product was cloned into the pcDNA3.1/myc-His A vector (Invitrogen Carlsbad, Calif.) in the CAG orientation and placed under the control of the CMV promoter. The ATG start codon in the polyQ frame was mutated into AAG to remove the existing ORF and generate the A8(*KKQ_{EXP}) construct.

To generate the A8(*KMQ_{EXP})-3TF1 and A8(*KKQ_{EXP})-3Tf1, A8(*KKQ_{EXP})-3Tf2, and A8(*KKQ_{EXP})-3Tf3 constructs, the HindIII/XbaI fragment was subcloned into pcDNA3.1/6Stops-3T vector. Stop codons between the 3′ end of the repeat and the tags were subsequently removed. In the resulting constructs, 6 stop codons (two for each frame) were placed prior to the 5′ end of the fragment and each of three reading frames (polyQ, polyA, and polyS) was tagged with myc-His, HA, and Flag epitopes, respectively.

The AATT(CAG_{EXP})-3T construct was made by inserting the PCR fragment containing a pure CAG repeat into the pcDNA3.1/6Stops-3T vector. This construct contains very limited sequence (5′-TAGAATT-CAG-3′, SEQ ED NO:100) between the stop codon cassette and the CAG repeat tract. To remove the sequence between the last 5′ stop codon and the CAG repeat, the AATT(CAG_{EXP})-3T construct was digested with EcoRI, treated with mung bean nuclease, and ligated generating the TAG(CAG_{EXP})-3T construct, in which the last stop codon (TAG) is placed immediately upstream of CAG repeats, eliminating the existence of upstream alternative translation initiation.

To generate the TAAG(CAG_{EXP})-3T construct, PCR was carried out using the 5′ primer (5′-AGTTAAGCTAGCTTAGCTAGGTAACTAAGTAACTAGAATTAA-3′, SEQ ID NO:101) and the 3′ primer (5′-TAGAAGGCACAGTCGAGGCTGATCAGCGGGTTT-3′, SEQ ID NO:102). The PCR product was subcloned into the pcDNA3.1/6Stops-3T vector.

To generate the TAGAG(CAG_{EXP})-3T construct, PCR was carried out using the 5′ primer (5′-AGTTAAGCTAGCTTAGCTAGGTAACTAAGTAACTAGAATAGAGCA-3′, SEQ ID NO:103) and the 3′ primer (5′-TAGAAGGCACAGTCGAGGCTGATCAGCGGGTTT-3′, SEQ ID NO:104). The resulting product was subcloned into the pcDNA3.1/6Stops-3T vector.

The HD-3T, HDL2-3T, SCA3-3T, and DM1-3T constructs were made by inserting the duplex primers containing 20 nt 5′ of the CAG repeats from HD, HDL2, SCA3, and DM1 into the EcoRI site of the ATT(CAG_{EXP})-3T construct. The extra nucleotides between the 5′ flanking sequence (HD, HDL2, SCA3, and DM1) and CAG repeats were removed by digesting with EcoRI and another restriction site on the duplex primers, followed by treatment with mung bean nuclease and DNA ligase.

The NheI/PmeI fragments of A8(*KMQEXP)-3TF1, HD-3T, HDL2-3T, SCA3-3T, and DM1-3T containing 6 stop codons, expanded CAG repeats, and three tags were subcloned into the lentiviral vector, CSII.

The ATG-V5(CAG₁₀₅)-3T construct was created by inserting an oligo (5′-GAATTATGGGTAAGCCTATCCCTAACCCTCTCCTCGGTCTCGAT TCTACGGGA-3′ (SEQ ID NO:105) and 5′-AATTCCCGTAGAATCGAGACCGAGGAGAGGGTTAGGGATAGGCTTACCCAT-3′ (SEQ ID NO:106) containing a V5 tag at the 5′ end of the ATT(CAG_{EXP})-3T construct. The QUICKCHANGE II XL Site-Directed Mutagenesis Kit (Stratagene, Cedar Creek, Tex.) was used to change the ATG in front of the V5 tag to an ATC in order to generate the ATC-V5-(CAG₁₀₅)-3T construct which contains no open reading frames.

To generate the CAA_{EXP} constructs, a CAA repeat was amplified by PCR using the ACA₁₃ and TTG₁₅ primers. PCR products varied in size. A gel slice containing 200-550 bp fragments (67-183 repeats) was purified and the resulting fragments were cloned into the pSC-A-amp/kan vector using STRATACLONE PCR Cloning Kit (Stratagene, Cedar Creek, Tex.). Clones were sequenced and desirable CAA repeats were excised and subcloned into pcDNA3.1/6Stops-3T. The resulting constructs were sequenced and CAA₁₂₅(−ATG), CAA₉₀(−ATG), and CAA₃₈(−ATG) constructs were obtained. Modified versions of these constructs containing an ATG in the polyQ frame [CAA₁₂₅(+ATG), CAA₉₀(+ATG), and CAA₃₈(+ATG)] were created using site directed mutagenesis (Stratagene, Cedar Creek, Tex.).

To generate CTG_{EXP}(Cys-myc/His), CTG_{EXP}(Ala-myc/His), and CTG_{EXP}(Leu-myc/His) constructs, a fragment of expanded CTG repeats was subcloned into pcDNA3.1/myc-His (A, B, and C respectively) and each of the three reading frames were C-terminally tagged. In the three resulting constructs, there is no ORF in each of three frames and polyC, polyA, and polyL are individually tagged in frame with a myc-His tag. Three prime flanking sequence of DM1 in the CAG direction was amplified by PCR using 5′ primer (5′-CTCGAGGCTACAAGGACCCTTCGAG-3′, SEQ ID NO:107) and 3′ primer (5′-CCTGAACCCTAGAACTGTCTTCGACT-3′, SEQ ID NO:108) and cloned into a PCR cloning vector, pCR4-TOPO (Invitrogen).

The XhoI/PmeI fragment of pCR4-DM1-3′ was subcloned downstream of CAG repeats of ATT(CAG_{EXP})-3T to generate the CAG-DM1-3′ construct containing expanded CAG repeats and 3′ flanking sequence of DM1.

The integrity of all constructs was confirmed by sequencing.

PCR mediated mutagenesis was used to create several constructs in which the ATT or ATC alternative start codons were altered to ACT and ACC respectively. All constructs were created using the BGH3-1 3′ primer (5′-TAGAAGGCACAGTCGAGGCTGATCAG CGGGTTT-3′, SEQ ID NO:109) and a unique 5′ primer. The ACT(CAG₁₀₅)-3T Primer (5′-AGTTAAGCTTAGCTAGGTAACTAAGTAACTAGAACTCAGCA-3′, SEQ ID NO:110) was used to generate the ACT(CAG_{EXP})-3T construct from ATT(CAG_{EXP})-3T template. The HDL2-3T:[ATT,ATC] construct was used as template to generate the HDL2-3T:[ATT,ACC], HDL2-3T:[ACT,ATC], and HDL2-3T:[ACT,ACC] constructs from the HDL2:[ATT,ACC] 5-1 (5′-AGTTAAGCTTAGCTAGGTAACTAAGTAACTAGAATTTCCTGCACAGAAAC CACCTT-3′, SEQ ID NO:111), HDL2:[ACT,ATC] 5-1 (5′-AGTTAAGCTTAGCTAGGTAACTAAGTA ACTAGAACTTCCT-3′, SEQ ID NO:112), and HDL2:[ACT,ACC] 5-1 (5′-AGTTAAGCTTAGCTAGGTAACTAAGTAACTAGAACTTCCTGCACAGAAACC ACCTT-3′, SEQ ID NO:113) primers respectively. Likewise, the SCA3:[ACT] construct was generated from SCA3 template and the SCA3:[ACT] 5-1 (5′-AGTTAAGCTTAGCTAGGTAACTAAGTAACTAGAAC TAACA-3′, SEQ ID NO:114) primer. The HD: 5-1 primer (5′-AGTTAAGCTTAGCTAGGTAACTAAGTAACTAGAACTTCGA-3′, SEQ ID NO:115) was used along with HD-3T:[ATT] template to generate the HD-3T:[ACT] construct.

All PCR reactions to generate the above constructs were performed with Pfx polymerase (Invitrogen, Carlsbad, Calif.) to mitigate PCR-induced mutations. PCR conditions: Initial denaturation was performed at 94° C. for two minutes followed by 35 cycles of 94° C. for one minute, 55° C. for one minute, and 72° C. for one minute. Final extension was done at 72° C. for 10 minutes. PCR Products were subjected to a phenol extraction/ethanol precipitation and resuspended in 50 μl dH2O. Derivatives of the HDL2:[ATT,ACT] construct were digested with HindIII and PmeI, gel purified and cloned into a phosphatased pcDNA3.1 vector containing the 6× stop cassette. The integrity of all constructs was confirmed by sequencing.

### Production of Polyclonal Antibodies

The polyclonal antibodies were generated by New England Peptide (Gardner, Mass.). The α-SCA8_{GCA-Ala} antisera were raised against a synthetic peptide corresponding to the C-terminus of a predicted polyA frame of SCA8 in the CAG direction (VKPGFLT, SEQ ID NO:2). The α-DM1_{CAG-Gln} antisera were raised against a synthetic peptide corresponding to the C-terminus of a predicted glutamine frame of DM1 in the CAG direction (SPAARGRARITGLEL, SEQ ID NO:5).

### Cell Culture, Transfection, and Immunofluorescence

HEK293 cells were cultured in DMEM medium supplemented with 10% fetal bovine serum and incubated at 37° C. in a humid atmosphere containing 5% CO₂. DNA transfections were performed using Lipofectamine 2000 Reagent (Invitrogen) according to the manufacturer's instructions.

DM1 patient myoblasts with 50-70 CTG repeats, along with a normal control, were cultured in SGM (Promocell, Heidelberg, Germany) with Glutamax, Gentamicin 50 u/ml, decomplemented fetal calf serum and the provided supplemental mix. Cells were grown to approximately 70% confluence on collagen coated coverslips in 6-well tissue-culture plates.

### RNA Transfections

Plasmid DNA was linearized using PvuII. Transcription, capping, and polyadenylation was performed using 1 μg of DNA with the mScript mRNA Production System (Epicentre, Wis.). Transfections were performed in 6-well plates using 3 μg of mRNA and 10 μl Lipofectamine 2000 (Invitrogen) per well. Cell lysates were collected 18-24 hours post transfection and immunoblots were performed as described.

### Immunofluorescence

The subcellular distribution of homopolymer proteins was assessed in transfected HEK293 cells by immunofluorescence. Cells were cultured on coverslips in six-well tissue culture plates and transfected with plasmids the next day. Forty-eight hours post-transfection, cells were fixed in 4% paraformaldehyde in PBS for 30 minutes and permeabilized in 0.5% Triton X-100 in PBS for 10 minutes. The coverslips were blocked in 1% normal goat serum in PBS for 30 min. After blocking, the cells were incubated for 1 hour at 37° C. in blocking solution containing primary antibodies rabbit anti-His (1:100), rat anti-HA (1:100), and mouse anti-Flag (1:200). The coverslips were washed three times in PBS and incubated for 1 hour at 37° C. in blocking solution containing secondary antibodies. Goat anti-rabbit conjugated to Cy3 (Jackson ImmunoResearch West Grove, Pa.), goat anti-rat conjugated to Cys5 ((Jackson ImmunoResearch), and goat anti-mouse conjugated to ALEXA FLUOR 488 (Invitrogen) were used at a dilution of 1:200.

DM1 patient myoblasts grown on coverslips were fixed in 4% paraformaldehyde for 30 minutes and blocked with 5% normal goat serum for one hour. Next, the cells were incubated with α-DM1_{CAG-Gln}) (1:5,000) at 4° C. overnight. Cells were then washed and incubated with Goat anti-rabbit conjugated to Cy3 (Jackson ImmunoResearch) for one hour at room temperature, in darkness. Slides were washed 3×5 minutes in 1×PBS, mounted with Vectashield Hard set mounting medium with DAPI (Vector Laboratories, Inc. CA) and coverslipped.

For mouse and human tissues, 9 μm cryosections were fixed in 4% paraformaldehyde for 15 minutes. Heat induced epitope retrieval (HIER) was employed by steaming sections in citrate buffer, pH 6.0, at 90° C. for 20 minutes. HIER was used in all IF tissue experiments except for SCA8_{GCA-Ala} mouse and human experiments in which antigen retrieval was omitted altogether. A non-serum block (Biocare Medical LLC, Concord, Calif.) was applied to all tissues, except the SCA8 mouse tissue in which 10% normal goat serum (NGS) in a 0.3% Triton-X-100 was used to block non-specific immunoglobulin binding, and allowed to incubate at room temperature for one hour. The primary antibody/antibodies (if double or triple labeled) of interest were either diluted in a 1:5 solution of the non-serum block or a 5% NGS in PBS solution containing 0.3% Triton X-100 and incubated at 4° C. overnight. Tissues were then incubated for one hour in a 1:2,000 dilution of IgG-TRIC, in the dark, at room temperature. If needed, a Sudan-black autofluorescence block was applied to the tissue for 1 hr at room temperature in the dark. Staining was observed and pictures were taken on an FLUOVIEW 1000 IX2 inverted confocal microscope (Olympus America Inc., Center Valley, Pa.). All mutant and control images were adjusted in unison, to the same specifications, and in a linear fashion, for intensity and contrast when deemed necessary.

Labeling PolyQ Protein with [³⁵S]-Methionine

A T7-coupled transcription and translation kit (Promega, Madison, Wis.) was used with these templates to generate polyQ proteins labeled with [³⁵S]-methionine (MP Biomedicals LLC, Solon, Ohio). Labeled proteins were run out in parallel on two separate gels. One gel was subsequently dried and used to generate an autoradiograph while the other was used for a western blot. Western blot was probed with the 1C2 antibody.

### Immunofluorescence Staining of Mouse and Human Tissues

Nine micrometer cryosections were fixed in 4% paraformaldehyde for 15 min. Heat induced epitope retrieval (HIER) was employed by steaming sections in citrate buffer, pH 6.0, at 90° C. for 20 min. HIER was used in all IF tissue experiments except for SCA8_{GCA-Ala} mouse and human experiments in which antigen retrieval was omitted altogether. A non-serum block (#BS966, Biocare Medical LLC, Concord, Calif.) was applied to all tissues, except the SCA8 mouse tissue in which 10% normal goat serum (NGS) in a 0.3% Triton X-100 was used to block non-specific immunoglobulin binding, and allowed to incubate at room temperature for one hour. The primary antibody/antibodies (if double or triple labeled) of interest were either diluted in a 1:5 solution of the non-serum block or a 5% NGS in PBS solution containing 0.3% Triton X-100 and incubated at 4° C. overnight. Tissues were then incubated for 1 hour in a 1:2,000 dilution of IgG-TRIC, in the dark, at room temperature. If needed, a Sudan-black autofluorescence block was applied to the tissue for 1 hr at room temperature in the dark (33). Staining was observed and pictures taken on an FLUOVIEW 1000 IX2 (Olympus America Inc., Center Valley, Pa.) inverted confocal microscope.

### Immunohistochemistry

DM mutant and control mice were perfused in 10% formalin and tissue harvested and embedded in paraffin. 5 μm sections were deparaffinized in xylene and rehydrated through graded alcohol, incubated with 90% formic acid for 5′ and washed with distilled H₂O for 30 min. HIER was performed by steaming sections in citrate buffer, pH 6.0, at 90° C. for 20 min. To block non-specific avidin-D/biotin binding, the Avidin-D/Biotin block was used as described (#SP-2100 Vector Labs, Burlingame, Calif.). To block non-specific immunoglobulin binding, a non-serum block (#BS966, Biocare Medical LLC, Concord, Calif.) was applied for 30 minutes. Primary 1C2 antibody was applied at a dilution of 1/12,000 in non-serum block (#BS966, Biocare Medical LLC, Concord Calif.) and incubated overnight at 4° C. Biotinylated secondary α-mouse IgG purified in goat (#BA-9200, Vector Labs, Burlingame, Calif.) was applied at a dilution of 1:200 for 30′ at RT. ABC reagent (PK-7100, Vector Lab, Burlingame, Calif.) was used for detection with CHROMAGEN SG (#SK-4700, Vector Lab, Bulingame, Calif.) for 10 minutes and counterstained with nuclear fast red.

Leukocyte cell pellets were isolated from peripheral blood of DM1 and control patients. The cell pellets were fixed in 10% neutral buffered formalin for 30 minutes, washed, encapsulated in HistoGel™ (Richard-Allen, Kalamazoo, Mich.), and placed in 70% ETOH. The pellets then underwent a short, two hour cycle in the tissue processor and were embedded in paraffin blocks. 5 μm sections were cut, deparaffinised, and hydrated to water. HIER was employed with steam and Reveal Decloaker (Biocare Medical LLC, Concord, Calif.). A non-serum block (Biocare Medical LLC, Concord, Calif.) was applied for 30 minutes to prevent non-specific immunoglobulin binding. The nonserum block 1:10 in PBS was used to dilute the α-DM1_{CAG-Gln}) Ab to a concentration of 1:10,000. Slides were incubated overnight at 4° C., and washed 3×5 minutes in PBS. The Secondary antibody, DyLight™ 488-conjugated AffiniPure Goat Anti Rabbit, (Jackson Immunoresearch) was applied and incubated for two hours in the dark, at room temperature, and at a concentration of 1:1,000. Slides were washed 3×5 minutes in PBS, mounted with Vectashield Hard Set Mounting Medium with DAPI (Vector Labs, Burlingame, Calif.) and coverslipped. Staining was observed and pictures taken on an Olympus FluoView 1000 IX2 inverted confocal microscope. For consistency in FIG. 6, Olympus Fluoview software was used to reassign the 488, (green) captured signal to red.

### Cell Death Analysis

For flow cytometric Annexin V and propidium iodide analysis, floating cells were collected and combined with trypsinized, adherent cells in cold PBS. After washing, cells were resuspended in Annexin binding buffer (BD Biosciences, San Jose, Calif.), vortexed, and stained with Annexin V-APC (BD Biosciences, San Jose, Calif.) and propidium iodide (BD Biosciences, San Jose, Calif.) according to BD Pharmingen instructions. Cells were placed on ice and immediately sorted on a BD FACScalibur flow cytometer. Thirty-thousand total events were collected.

Three independent experiments were performed and data combined and normalized to the ATT(CAA₉₀) average. Statistics were performed using a one-way ANOVA and p values calculated with a one-tailed t-test.

Labeling and Immunoprecipitation of polyQ, polyA and polyS Proteins with [³H]-Amino Acids

HEK293 cells were cultured in DMEM medium supplemented with 10% fetal bovine serum and transfected with CAG expansion construct. Twenty-four hours post-transfection, the DMEM-based medium was replaced with the glutamine-, alanine-, and serine-free MEM medium (Invitrogen) supplemented with 10% fetal bovine serum. Then [³H]-glutamine, [³H]-alanine, or [³H]-serine was added into the respective wells at 25 μCi/ml and the cells were incubated for 16 hours at 37° C. Cells in culture plates are rinsed with PBS and lysed in RIPA buffer (150 mM NaCl, 1% sodium deoxycholate, 1% Triton X-100, 50 mM Tris-HCl pH 7.5, 1× protease inhibitors (Roche, Madison, Wis.) for 45 minutes on ice. The cell lysates were centrifuged at 16,000×g for 15 minutes at 4° C. and the supernatant was collected. To immunoprecipitate ³H-labeled protein, 500 μg of tissue lysate was incubated with the desired antibody at 4° C. for two hours and then with protein G-Sepharose at 4° C. overnight. Protein G-Sepharose was washed three times with RIPA buffer. Bound proteins were eluted from the beads with 1×SDS sample buffer, incubated at 90° C. for 10 minutes, and analyzed by protein gel electrophoresis.

### Immunoprecipitation

The protein concentration of tissue lysates was determined using the protein assay dye reagent (Bio-Rad Laboratories, Hercules, Calif.). To immunoprecipitate polyQ protein, 500 μg of tissue lysate was incubated with rabbit polyclonal anti-His antibody at 4° C. for two hours and then with protein G-Sepharose at 4° C. overnight. Protein G-Sepharose was washed three times with RIPA buffer. Bound proteins were eluted from the beads with 1×SDS sample buffer, boiled for 10 min, and analyzed by immunoblotting.

### Immunoblotting

Cells in each well of a six-well tissue culture plate were rinsed with PBS and lysed in 300 μl RIPA buffer (150 mM NaCl, 1% sodium deoxycholate, 1% Triton X-100, 50 mM Tris-HCl pH 7.5, 1× protease inhibitors) for 45 min on ice. DNA was sheared by passage through a 21-gauge needle. The cell lysates were centrifuged at 16,000×g for 15 min at 4° C. and the supernatant was collected. The protein concentration of the cell lysate was determined using the protein assay dye reagent (Bio-Rad Laboratories, Inc., Hercules, Calif.). Twenty micrograms of protein were separated in a 4-12% or 10% NuPAGE Bis-Tris gel (Invitrogen) and transferred to nitrocellulose membrane (Amersham, Piscataway, N.J.). The membrane was blocked in 5% dry milk in PBS containing 0.05% Tween 20 and probed with the anti-His antibody (1:500) or 1C2 antibody (1:1,000) in blocking solution. After incubating the membrane with anti-rabbit or anti-mouse HRP conjugated secondary antibody (Amersham), bands were visualized by ECL plus Western Blotting Detection System (Amersham).

### Mass Spectrometry

To immunoprecipitate polyA protein for mass spectrometry, transfected HEK293 cell lysate from five 150-mm dishes was incubated with mouse monoclonal antibody against C-terminal tag at 4° C. for two hours and then with protein G-Sepharose at 4° C. for overnight. Protein G-Sepharose was washed three times with RIPA buffer. Bound proteins were eluted from the beads with 8M urea.

Samples were separated by parallel SDS-PAGE 4-15% Criterion Tris-HCl gels (Bio-Rad Laboratories, Hercules, Calif.), one for mass spectrometry preparation and the other for immunoblotting. Protein bands of interest were excised manually after visualizing with Imperial™ Protein Stain (Thermo Scientific). Specified bands were cut out and subjected to in-gel trypsin digestion using standard methods and extracted peptides were further cleaned up using “stage” tips.

Mass analysis was performed using an LTQ-Orbitrap XL mass spectrometer (ThermoScientific). Peptides derived from in-gel digestion were separated by reversed phase chromatography with nanoHPLC. The gradient was 2-40% acetonitrile in H₂O containing 0.1% formic acid over 60 minutes. Full MS scans were generated in the orbital trap at 60,000 resolution for 400 m/z. MS/MS scans were performed in a data dependent manner using an inclusion list based on predicted tryptic peptides in the LTQ ion trap using CID. Data were searched with SEQUEST v.27 with semi-trypsin specificity, Cys carbamidomethylation as a fixed modification, and N-terminal acetylation and Met oxidation as variable modifications. The search was performed against the combined database consisting of the NCBI human database V200906 and its reversed complement and an additional list of all possible proteins that could be initiated anywhere in the polyalanine frame of the Interrupt(CAG)exp-3T construct with or without an N-terminal methionine, which totaled >76,000 entries. Identified proteins were organized using SCAFFOLD (Proteome Software, Inc., Protland, Oreg.) and peptide probabilities were calculated within this program using Peptide Prophet. The identification output was filtered using a precursor mass tolerance at 7 ppm.

### In Vitro Translation

In vitro translation was performed using coupled reticulocyte lysate systems (Promega, Madison, Wis.). Coupled transcription/translation reactions (50 μl) contained 50% lysate, 1 μl of T7 RNA polymerase, 20 μM amino acid mixtures, 40 μl ribonuclease inhibitor and 1 μg of plasmid DNA; incubation was at 30° C. for 90 min. Ten percent of each reaction was analyzed by western blotting.

### Production and Purification of Lentiviral Vectors and Transduction of HEK293 Cells

HEK293 cells were plated on 150-mm tissue culture dishes and transfected the following day when cells were 80-90% confluent. Thirty micrograms of the transducing vector, 20 μg of the packaging vector ΔNRF, and 10 μg of the VSV envelope pMD.D were co-transfected by calcium phosphate-mediated transfection. The medium was changed the next day, and conditioned media were collected 48 and 72 hours after transfection. Conditional medium was then cleared by filtering though a 0.45-μm filter. The viral particles were concentrated by ultracentrifugation at 50,000×g for 2 hours. The pellet was resuspended in 20 μl of 1×HBSS and stored at −70° C. HEK293 cells were seeded into each well of a six-well plate and transduced the next day. Transduced cells were analyzed by western blotting after 5 days.

Injection of Mouse Brain with Lentiviral Vectors

Six-week old FVB mice were anesthetized by intramuscular injection using a combination of ketamine and xylazine. Two microliters of lentiviral vectors (5 10⁹ TU/ml) were injected into mouse striatum and cerebellum respectively. The mouse was mounted in a stereotactic frame and its head shaved. A midline sagittal incision was made and the cranium was exposed. For each injection site, a burr hole was drilled and a Hamilton syringe was inserted to the depth described below the dura, plus an additional 0.5 mm. After 2 min, the syringe was retracted 0.5 mm, to form a slight pocket in the parenchyma. After a pause of at least 2 min for pressure equalization, the injection was performed manually at an approximate rate of 0.5 μl per minute. Afterwards, the syringe was left in place an additional 3 min, and then withdrawn over a period of 2 min or more. Once injections were complete, the scalp was sutured and the mouse kept under a warming lamp until recovered from the anesthesia, and returned to standard housing. Animal care followed the guidelines set by the Institutional Animal Care and Use Committee at the University of Minnesota.

### Polysome Profiling

Transfected HEK293 cells in 150-mm dishes were treated with cycloheximide (100 μg/ml) for 5 minutes and harvested by trypsinization. The cell pellet was resuspended in 375 μl of low salt buffer (10 mM NaCl, 20 mM tris pH 7.5, 3 mM MgCl₂ 1 mM DTT, 200 U RNAse inhibitor) and allowed to swell for two minutes. 125 μl of lysis buffer (0.2 M sucrose, 1.2% Triton X-100 in LSB) was added and the cells were homogenized using 15 strokes in a Dounce homogenizer using the tight fitting pestle. Lysate was centrifuged at 16,000 g for one minute, and the nuclear pellet was removed. Cytoplasmic extract (1.5 mg measured at A₂₆₀) was layered onto a 5 ml, 0.5-1.5 M sucrose gradient and centrifuged at 200,000 g in a Beckman SW50 rotor for 80 minutes at 4° C. The gradients were fractionated using an ISCO density gradient fractionator monitoring absorbance at 254 nm. Ten fractions were collected from each sample into tubes containing 50 μl of 10% SDS.

### Northern Analysis

The RNA from each fraction of the sucrose gradient was extracted using Tri-reagent (Sigma). For Northern blot analysis, equal volume of the RNA from each fraction was separated on a glyoxal gel, blotted to a nylon membrane, and probed with a [³²P]ATP-labeled oligonucleotide (5′-TAGAAGGCACAGTCGAGGCTGATCAGCGGGTTTAAACTCAAT-3′, SEQ ID NO:116) complementary to the 3′ end of the CAG-containing transcripts. Blots were subsequently probed with a [³²P]dATP-labeled GAPDH cDNA probe.

### RT-PCR

For detection of CAG and CAA expansion transcripts, cells were transfected using Lipofectamine 2000 (Invitrogen) as described above. RNA and protein were harvested using Trizol (Invitrogen). Approximately 45 μg of RNA from each sample was resuspended in 50 μl DEPC dH2O. The RNA sample was treated with an RNase-Free DNase Set (Qiagen, CA) and the RNeasy Plus Mini Kit (Qiagen, Valencia, Calif.) to remove DNA. A Superscript II Reverse Transcriptase System (Invitrogen) and the Myc Tag GSP Primer (5′-CAGATCCTCTTCTGAGATGAGTTTTTGTTC-3′, SEQ ID NO:117) were used to reverse transcribe the RNA and PCR was performed using the 336 F (5′-ACCCAAGCTGGCTAGTTAAGC-3′, SEQ ID NO:118) and 336 R (5′-TGTCGTCGTCGTCCTTGTAA-3′, SEQ ID NO:119) primers at 95° C. for 2 minutes, then 35 cycles of 94° C. for 45 seconds, 59.5° C. for 30 seconds, 72° C. for 45 seconds, and 6 minutes extension at 72° C. Control reactions were performed using the β-actin F (5′-TCGTGCGTGACATTAAGGAG-3′, SEQ ID NO:120) and β-actin R (5′-GATCTTCATTGTGCTGGGTG-3′, SEQ ID NO:121) primers. PCR conditions: 95° C. for 2 minutes, then 35 cycles of 94° C. for 45 seconds, 59.5° C. for 30 seconds, 72° C. for 45 seconds, followed by a 6 minute final extension at 72° C. PCR products were separated on a 1% agarose gel. For detection of CAG expansion transcripts in DM humans and mice, total RNA was extracted from frozen tissues with Trizol (Invitrogen) following incubation with lysis buffer and 0.5 mg/ml proteinase K, as well as precipitation and DNAse treatment. For strand-specific RT-PCR, an lk linker sequence was attached (5′-CGACTGGAGCACGAGGACACTGA-3′, SEQ ID NO:122) to the 5′ end of primers specific for the antisense strand of DMPK:1,5′-CGCCTGCCAGTTCACAACCGCTCCGAGCGT-3′, SEQ ID NO:123; or DMPK:2, 5′-GACCATTTCTTTCTTTCGGCCAGGCTGAGGC-3′ SEQ ID NO:124. Three μg of RNA were reverse transcribed with Superscript III (Invitrogen) at 55° C. PCR against the anti1B, antiN3, and antiA2 regions was carried out using the CTCF1b (5′-GCAGCATTCCCGGCTACAAGGACCCTTC-3′, SEQ ID NO:125), AntiN3 (5′-GAGCAGGGCGTCATGCACAAG-3′, SEQ ID NO:126) and the AntiA2 (5′-TAGGTGGGGACAGACAAT-3′, SEQ ID NO:127) primers, respectively. The linker primer was used in all reactions. The PCR reactions were done using the following conditions: antiB1, 94° C. for 5 minutes then 30 cycles of 94° C. for 30 seconds, 67° C. for 30 seconds and 72° C. for one minute followed by 10 minutes at 72° C.; antiN3, 94° C. for 5 minutes then 30 cycles of 94° C. for 30 seconds, 63° C. for 30 seconds and 72° C. for one minute followed by 10 minutes at 72° C.; antiA2, 94° C. for 5 minutes then 40 cycles of 94° C. for 30 seconds, 57° C. for 30 seconds and 72° C. for one minute followed by 10 minutes at 72° C. Gapdh was amplified using the GFw (5′-AGGTCGGTGTGAACGGATTTG-3′, SEQ ID NO:128) and GRev (5′-TGTAGACCATGTAGTTGAGGTCA-3′, SEQ ID NO:129) primers at 94° C. for 5 minutes then 24 cycles of 94° C. for 30 seconds, 65° C. for 30 seconds and 72° C. for one minute followed by 10 minutes at 72° C.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. All headings are for the convenience of the reader and should not be used to limit the meaning of the text that follows the heading, unless so specified.

Sequence Listing Free Text

SEQ ID NO: 1
LPHTAYLLLKNL
SEQ ID NO: 2
VKPGFLT
SEQ ID NO: 3
RVNLSVEAGSQKRQSE
SEQ ID NO: 4
ATRTLRAPFAGRG
SEQ ID NO: 5
SPAARGRARITGLEL
SEQ ID NO: 6
AVPRALSLPTGPRSRRQF
SEQ ID NO: 7
ITDHFFLSARLR
SEQ ID NO: 8
GSQTISFFRPG
SEQ ID NO: 9
GKLQAWEGSKPGR
SEQ ID NO: 10
LKGEFQHTGGRSL
SEQ ID NO: 11
SDLIKRQDEDRFA
SEQ ID NO: 12
LPACLPACLPACLPAC
SEQ ID NO: 13
QAGRQAGRQAGRQAGR

1. An isolated polypeptide comprising:
at least six contiguous amino acids of a RAN-translated polypeptide comprising:
at least six contiguous amino acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11;
at least six contiguous amino acids of the N-terminal sequence of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96; or
at least six contiguous amino acids of the C-terminal sequence of any one or more of SEQ NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ ID NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97.

2. An isolated polypeptide comprising:
a repeat portion comprising at least five contiguous amino acids; and
a non-repeat portion comprising a:
at least six contiguous amino acids of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11;
at least six contiguous amino acids of an N-terminal sequence of a RAN-translated polypeptide; or
at least six contiguous amino acids of an C-terminal sequence of a RAN-translated polypeptide.

3. The isolated polypeptide of claim 2 wherein the repeat portion comprises at least five contiguous repeated leucine residues and the non-repeat portion comprises at least at least six contiguous amino acids of any one or more of SEQ ID NO:1, SEQ ID NO:8, SEQ ID NO:19, SEQ ID NO:21, SEQ ID NO:36, SEQ ID NO:40, SEQ ID NO:42, SEQ ID NO:47, SEQ ID NO:58, SEQ ID NO:64, SEQ ID NO:69, SEQ ID NO:72, SEQ ID NO:77, SEQ ID NO:83, SEQ ID NO:89, or SEQ ID NO:92.

4. The isolated polypeptide of claim 2 wherein the repeat portion comprises at least five contiguous repeated alanine residues and the non-repeat portion comprises at least six contiguous amino acids of any one or more of SEQ ID NO:2, SEQ ID NO:4, SEQ ID NO:7, SEQ ID NO:14, SEQ ID NO:18, SEQ ID NO:32, SEQ ID NO:35, SEQ ID NO:37, SEQ ID NO:39, SEQ ID NO:41, SEQ ID NO:44, SEQ ID NO:46, SEQ ID NO:51, SEQ ID NO:52, SEQ ID NO:55, SEQ ID NO:57, SEQ ID NO:61, SEQ ID NO:63, SEQ ID NO:65, SEQ ID NO:68, SEQ ID NO:71, SEQ ID NO:74, SEQ ID NO:76, SEQ ID NO:79, SEQ ID NO:81, SEQ ID NO:82, SEQ ID NO:85, SEQ ID NO:88, SEQ ID NO:91, SEQ ID NO:94, or SEQ ID NO:96.

5. The isolated polypeptide of claim 2 wherein the repeat portion comprises at least five contiguous repeated serine residues and the non-repeat portion comprises at least six contiguous amino acids of any one or more of SEQ ID NO:3, SEQ ID NO:6, SEQ ID NO:16, SEQ ID NO:33, SEQ ID NO:38, SEQ ID NO:39, SEQ ID NO:56, SEQ ID NO:66, SEQ ID NO:70, SEQ ID NO:80, SEQ ID NO:86, SEQ ID NO:90, SEQ ID NO:95, or SEQ ID NO:97.

6. The isolated polypeptide of claim 2 wherein the repeat portion comprises at least five contiguous repeated glutamine residues and the non-repeat portion comprises at least six contiguous amino acids of any one or more of SEQ ID NO:5, or SEQ ID NO:37.

7. The isolated polypeptide of claim 2 wherein the repeat portion comprises at least five contiguous repeated cysteine residues and the non-repeat portion comprises at least six contiguous amino acids of any one or more of SEQ ID NO:9, SEQ ID NO:34, SEQ ID NO:40, SEQ ID NO:41, SEQ ID NO:48, SEQ ID NO:59, SEQ ID NO:67, SEQ ID NO:73, SEQ NO:78, SEQ ID NO:84, SEQ NO:87, SEQ ID NO:93, or SEQ ID NO:95.

8. The isolated polypeptide of claim 2 wherein the repeat portion comprises at least six contiguous amino acids of SEQ ID NO:12 and the non-repeat portion comprises at least six contiguous amino acids of any one or more of SEQ ID NO:10, SEQ ID NO:11, SEQ ID NO:26, SEQ ID NO:27, or SEQ ID NO:28.

9. The isolated polypeptide of claim 2 wherein the repeat portion comprises at least six contiguous amino acids of SEQ ID NO:13 and the non-repeat portion comprises at least six contiguous amino acids of SEQ ID NO:31.

10. The isolated polypeptide of claim 2 wherein the non-repeat portion comprises at least one amino acid from an N-terminal sequence or a C-terminal sequence.

11. The isolated polypeptide of claim 2 wherein the N-terminal sequence, if present, comprises of any one or more of SEQ ID NO:14, SEQ ID NO:16, SEQ ID NO:18, SEQ ID NO:19, SEQ ID NO:20, SEQ ID NO:22, SEQ ID NO:24, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:54, SEQ ID NO:55, SEQ ID NO:56, SEQ ID NO:58, SEQ ID NO:59, SEQ ID NO:66, SEQ ID NO:68, SEQ ID NO:69, SEQ ID NO:71, SEQ ID NO:72, SEQ ID NO:73, SEQ ID NO:74, SEQ ID NO:75, SEQ ID NO:76, SEQ ID NO:80, SEQ ID NO:81, SEQ ID NO:87, SEQ ID NO:88, SEQ ID NO:90, SEQ ID NO:91, SEQ ID NO:92, SEQ ID NO:93, SEQ ID NO:94, SEQ ID NO:95, or SEQ ID NO:96; and
the C-terminal sequence, if present, comprises any one or more of SEQ ID NO:14, SEQ ID NO:16, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:29, SEQ ID NO:30, SEQ ID NO:31, SEQ ID NO:32, SEQ ID NO:33, SEQ ID NO:34, SEQ ID NO:36, SEQ ID NO:37, SEQ ID NO:38, SEQ ID NO:39, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:56, SEQ ID NO:57, SEQ ID NO:58, SEQ NO:59, SEQ ID NO:60, SEQ ID NO:61, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, or SEQ ID NO:97.

12. An antibody composition that specifically binds to a polypeptide of claim 1.

13. A method comprising:
receiving a biological sample from a subject;
detecting whether the biological sample comprises a RAN-translated polypeptide associated with a condition characterized at least in part by a nucleotide repeat expansion; and
identifying the subject as at risk for a condition characterized by a repeat expansion if the biological sample includes the RAN-translated polypeptide.

14. A method comprising
receiving a biological sample from a subject being treated for a condition characterized at least in part by a repeat expansion;
measuring the amount of at least one biomarker indicative of a repeat expansion in the biological sample; and
quantifying any change in the amount of biomarker in the sample with respect to a reference value of the amount of biomarker in a sample obtained prior to the subject being treated for the condition.

15. The method of claim 14 further comprising modifying the treatment if the change in the biomarker is less than a standard value indicative of efficacious treatment.

16. A method for analyzing a subject's risk for developing a condition characterized at least in part by a nucleotide repeat expansion, the method comprising:
receiving at least a first biological sample and a second biological sample from a subject, wherein at least one of the following is true:
the first biological sample and the second biological sample were obtained from the subject at different times, or
the first biological sample and the second biological sample were obtained from different tissues;
measuring the amount of at least one biomarker indicative of a repeat expansion in each of the biological samples; and
identifying any difference in the biomarker between the first biological sample and the second biological sample.

17. The method of claim 16 further comprising quantifying any difference in the biomarker between the first biological sample and the second biological sample.

18. The method of claim 13 wherein the condition comprises Type 1 myotonic dystrophy (DM1) or Type 2 myotonic dystrophy (DM2).

19. The method of claim 13 wherein the condition comprises Huntington's Disease (HD) or Huntington's Disease-like 2 (HDL2).

20. The method of claim 13 wherein the condition comprises Fragile X Syndrome (FRAXA).

21. The method of claim 13 wherein the condition comprises Spinal Bulbar Muscular Atrophy (SMBA).

22. The method of claim 13 wherein the condition comprises Dentatorubropallidoluysian Atrophy (DRPLA).

23. The method of claim 13 wherein the condition comprises Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17).

24. The method of claim 13 wherein the condition is at least partially characterized by a repeat expansion at the CTG18.1 locus.

25. The method of claim 16 wherein the first biological sample and the second biological sample were obtained from the subject at different times; and further comprising identifying that the subject as at risk for the condition if the biomarker is present in a greater amount in the biological sample obtained at a later time.

26. The method of claim 13 wherein detecting whether the biological sample comprises a RAN-translated polypeptide associated with a condition characterized at least in part by a nucleotide repeat expansion comprises contacting at least a portion of the biological sample with an antibody that specifically binds to a RAN-translated polypeptide and determining whether the antibody specifically binds to a component of the biological sample.

27. The method of claim 14 wherein measuring the amount of at least one biomarker comprises contacting at least a portion of the biological sample with an antibody that specifically binds to the biomarker and measuring the amount of antibody that specifically binds to a component of the biological sample.

28. A polynucleotide encoding the polypeptide of claim 2.

29. Canceled.

30. An antibody composition that specifically binds to a polypeptide of claim 2.

31. The method of claim 14 wherein the condition comprises Type 1 myotonic dystrophy (DM1) or Type 2 myotonic dystrophy (DM2).

32. The method of claim 14 wherein the condition comprises Huntington's Disease (HD) or Huntington's Disease-like 2 (HDL2).

33. The method of claim 14 wherein the condition comprises Fragile X Syndrome (FRAXA).

34. The method of claim 14 wherein the condition comprises Spinal Bulbar Muscular Atrophy (SMBA).

35. The method of claim 14 wherein the condition comprises Dentatorubropallidoluysian Atrophy (DRPLA).

36. The method of claim 14 wherein the condition comprises Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17).

37. The method of claim 14 wherein the condition is at least partially characterized by a repeat expansion at the CTG18.1 locus.

38. The method of claim 15 wherein the condition comprises Type 1 myotonic dystrophy (DM1) or Type 2 myotonic dystrophy (DM2).

39. The method of claim 15 wherein the condition comprises Huntington's Disease (HD) or Huntington's Disease-like 2 (HDL2).

40. The method of claim 15 wherein the condition comprises Fragile X Syndrome (FRAXA).

41. The method of claim 15 wherein the condition comprises Spinal Bulbar Muscular Atrophy (SMBA).

42. The method of claim 15 wherein the condition comprises Dentatorubropallidoluysian Atrophy (DRPLA).

43. The method of claim 15 wherein the condition comprises Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17).

44. The method of claim 15 wherein the condition is at least partially characterized by a repeat expansion at the CTG18.1 locus.

45. The method of claim 16 wherein the condition comprises Type 1 myotonic dystrophy (DM1) or Type 2 myotonic dystrophy (DM2).

46. The method of claim 16 wherein the condition comprises Huntington's Disease (HD) or Huntington's Disease-like 2 (HDL2).

47. The method of claim 16 wherein the condition comprises Fragile X Syndrome (FRAXA).

48. The method of claim 16 wherein the condition comprises Spinal Bulbar Muscular Atrophy (SMBA).

49. The method of claim 16 wherein the condition comprises Dentatorubropallidoluysian Atrophy (DRPLA).

50. The method of claim 16 wherein the condition comprises Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17).

51. The method of claim 16 wherein the condition is at least partially characterized by a repeat expansion at the CTG18.1 locus.

52. The method of claim 15 wherein measuring the amount of at least one biomarker comprises contacting at least a portion of the biological sample with an antibody that specifically binds to the biomarker and measuring the amount of antibody that specifically binds to a component of the biological sample.

53. The method of claim 16 wherein measuring the amount of at least one biomarker comprises contacting at least a portion of the biological sample with an antibody that specifically binds to the biomarker and measuring the amount of antibody that specifically binds to a component of the biological sample.
